SEQUENCE LISTING (160 sequences)

SEQ ID NO: 1 - DNA, 19 bp, unidentified
  source 1..19: mol_type = other DNA
gcttctggcaaaggatatt

SEQ ID NO: 2 - DNA, 19 bp, unidentified
  source 1..19: mol_type = other DNA
cttcagggctgtggacaac

SEQ ID NO: 3 - DNA, 19 bp, unidentified
  source 1..19: mol_type = other DNA
ccacacagctggacgacta

SEQ ID NO: 4 - DNA, 19 bp, unidentified
  source 1..19: mol_type = other DNA
tccgtggctacttcaagca

SEQ ID NO: 5 - DNA, 19 bp, unidentified
  source 1..19: mol_type = other DNA
aaggctatgcttctggcaa

SEQ ID NO: 6 - DNA, 19 bp, unidentified
  source 1..19: mol_type = other DNA
gcaccaatgtggagaccgt

SEQ ID NO: 7 - DNA, 19 bp, unidentified
  source 1..19: mol_type = other DNA
tataacaagcaaggaatgc

SEQ ID NO: 8 - DNA, 3687 bp, unidentified
  source 1..3687: mol_type = other DNA
atgagactgttcgtgagtgatggcgtcccgggttgcttgccggtgctggccgccgccgggagagcccggggcagagcagaggtgctcatcagcactgtaggcccggaagattgtgtggtcccgttcctgacccggcctaaggtccctgtcttgcagctggatagcggcaactacctcttctccactagtgcaatctgccgatatttttttttgttatctggctgggagcaagatgacctcactaaccagtggctggaatgggaagcgacagagctgcagccagctttgtctgctgccctgtactatttagtggtccaaggcaagaagggggaagatgttcttggttcagtgcggagagccctgactcacattgaccacagcttgagtcgtcagaactgtcctttcctggctggggagacagaatctctagccgacattgttttgtggggagccctatacccattactgcaagatcccgcctacctccctgaggagctgagtgccctgcacagctggttccagacactgagtacccaggaaccatgtcagcgagctgcagagactgtactgaaacagcaaggtgtcctggctctccggccttacctccaaaagcagccccagcccagccccgctgagggaagggctgtcaccaatgagcctgaggaggaggagctggctaccctatctgaggaggagattgctatggctgttactgcttgggagaagggcctagaaagtttgcccccgctgcggccccagcagaatccagtgttgcctgtggctggagaaaggaatgtgctcatcaccagtgccctcccttacgtcaacaatgtcccccaccttgggaacatcattggttgtgtgctcagtgccgatgtctttgccaggtactctcgcctccgccagtggaacaccctctatctgtgtgggacagatgagtatggtacagcaacagagaccaaggctctggaggagggactaaccccccaggagatctgcgacaagtaccacatcatccatgctgacatctaccgctggtttaacatttcgtttgatatttttggtcgcaccaccactccacagcagaccaaaatcacccaggacattttccagcagttgctgaaacgaggttttgtgctgcaagatactgtggagcaactgcgatgtgagcactgtgctcgcttcctggctgaccgcttcgtggagggcgtgtgtcccttctgtggctatgaggaggctcggggtgaccagtgtgacaagtgtggcaagctcatcaatgctgtcgagcttaagaaaatggagctggcgctggtgcctgtgagcgcccacggcaacttctatgagggggactgctacgtcatcctctcgacccggagagtggccagtctcctatcccaggacatccacttctggatcgggaaggactcctcccaggatgagcaaagctgcgcagccatatataccacacagctggacgactacctgggaggcagccctgtgcagcaccgagaggtccagtaccatgagtcagacactttccgtggctacttcaagcagggcatcatctacaagcaggggggtgtcgcctctgggatgaagcacgtggagaccaatacctacgacgtgaagcggctgctacatgtgaaagggaaaagaaacatcagggctaccgaggtggaaatgagctgggacagtttcaaccgaggtgatgtcttcttgctggaccttgggaaagtcatcatccaatggaatggcccagagagcaacagtggggagcgcctgaaggctatgcttctggcaaaggatattcgagacagggagcgagggggccgtgctaaaataggagtgatcgagggagacaaggaggcagccagcccagagctgatgaaggtccttcaggacacccttggccgacgctccattatcaagcctacagtccctgatgagatcatagatcagaagcagaaatcaactatcatgttgtatcatatctcagattcagctgggcagctggcagtcacagaggtagcaacaaggcctctggtccaggacttactgaaccatgatgactgctacatcctggaccaaagtggaaccaaaatctacgtgtggaaaggaaaaggagccacaaaggctgaaaaacaggcagccatgtctaaagcgctgggcttcatcaagatgaagagctaccccagcagcaccaatgtggagaccgtcaacgatggtgctgagtcggccatgttcaagcagctgttccagaagtggtcagtaaaggaccagaccatgggcctggggaaaacgttcagcattggtaaaattgctaaagttttccaggataaatttgatgtgactctgctacacaccaagccagaggtagctgcccaggaaagaatggtcgatgatggcaacggaaaagttgaggtctggagaattgagaacctggagctggtccctgtggagtatcaatggtatggcttcttttatgggggagactgttatctggtcctctacacatacgaggtaaatgggaagccacatcacatcttgtacatctggcagggccgccacgcctcacaggatgagctggcagcctcagcataccaggcagtggaggtggatcggcagtttgatggggctgctgtgcaggttcgagtcaggatgggaacggagccacgccacttcatggccatcttcaaagggaagctagttatctttgagggtgggacttccaggaagggaaatgccgagcctgaccctccagtaagactcttccaaattcatggaaatgacaaatctaacaccaaagcagtggaagttccagcctttgcctcctccctaaactccaatgatgtctttctgctgcgaactcaggcagagcactacctgtggtatggcaaggggtctagtggggatgagcgggcaatggctaaggagctggccagccttctctgtgatggcagcgagaacactgtggccgagggccaggagccagccgagttctgggacctactgggagggaaaactccctatgccaatgataaaagacttcagcaggaaatcctagatgtccagtctcgtctctttgaatgttccaataagaccggccaattcgttgtcactgagatcacagacttcacccaggatgacctgaaccctactgacgtgatgctcctagatacctgggaccaggtgttcttgtggattggggctgaggccaatgccacggagaaggagagtgcccttgccacagcacagcagtacctgcacactcaccccagcggccgagatcccgacacaccaatcctgatcattaagcaggggtttgagcctcccatcttcacaggctggttcctagcctgggaccctaacatttggagtgcaggaaaaacatatgaacaattaaaagaagagctgggagatgctgctgctatcatgcgaatcactgctgacatgaagaatgcaaccctctccctgaattctaatgacagtgagccaaaatattaccctatagcagttctgttgaaaaaccagaatcaggagctgcctgaggatgtaaaccctgccaaaaaggagaattacctctctgaacaggactttgtgtctgtgtttggcatcacaagagggcaatttgcagctctgcctggctggaaacagctccaaatgaagaaagaaaaggggcttttctaa

SEQ ID NO: 9 - DNA, 2460 bp, unidentified
  source 1..2460: mol_type = other DNA
atgcctctgaccagtgccttcagggctgtggacaacgaccctgggatcattgtctggagaatagagaaaatggagctggcgctggtgcctgtgagcgcccacggcaacttctatgagggggactgctacgtcatcctctcgacccggagagtggccagtctcctatcccaggacatccacttctggatcgggaaggactcctcccaggatgagcaaagctgcgcagccatatataccacacagctggacgactacctgggaggcagccctgtgcagcaccgagaggtccagtaccatgagtcagacactttccgtggctacttcaagcagggcatcatctacaagcaggggggtgtcgcctctgggatgaagcacgtggagaccaatacctacgacgtgaagcggctgctacatgtgaaagggaaaagaaacatcagggctaccgaggtggaaatgagctgggacagtttcaaccgaggtgatgtcttcttgctggaccttgggaaagtcatcatccaatggaatggcccagagagcaacagtggggagcgcctgaaggctatgcttctggcaaaggatattcgagacagggagcgagggggccgtgctaaaataggagtgatcgagggagacaaggaggcagccagcccagagctgatgaaggtccttcaggacacccttggccgacgctccattatcaagcctacagtccctgatgagatcatagatcagaagcagaaatcaactatcatgttgtatcatatctcagattcagctgggcagctggcagtcacagaggtagcaacaaggcctctggtccaggacttactgaaccatgatgactgctacatcctggaccaaagtggaaccaaaatctacgtgtggaaaggaaaaggagccacaaaggctgaaaaacaggcagccatgtctaaagcgctgggcttcatcaagatgaagagctaccccagcagcaccaatgtggagaccgtcaacgatggtgctgagtcggccatgttcaagcagctgttccagaagtggtcagtaaaggaccagaccatgggcctggggaaaacgttcagcattggtaaaattgctaaagttttccaggataaatttgatgtgactctgctacacaccaagccagaggtagctgcccaggaaagaatggtcgatgatggcaacggaaaagttgaggtctggagaattgagaacctggagctggtccctgtggagtatcaatggtatggcttcttttatgggggagactgttatctggtcctctacacatacgaggtaaatgggaagccacatcacatcttgtacatctggcagggccgccacgcctcacaggatgagctggcagcctcagcataccaggcagtggaggtggatcggcagtttgatggggctgctgtgcaggttcgagtcaggatgggaacggagccacgccacttcatggccatcttcaaagggaagctagttatctttgagggtgggacttccaggaagggaaatgccgagcctgaccctccagtaagactcttccaaattcatggaaatgacaaatctaacaccaaagcagtggaagttccagcctttgcctcctccctaaactccaatgatgtctttctgctgcgaactcaggcagagcactacctgtggtatggcaaggggtctagtggggatgagcgggcaatggctaaggagctggccagccttctctgtgatggcagcgagaacactgtggccgagggccaggagccagccgagttctgggacctactgggagggaaaactccctatgccaatgataaaagacttcagcaggaaatcctagatgtccagtctcgtctctttgaatgttccaataagaccggccaattcgttgtcactgagatcacagacttcacccaggatgacctgaaccctactgacgtgatgctcctagatacctgggaccaggtgttcttgtggattggggctgaggccaatgccacggagaaggagagtgcccttgccacagcacagcagtacctgcacactcaccccagcggccgagatcccgacacaccaatcctgatcattaagcaggggtttgagcctcccatcttcacaggctggttcctagcctgggaccctaacatttggagtgcaggaaaaacatatgaacaattaaaagaagagctgggagatgctgctgctatcatgcgaatcactgctgacatgaagaatgcaaccctctccctgaattctaatgacagtgagccaaaatattaccctatagcagttctgttgaaaaaccagaatcaggagctgcctgaggatgtaaaccctgccaaaaaggagaattacctctctgaacaggactttgtgtctgtgtttggcatcacaagagggcaatttgcagctctgcctggctggaaacagctccaaatgaagaaagaaaaggggcttttctaa

SEQ ID NO: 10 - DNA, 3094 bp, unidentified
  source 1..3094: mol_type = other DNA
cccttctgtggctatgaggaggctcggggtgaccagtgtgacaagtgtggcaagctcatcaatgctgtcgagcttaaggtaagaggagggtctccatgggagcccggaaggagacagtccttattcttaagggacgcccttcctgtcccatttaggatttttattttagcattctagacctttaaccagtggccctttctgccccatctcagcaactcgtcatttagttcatttgaaaaatactccctgagcacttactatgtgctgagcactgtgctaagaaacagacaagttgagtttatatttcccaacctttggtcttgcatcacaccacctgatctacatttgtttagatccttctctcttctttaaatatcttctcttctagccatttgttaatttatttcttcattcatttattcaacatacattttttgaatccttagaataggccaattacaattctgcgtgctgggggtatggcaatgaataagacagattttatttagtatggtcaggagaccaacactaactaaggaaatgatatagaataaactgccaggaaaatgtgacagagagtgacttctgggggaagatctaacttaggtagatagggaaggcctttcagaggcagtaacatttgaactgagagtttaaccaaagaattaggagcaagccaggcaataagaggttaagaatgttctaagcggaaggactaggaaatgcaaaggccatgaggcagctaagagctgaaaaggcagaattggcaaggccagtatagcactagcacagtgagtgaggaggagggtagactgagatgaggtgagagagatggcaggggccagatcaccagggcctgtgaagccattgtgggaatttagattttattctggtgaatgacaagctgctctgtggaaaatggatggcagggaagcaagaaataaagtatgttgaaggggctgggcattggtggctcatacctataatcccagcactttgggaggccaagctgggtggatcacttgaagccacgagtttgagaccagcctggcctgtaacatggcaaccctgtctctactgaaaatacagaaatggctaggtgcggtggctcacgcctgtaatcccagcactttgggaggctgaggtgggcagatcacttgaggtcaggagtttgagaccagcctggccaacatggtgaaaccctgtctctactaaaaaaatacaaaaattagctgggtgtggtggcggacccctgtaatccccgcttcttgggaggctgaagcttgagaatcacttgaacctgggaggcagaggttgcagtgagctgagattgcgccactgcattccagcctgggttacaaagtaagactctgtctaaaaaaaaaaaaaaaccacaaaaattatccaggccgtggtggcgcacacctgtaatcccagctacttgggaggctgaggcagaagaatttcttgaacctgggaagcggaggttgcagtgagccaagattgccactgcattccagcctgggtgacagagcaagactgttccccctccaccaaaaaaaaaagtatggagaaggatttggaggcaacaaggctacaacccagaaatgggataatggtggcttgcactaggttagtagcactagaaatggaaaagaaaaaatagacttgaggtatatttttggaggtaaactccacggagttggatagactggaatttgggatggtcaggaaaaagatgccttgtaagtttttggcctgagtagatggtagtaccatctgtgaacgtgagtgaggagaacaggctttcaggtgggatgcagccatactgtttgggtttgttaagcttgagatgcctgttagatagccaggcagagaagtcaggtagacagttggatattaggcatatggagttcaggagagaggtcctgaggaattataatttctcaccttcaaaaactcagatcaaaactcacttggggccaggtgtggtggctcacgcttgtaatcccaggactttgggaagctgagggaccaaggttcagatccggggtgatgcaagcagagctcacagaagccagagggatggccacgttaaccctgtggctggcagcctcacatcctgtaaggtcattgagaacatctcccctccttctccccaccacctgcaccgaatgacagagaggagagggaggctcatggggaagggaggtgggaggggatgtagcctatacccaccccttgtccttccagaaaatggagctggcgctggtgcctgtgagcgcccacggcaacttctatgagggggactgctacgtcatcctctcggtgagcaccctcctccccactcctgtgagcctctcccctggggccagttcttgggctgtactgggagatgtgctggtgagggaaggtagtcagtccctggtatcggtctcaatggttcctcagtgctatgcagagcagacctccagtctgactatcaaaggccatcattaagcctgggtttttaatgctgcataagaggatgggggtaggaccctcagtctgaacctcagtgagccacccacaagcagcacttctcccgggcctaggccccacattaaagtggtgtggaagaaggcctgataaatgcaaggcctgtctctagcctcaaagtgctattctcatggaggagactcacatgaacggtgctgacagggtcgtgcaggtgtacagatgagcactgtgccagctgtgtgagtgcaagggaccaaggggacctgtgaggtaagcctgcaacccggctgggaaaagatccccgttggtatctatttatttggggaacaacaagctgtcaactttccccaaatgcggggaggaatttgggctgccaaagtttatgccatgatcgcaggtctgctcgggggccagggcagctcccacatccactgcaggagagagggccctcagtctgccaagggcatgcacatgtgctgcattctccccataacaggggccagcagcaaaggggaggtgagctggcctctgacctgatctctctgactacctcccagacccggagagtggccagtctcctatcccaggacatcca

SEQ ID NO: 11 - RNA, 191 nt, unidentified
  source 1..191: mol_type = other RNA
cccttctgtggctatgaggaggctcggggtgaccagtgtgacaagtgtggcaagctcatcaatgctgtcgagcttaagaaaatggagctggcgctggtgcctgtgagcgcccacggcaacttctatgagggggactgctacgtcatcctctcgacccggagagtggccagtctcctatcccaggacatcca

SEQ ID NO: 12 - RNA, 23 nt, unidentified
  source 1..23: mol_type = other RNA
ctggagaatagagaaaatggagc

SEQ ID NO: 13 - RNA, 23 nt, unidentified
  source 1..23: mol_type = other RNA
tacatgtgaaagggaaaagaaac

SEQ ID NO: 14 - RNA, 23 nt, unidentified
  source 1..23: mol_type = other RNA
tagatcagaagcagaaatcaact

SEQ ID NO: 15 - RNA, 22 nt, unidentified
  source 1..22: mol_type = other RNA
agcagaaatcaactatcatgtt

SEQ ID NO: 16 - RNA, 23 nt, unidentified
  source 1..23: mol_type = other RNA
atcaactatcatgttgtatcata

SEQ ID NO: 17 - RNA, 23 nt, unidentified
  source 1..23: mol_type = other RNA
aactatcatgttgtatcatatct

SEQ ID NO: 18 - RNA, 23 nt, unidentified
  source 1..23: mol_type = other RNA
atcatgttgtatcatatctcaga

SEQ ID NO: 19 - RNA, 23 nt, unidentified
  source 1..23: mol_type = other RNA
ttgtatcatatctcagattcagc

SEQ ID NO: 20 - RNA, 23 nt, unidentified
  source 1..23: mol_type = other RNA
agccacaaaggctgaaaaacagg

SEQ ID NO: 21 - RNA, 23 nt, unidentified
  source 1..23: mol_type = other RNA
agcattggtaaaattgctaaagt

SEQ ID NO: 22 - RNA, 23 nt, unidentified
  source 1..23: mol_type = other RNA
ttggtaaaattgctaaagttttc

SEQ ID NO: 23 - RNA, 23 nt, unidentified
  source 1..23: mol_type = other RNA
tggtaaaattgctaaagttttcc

SEQ ID NO: 24 - RNA, 23 nt, unidentified
  source 1..23: mol_type = other RNA
atcaatggtatggcttcttttat

SEQ ID NO: 25 - RNA, 23 nt, unidentified
  source 1..23: mol_type = other RNA
ctctacacatacgaggtaaatgg

SEQ ID NO: 26 - RNA, 23 nt, unidentified
  source 1..23: mol_type = other RNA
gccacttcatggccatcttcaaa

SEQ ID NO: 27 - RNA, 23 nt, unidentified
  source 1..23: mol_type = other RNA
tccagtaagactcttccaaattc

SEQ ID NO: 28 - RNA, 23 nt, unidentified
  source 1..23: mol_type = other RNA
ctcttccaaattcatggaaatga

SEQ ID NO: 29 - RNA, 22 nt, unidentified
  source 1..22: mol_type = other RNA
tccaaattcatggaaatgacaa

SEQ ID NO: 30 - RNA, 23 nt, unidentified
  source 1..23: mol_type = other RNA
ttcatggaaatgacaaatctaac

SEQ ID NO: 31 - RNA, 23 nt, unidentified
  source 1..23: mol_type = other RNA
atggaaatgacaaatctaacacc

SEQ ID NO: 32 - RNA, 23 nt, unidentified
  source 1..23: mol_type = other RNA
ctccctaaactccaatgatgtct

SEQ ID NO: 33 - RNA, 23 nt, unidentified
  source 1..23: mol_type = other RNA
gtctctttgaatgttccaataag

SEQ ID NO: 34 - RNA, 23 nt, unidentified
  source 1..23: mol_type = other RNA
tgcaggaaaaacatatgaacaat

SEQ ID NO: 35 - RNA, 23 nt, unidentified
  source 1..23: mol_type = other RNA
caggaaaaacatatgaacaatta

SEQ ID NO: 36 - RNA, 23 nt, unidentified
  source 1..23: mol_type = other RNA
aggaaaaacatatgaacaattaa

SEQ ID NO: 37 - RNA, 23 nt, unidentified
  source 1..23: mol_type = other RNA
aagcaagaaggcctatacctatt

SEQ ID NO: 38 - RNA, 23 nt, unidentified
  source 1..23: mol_type = other RNA
tgccaaaaaggagaattacctct

SEQ ID NO: 39 - RNA, 23 nt, unidentified
  source 1..23: mol_type = other RNA
ctggaaacagctccaaatgaaga

SEQ ID NO: 40 - RNA, 23 nt, unidentified
  source 1..23: mol_type = other RNA
agctccaaatgaagaaagaaaag

SEQ ID NO: 41 - RNA, 23 nt, unidentified
  source 1..23: mol_type = other RNA
aagcaagaaggcctatacctatt

SEQ ID NO: 42 - RNA, 23 nt, unidentified
  source 1..23: mol_type = other RNA
gagcagatagtgccaatatcagg

SEQ ID NO: 43 - RNA, 23 nt, unidentified
  source 1..23: mol_type = other RNA
ggcatgttctccattttttctca

SEQ ID NO: 44 - RNA, 23 nt, unidentified
  source 1..23: mol_type = other RNA
tgcattccttgcttgttatatac

SEQ ID NO: 45 - RNA, 23 nt, unidentified
  source 1..23: mol_type = other RNA
gtgccaatatcaggaaataattt

SEQ ID NO: 46 - RNA, 23 nt, unidentified
  source 1..23: mol_type = other RNA
tgccaatatcaggaaataattta

SEQ ID NO: 47 - RNA, 23 nt, unidentified
  source 1..23: mol_type = other RNA
gccaatatcaggaaataatttat

SEQ ID NO: 48 - RNA, 23 nt, unidentified
  source 1..23: mol_type = other RNA
gcctgacattcagctacttaatt

SEQ ID NO: 49 - RNA, 23 nt, unidentified
  source 1..23: mol_type = other RNA
ctgacattcagctacttaattta

SEQ ID NO: 50 - RNA, 23 nt, unidentified
  source 1..23: mol_type = other RNA
cagctacttaatttagatataat

SEQ ID NO: 51 - RNA, 23 nt, unidentified
  source 1..23: mol_type = other RNA
agctacttaatttagatataata

SEQ ID NO: 52 - RNA, 23 nt, unidentified
  source 1..23: mol_type = other RNA
ctgcaaatcacggcatgttctcc

SEQ ID NO: 53 - RNA, 23 nt, unidentified
  source 1..23: mol_type = other RNA
ttgcttgttatatacctaaaatg

SEQ ID NO: 54 - RNA, 23 nt, unidentified
  source 1..23: mol_type = other RNA
tgcttgttatatacctaaaatgt

SEQ ID NO: 55 - RNA, 23 nt, unidentified
  source 1..23: mol_type = other RNA
ttgttatatacctaaaatgttaa

SEQ ID NO: 56 - RNA, 23 nt, unidentified
  source 1..23: mol_type = other RNA
tacctaaaatgttaaccatatag

SEQ ID NO: 57 - RNA, 23 nt, unidentified
  source 1..23: mol_type = other RNA
acctaaaatgttaaccatatagt

SEQ ID NO: 58 - RNA, 23 nt, unidentified
  source 1..23: mol_type = other RNA
aagcactaaaactgcataaatct

SEQ ID NO: 59 - RNA, 23 nt, unidentified
  source 1..23: mol_type = other RNA
tgcataaatctggagaaatcaaa

SEQ ID NO: 60 - RNA, 23 nt, unidentified
  source 1..23: mol_type = other RNA
ctggagaaatcaaaagaaagaga

SEQ ID NO: 61 - RNA, 23 nt, unidentified
  source 1..23: mol_type = other RNA
gagaaatcaaaagaaagagaacc

SEQ ID NO: 62 - RNA, 23 nt, unidentified
  source 1..23: mol_type = other RNA
aagaaagagaaccaaaaaacaat

SEQ ID NO: 63 - RNA, 23 nt, unidentified
  source 1..23: mol_type = other RNA
aaccaaaaaacaatgcttaaaat

SEQ ID NO: 64 - RNA, 23 nt, unidentified
  source 1..23: mol_type = other RNA
accaaaaaacaatgcttaaaatg

SEQ ID NO: 65 - RNA, 23 nt, unidentified
  source 1..23: mol_type = other RNA
aacaatgcttaaaatgtttaata

SEQ ID NO: 66 - RNA, 23 nt, unidentified
  source 1..23: mol_type = other RNA
tgcttaaaatgtttaataacttt

SEQ ID NO: 67 - RNA, 23 nt, unidentified
  source 1..23: mol_type = other RNA
atgtttaataactttatgtttaa

SEQ ID NO: 68 - RNA, 23 nt, unidentified
  source 1..23: mol_type = other RNA
aactttatgtttaatattatacc

SEQ ID NO: 69 - RNA, 23 nt, unidentified
  source 1..23: mol_type = other RNA
acctacctttgttttcaatttta

SEQ ID NO: 70 - RNA, 23 nt, unidentified
  source 1..23: mol_type = other RNA
tacctttgttttcaattttaaga

SEQ ID NO: 71 - RNA, 23 nt, unidentified
  source 1..23: mol_type = other RNA
ttgttttcaattttaagatgatt

SEQ ID NO: 72 - RNA, 23 nt, unidentified
  source 1..23: mol_type = other RNA
ttcaattttaagatgattatttc

SEQ ID NO: 73 - RNA, 23 nt, unidentified
  source 1..23: mol_type = other RNA
atgattatttctaaaatctattt

SEQ ID NO: 74 - RNA, 23 nt, unidentified
  source 1..23: mol_type = other RNA
atctatttagcctgtaaatcatt

SEQ ID NO: 75 - RNA, 23 nt, unidentified
  source 1..23: mol_type = other RNA
gcctgtaaatcattgaaatcata

SEQ ID NO: 76 - RNA, 23 nt, unidentified
  source 1..23: mol_type = other RNA
ctgtaaatcattgaaatcatata

SEQ ID NO: 77 - RNA, 23 nt, unidentified
  source 1..23: mol_type = other RNA
atcattgaaatcatatatgcact

SEQ ID NO: 78 - RNA, 23 nt, unidentified
  source 1..23: mol_type = other RNA
tccaaatacccagatctgtaatg

SEQ ID NO: 79 - RNA, 23 nt, unidentified
  source 1..23: mol_type = other RNA
ctgtaatgtgtcaaagcattttt

SEQ ID NO: 80 - RNA, 23 nt, unidentified
  source 1..23: mol_type = other RNA
gtcaaagcatttttcacttttca

SEQ ID NO: 81 - RNA, 23 nt, unidentified
  source 1..23: mol_type = other RNA
aagcatttttcacttttcaaata

SEQ ID NO: 82 - RNA, 23 nt, unidentified
  source 1..23: mol_type = other RNA
agcatttttcacttttcaaataa

SEQ ID NO: 83 - RNA, 23 nt, unidentified
  source 1..23: mol_type = other RNA
ttcacttttcaaataaagatacc

SEQ ID NO: 84 - RNA, 23 nt, unidentified
  source 1..23: mol_type = other RNA
ttcaaataaagatacctataatg

SEQ ID NO: 85 - RNA, 19 nt, unidentified
  source 1..19: mol_type = other RNA
ccacacagctggacgacta

SEQ ID NO: 86 - RNA, 19 nt, unidentified
  source 1..19: mol_type = other RNA
gcttctggcaaaggatatt

SEQ ID NO: 87 - RNA, 19 nt, unidentified
  source 1..19: mol_type = other RNA
gcaaaggatattcgagaca

SEQ ID NO: 88 - RNA, 23 nt, unidentified
  source 1..23: mol_type = other RNA
atcaactatcatgttgtatcata

SEQ ID NO: 89 - RNA, 19 nt, unidentified
  source 1..19: mol_type = other RNA
gcaccaatgtggagaccgt

SEQ ID NO: 90 - RNA, 19 nt, unidentified
  source 1..19: mol_type = other RNA
ggaaagaatggtcgatgat

SEQ ID NO: 91 - RNA, 23 nt, unidentified
  source 1..23: mol_type = other RNA
aattcatggaaatgacaaatcta

SEQ ID NO: 92 - RNA, 19 nt, unidentified
  source 1..19: mol_type = other RNA
ttacctctctgaacaggac

SEQ ID NO: 93 - RNA, 22 nt, unidentified
  source 1..22: mol_type = other RNA
aacaggactttgtgtctgtgtt

SEQ ID NO: 94 - RNA, 19 nt, unidentified
  source 1..19: mol_type = other RNA
gcaagaaggcctataccta

SEQ ID NO: 95 - RNA, 19 nt, unidentified
  source 1..19: mol_type = other RNA
gcattccttgcttgttata

SEQ ID NO: 96 - RNA, 19 nt, unidentified
  source 1..19: mol_type = other RNA
tattataccaggacctacc

SEQ ID NO: 97 - RNA, 19 nt, unidentified
  source 1..19: mol_type = other RNA
gagcttaagaaaatggagc

SEQ ID NO: 98 - RNA, 58 nt, unidentified
  source 1..58: mol_type = other RNA
gatccgcttctggcaaaggatattttcaagagaaatatcctttgccagaagctttttg

SEQ ID NO: 99 - RNA, 58 nt, unidentified
  source 1..58: mol_type = other RNA
aattcaaaaagcttctggcaaaggatatttctcttgaaaatatcctttgccagaagcg

SEQ ID NO: 100 - RNA, 58 nt, unidentified
  source 1..58: mol_type = other RNA
gatccgcattccttgcttgttatattcaagagatataacaagcaaggaatgctttttg

SEQ ID NO: 101 - RNA, 58 nt, unidentified
  source 1..58: mol_type = other RNA
aattcaaaaagcattccttgcttgttatatctcttgaatataacaagcaaggaatgcg

SEQ ID NO: 102 - RNA, 69 nt, unidentified
  source 1..69: mol_type = other RNA
gcgcatcaactatcatgttgtatcatagtgaagccacagatgtatgatacaacatgatagttgatttgc

SEQ ID NO: 103 - RNA, 69 nt, unidentified
  source 1..69: mol_type = other RNA
gcaaatcaactatcatgttgtatcatacatctgtggcttcactatgatacaacatgatagttgatgcgc

SEQ ID NO: 104 - RNA, 69 nt, unidentified
  source 1..69: mol_type = other RNA
gcgcttcatggaaatgacaaatctatagtgaagccacagatgtatagatttgtcatttccatgaattgc

SEQ ID NO: 105 - RNA, 69 nt, unidentified
  source 1..69: mol_type = other RNA
gcaattcatggaaatgacaaatctatacatctgtggcttcactatagatttgtcatttccatgaagcgc

SEQ ID NO: 106 - RNA, 69 nt, unidentified
  source 1..69: mol_type = other RNA
gcgccaggactttgtgtctgtgttatagtgaagccacagatgtataacacagacacaaagtcctgttgc

SEQ ID NO: 107 - RNA, 69 nt, unidentified
  source 1..69: mol_type = other RNA
gcaacaggactttgtgtctgtgttatacatctgtggcttcactataacacagacacaaagtcctggcgc

SEQ ID NO: 108 - RNA, 25 nt, unidentified
  source 1..25: mol_type = other RNA
caccgtagcagccgcttcacgtcgt

SEQ ID NO: 109 - RNA, 25 nt, unidentified
  source 1..25: mol_type = other RNA
aaacacgacgtgaagcggctgctac

SEQ ID NO: 110 - RNA, 25 nt, unidentified
  source 1..25: mol_type = other RNA
caccgagctgggacagtttcaaccg

SEQ ID NO: 111 - RNA, 25 nt, unidentified
  source 1..25: mol_type = other RNA
aaaccggttgaaactgtcccagctc

SEQ ID NO: 112 - RNA, 25 nt, unidentified
  source 1..25: mol_type = other RNA
caccgaagaaacatcagggctaccg

SEQ ID NO: 113 - RNA, 25 nt, unidentified
  source 1..25: mol_type = other RNA
aaaccggtagccctgatgtttcttc

SEQ ID NO: 114 - RNA, 25 nt, unidentified
  source 1..25: mol_type = other RNA
caccgcaacagtggggagcgcctga

SEQ ID NO: 115 - RNA, 25 nt, unidentified
  source 1..25: mol_type = other RNA
aaactcaggcgctccccactgttgc

SEQ ID NO: 116 - RNA, 19 nt, unidentified
  source 1..19: mol_type = other RNA
gcttctggcaaaggatatt

SEQ ID NO: 117 - RNA, 19 nt, unidentified
  source 1..19: mol_type = other RNA
ccacacagctggacgacta

SEQ ID NO: 118 - RNA, 19 nt, unidentified
  source 1..19: mol_type = other RNA
gcaaaggatattcgagaca

SEQ ID NO: 119 - RNA, 19 nt, unidentified
  source 1..19: mol_type = other RNA
gcaccaatgtggagaccgt

SEQ ID NO: 120 - RNA, 19 nt, unidentified
  source 1..19: mol_type = other RNA
ggaaagaatggtcgatgat

SEQ ID NO: 121 - RNA, 19 nt, unidentified
  source 1..19: mol_type = other RNA
gcattccttgcttgttata

SEQ ID NO: 122 - RNA, 19 nt, unidentified
  source 1..19: mol_type = other RNA
gagcttaagaaaatggagc

SEQ ID NO: 123 - RNA, 19 nt, unidentified
  source 1..19: mol_type = other RNA
agctgcgcagccatatata

SEQ ID NO: 124 - RNA, 19 nt, unidentified
  source 1..19: mol_type = other RNA
tcactgctgacatgaagaa

SEQ ID NO: 125 - RNA, 19 nt, unidentified
  source 1..19: mol_type = other RNA
aacaggcagccatgtctaa

SEQ ID NO: 126 - RNA, 19 nt, unidentified
  source 1..19: mol_type = other RNA
gttgaggtctggagaattg

SEQ ID NO: 127 - RNA, 19 nt, unidentified
  source 1..19: mol_type = other RNA
ggagaattacctctctgaa

SEQ ID NO: 128 - RNA, 19 nt, unidentified
  source 1..19: mol_type = other RNA
gcaagaaggcctataccta

SEQ ID NO: 129 - RNA, 19 nt, unidentified
  source 1..19: mol_type = other RNA
gaaggcctatacctattgc

SEQ ID NO: 130 - RNA, 19 nt, unidentified
  source 1..19: mol_type = other RNA
agagcagatagtgccaata

SEQ ID NO: 131 - RNA, 19 nt, unidentified
  source 1..19: mol_type = other RNA
gctgcgcagccatatatac

SEQ ID NO: 132 - RNA, 19 nt, unidentified
  source 1..19: mol_type = other RNA
agcacgtggagaccaatac

SEQ ID NO: 133 - RNA, 19 nt, unidentified
  source 1..19: mol_type = other RNA
gcagaaatcaactatcatg

SEQ ID NO: 134 - RNA, 19 nt, unidentified
  source 1..19: mol_type = other RNA
cgtctttagttgatagtac

SEQ ID NO: 135 - RNA, 19 nt, unidentified
  source 1..19: mol_type = other RNA
caggactttgtgtctgtgt

SEQ ID NO: 136 - RNA, 19 nt, unidentified
  source 1..19: mol_type = other RNA
gtcctgaaacacagacaca

SEQ ID NO: 137 - RNA, 19 nt, unidentified
  source 1..19: mol_type = other RNA
gcaagaaggcctataccta

SEQ ID NO: 138 - RNA, 19 nt, unidentified
  source 1..19: mol_type = other RNA
cgttcttccggatatggat

SEQ ID NO: 139 - RNA, 19 nt, unidentified
  source 1..19: mol_type = other RNA
atcacggcatgttctccat

SEQ ID NO: 140 - RNA, 19 nt, unidentified
  source 1..19: mol_type = other RNA
tagtgccgtacaagaggta

SEQ ID NO: 141 - RNA, 19 nt, unidentified
  source 1..19: mol_type = other RNA
ttacctctctgaacaggac

SEQ ID NO: 142 - RNA, 19 nt, unidentified
  source 1..19: mol_type = other RNA
aatggagagacttgtcctg

SEQ ID NO: 143 - RNA, 19 nt, unidentified
  source 1..19: mol_type = other RNA
tattataccaggacctacc

SEQ ID NO: 144 - RNA, 19 nt, unidentified
  source 1..19: mol_type = other RNA
ataatatggtcctggatgg

SEQ ID NO: 145 - RNA, 23 nt, unidentified
  source 1..23: mol_type = mRNA
aaagctgcgcagccatatatacc

SEQ ID NO: 146 - RNA, 23 nt, unidentified
  source 1..23: mol_type = mRNA
aatcactgctgacatgaagaatg

SEQ ID NO: 147 - RNA, 23 nt, unidentified
  source 1..23: mol_type = mRNA
aaaacaggcagccatgtctaaag

SEQ ID NO: 148 - RNA, 23 nt, unidentified
  source 1..23: mol_type = mRNA
aagttgaggtctggagaattgag

SEQ ID NO: 149 - RNA, 23 nt, unidentified
  source 1..23: mol_type = mRNA
aaggagaattacctctctgaaca

SEQ ID NO: 150 - RNA, 23 nt, unidentified
  source 1..23: mol_type = mRNA
aagcaagaaggcctatacctatt

SEQ ID NO: 151 - RNA, 23 nt, unidentified
  source 1..23: mol_type = mRNA
aagaaggcctatacctattgcaa

SEQ ID NO: 152 - RNA, 23 nt, unidentified
  source 1..23: mol_type = other RNA
aaagagcagatagtgccaatatc

SEQ ID NO: 153 - RNA, 23 nt, unidentified
  source 1..23: mol_type = mRNA
aagctgcgcagccatatatacca

SEQ ID NO: 154 - RNA, 23 nt, unidentified
  source 1..23: mol_type = mRNA
gaagcacgtggagaccaatacct

SEQ ID NO: 155 - RNA, 19 nt, unidentified
  source 1..19: mol_type = mRNA
gcagaaatcaactatcatg

SEQ ID NO: 156 - RNA, 19 nt, unidentified
  source 1..19: mol_type = mRNA
caggactttgtgtctgtgt

SEQ ID NO: 157 - RNA, 19 nt, unidentified
  source 1..19: mol_type = mRNA
gcaagaaggcctataccta

SEQ ID NO: 158 - RNA, 19 nt, unidentified
  source 1..19: mol_type = mRNA
atcacggcatgttctccat

SEQ ID NO: 159 - RNA, 19 nt, unidentified
  source 1..19: mol_type = mRNA
ttacctctctgaacaggac

SEQ ID NO: 160 - RNA, 19 nt, unidentified
  source 1..19: mol_type = mRNA
tattataccaggacctacc

(19) United States
(12) Patent Application Publication
(10) Pub. No.: US 2023/0364087 A1
(43) Pub. Date: Nov. 16, 2023

(54) THERAPEUTIC TARGETING OF ACTIVATED AVIL-INDUCED SARCOMAS

(71) Applicant: University of Virginia Patent Foundation, Charlottesville, VA (US)

(72) Inventors: Hui LI, Keswick, VA (US); Zhongqiu XIE, Charlottesville, VA (US)

(21) Appl. No.: 18/306,680

(22) Filed: Apr. 25, 2023

Related U.S. Application Data
(63) Continuation-in-part of application No. 17/809,145, filed on Jun. 27, 2022.
Application No. 17/809,145 is a continuation of application No. 16/759,210, filed on Apr. 24, 2020, now Pat. No. 11,369,608.
(60) Provisional application No. 63/334,910, filed on Apr. 26, 2022.
(60) Provisional application No. 62/577,749, filed on Oct. 27, 2017.

(51) Int. Cl. A61K 31/506; A61K 31/40; A61P 35/04; A61K 31/454; A61K 45/06

(52) U.S. Cl. CPC A61K 31/506; A61K 31/40; A61K 31/454; A61P 35/04; A61K 45/06

(57) ABSTRACT

Disclosed herein are silencing oligonucleotides useful to regulate, limit, or inhibit the expression of AVIL (advillin). Also disclosed herein are methods for treating disorders associated with AVIL dysregulation using same. In some embodiments, the method involves treating a cancer, such as glioblastomas, rhabdosarcomas, gliomas, lung cancer, bladder cancer, or renal cancer.

### CROSS-REFERENCE TO RELATED APPLICATIONS

This application is a continuation-in-part of copending Application Serial No. 17/809,145, filed Jun. 27, 2022, which is a continuation application of U.S. Nonprovisional Application No. 16/759,210 filed on Apr. 24, 2020, now U.S. Pat. No. 11,369,608, which is a U.S. National Phase Application under 35 USC § 371 of international application no. PCT/US2018/057697, filed Oct. 26, 2018, which claims the benefit of U.S. Provisional Application No. 62/577,749, filed on Oct. 27, 2017. This application also claims benefit of U.S. Provisional Application No. 63/334,910, filed Apr. 26, 2022. Each application is incorporated herein by reference in its entirety.

### STATEMENT REGARDING FEDERALLY SPONSORED RESEARCH OR

### DEVELOPMENT

This invention was made with Government Support under Grant No. CA240601 awarded by the National Institutes of Health. The Government has certain rights in the invention.

### SEQUENCE LISTING

This application contains a sequence listing filed in ST.26 format entitled “222117-1062 Sequence Listing” created on Apr. 25, 2023. The content of the sequence listing is incorporated herein in its entirety.

### BACKGROUND

Rhabdomyosarcoma (RMS) is one of the most common pediatric soft-tissue cancer. Previously, we discovered a gene fusion, MARS-AVIL formed by chromosomal inversion in RMS. Suspecting that forming a fusion with a housekeeping gene may be one of the mechanisms to dysregulate an oncogene, we investigated AVIL expression and its role in RMS. We first showed that MARS-AVIL translates into an in-frame fusion protein, which is critical for the RMS cell tumorigenesis. Besides forming a gene fusion with the housekeeping gene, MARS, the AVIL locus is often amplified, and its RNA and protein expression are overexpressed in the majority of RMSs. Tumors with AVIL dysregulation exhibit evidence of oncogene addiction: silencing MARS-AVIL in cells harboring the fusion, or silencing AVIL in cells with AVIL overexpression, nearly eradicated the cells in culture, as well as inhibited in vivo xenograft growth in mice. Conversely, gain-of-function manipulations of AVIL led to increased cell growth and migration, enhanced foci formation in mouse fibroblasts, and most importantly transformed mesenchymal stem cells in vitro and in vivo. Mechanistically, AVIL seems to serve as a converging node functioning upstream of two oncogenic pathways, PAX3-FOXO1 and RAS, thus connecting two types of RMS associated with these pathways. Interestingly, AVIL is overexpressed in other sarcoma cells as well, and its expression correlates with clinical outcomes, with higher levels of AVIL expression being associated with worse prognosis. AVIL is a bona fide oncogene in RMS, and RMS cells are addicted to its activity.

### SUMMARY

In accordance with the purpose(s) of the invention, as embodied and broadly described herein, the invention, in one aspect, relates to silencing oligonucleotides useful to regulate, limit, or inhibit the expression of AVIL (advillin), methods of making same, pharmaceutical compositions comprising same, and methods of treating disorders associated with AVIL dysregulation using same. In aspects, the disclosed silencing oligonucleotides, compositions and methods are useful for treating disorders or diseases in which the regulation, limitation, or inhibition of the expression of AVIL can be clinically useful, such as, for example, the treatment of cancer.

Also disclosed are pharmaceutical compositions comprising a therapeutically effective amount of one or more disclosed silencing oligonucleotide.

Also disclosed are methods for the treatment of an oncological disorder or disease associated with dysregulation of AVIL in a mammal comprising the step of administering to the mammal a therapeutically effective amount of at least one disclosed silencing oligonucleotide.

Also disclosed are methods for regulating, limiting, or inhibiting AVIL expression in a mammal comprising the step of administering to the mammal a therapeutically effective amount of at least one disclosed silencing oligonucleotide.

Also disclosed are methods for regulating, limiting, or inhibiting AVIL expression in at least one cell, comprising the step of contacting the cell with an effective amount of at least one disclosed silencing oligonucleotide.

Also disclosed are kits comprising at least one disclosed silencing oligonucleotide and one or more of: (a) at least one agent known to regulate, limit, or inhibit AVIL expression; (b) at least one agent known to treat cancer; and (c) instructions for treating cancer and/or for administering the compound in connection with cancer therapy.

The details of one or more embodiments of the invention are set forth in the accompanying drawings and the description below. Other features, objects, and advantages of the invention will be apparent from the description and drawings, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A to 1I show MARS-AVIL fusion in RMS. FIG. 1A shows the fusion is composed of the first ten exons of MARS and the last 15 exons of AVIL. The dotted line indicates the junction site. FIG. 1B shows long-range PCR was used to amplify genomic DNA fragment spanning exon10 of MARS and exon2 of AVIL in RH30 cells, but not in RH18 or MSC cells. FIG. 1C is a Western blot using MARS antibody identifying both MARS and MARS-AVIL in RH30 cells. 293T transfected with Myc tagged MARS-AVIL was included as a control. The band corresponding to MARS-AVIL was further confirmed by siAVIL1 silencing the fusion. FIGS. 1D-F and G-I show stable RD (FIG. 1D) and RH18 (FIG. 1G) cells expressing MARS-AVIL to a similar level in RH30. FIGS. 1E and H show cell proliferation measured by MTT was enhanced in RD (FIG. 1E) and RH18 (FIG. 1H) cells stably expressing MARS-AVIL. FIGS. 1F and 1I show cell motility measured by wound healing were enhanced in RD (FIG. 1F) and RH18 (FIG. 1I) cells stably expressing MARS-AVIL. Data are presented as mean values ± SD in FIGS. 1D to 1I. P value was calculated by standard two-tailed t-test. *p < 0.05, **p < 0.01, ***p < 0.001.

FIGS. 2A to 2Q show MARS-AVIL is necessary for tumorigenesis in RH30 cells. FIG. 2A shows RH30 cells transfected with siAVIL1, but not siAVIL2, resulted in a sub G1 peak by flow cytometry. FIG. 2B is a Western blot using PARP and cleaved Caspase3 antibodies detected cleaved PARP and Caspase3 in RH30 cells transfected with siAVIL1, but not siAVIL2. FIG. 2C shows crystal violet staining showed that most RH30 cells died when transfected with siAVIL1, but not siAVIL2. FIG. 2D shows RH30 cells with siAVIL1, but not siAVIL2, resulted in slower migration demonstrated by a wound-healing assay. FIG. 2E shows live cell tracking at an early time point of siAVIL1 transfection showed slowed cell migration compared with siCT. FIG. 2F shows mean velocities of all cells tracked in the experiment (n > 120 cells quantified per condition) (box, 25-75 percentile; whisker, 5-95 percentile; bar in the middle, median) (two-sided Student’s t-test). FIGS. 2G and 2H show RH30 cells infected with a viral vector expressing shAVIL1, which targets the same sequence as siAVIL, had reduced proliferation measured by cell counting (FIG. 2G) and MTT (FIG. 2H). FIG. 2I are subcutaneous xenografts of RH30 cells infected either with viruses expressing shAVIL1 or a control sequence shCT (n=10). All animals in the shCT group developed tumor, whereas the shAVIL1 group only had two small tumors. FIGS. 2J and 2K show comparison of tumor weight (FIG. 2J) and volume (FIG. 2K) between the two groups. FIG. 2L shows Kaplan-Meier survival curve for the shCT and shAVIL1 groups. All the shCT animals died or reached the endpoint for tumor size limitation, whereas none of the mice in the shAVIL1 group did (one mouse was euthanized as a control when the first shCT mice were terminated) (two-sided log-rank test). m-q RH30 cells stably expressing tet-inducible shAVIL or shCT were injected subcutaneously into nude mice. Animals were fed with doxycycline containing or control water. Tumors were harvested (m). Weights (n, p) and volumes (o, q) were measured. Data are presented as mean values ± SD in FIGS. 2C, 2G, 2H, 2J, 2K, and 2N-2Q. P value was calculated by standard two-tailed t-test. *p < 0.05, **p < 0.01, ***p < 0.001.

FIGS. 3A to 3E show AVIL is overexpressed in RMS. FIG. 3A shows FISH staining using AVIL probe on MSC, RH5, SMS-CTR, and RMS-13, showing that all the three RMS lines have more than four dots, whereas MSC cells only two. FIG. 3B shows qRT-PCR measuring AVIL level relative to GAPDH among RMS cell lines. FIG. 3C is a Western blot with AVIL antibody comparing AVIL protein expression in MSC and RMS cell lines. FIGS. 3D and 3E show qRT-PCR measuring AVIL level relative to that of GAPDH among PDX samples (FIG. 3D) and clinical RMS samples (FIG. 3E). Data are presented as mean values ± SD in FIGS. 3B, 3D, and 3E. P value was calculated by standard two-tailed t-test. *p < 0.05, **p < 0.01, ***p < 0.001.

FIGS. 4A to 4L show AVIL overexpression is necessary for RMS tumorigenesis. FIG. 4A shows RD cells were transfected with two siRNA targeting AVIL (siAVIL1 and siAVIL2), or siGL2 as control (siCT). Western blot analysis demonstrated the knocking down of AVIL protein expression. FIGS. 4B to 4D show crystal violet staining of RD (FIG. 4B), SMS-CTR (FIG. 4C), and MSC (FIG. 4D) cell cultures transfected with the same set of siRNAs as in FIG. 4A. FIG. 4E shows live cell imaging tracking individual cells of RD cells was observed over 24 h period of time, starting 24 h after transection with either siCT (left) or siAVIL1 (right). Shown are representative images depicting the starting timepoint of the experiment with overlaid lines tracking the movement of individual cells. FIG. 4F shows mean velocities of all cells tracked in the experiment depicted in FIG. 4E (n > 150 cells quantified per condition) (box, 25-75 percentile; whisker, 5-95 percentile; bar in the middle, median) (two-sided Student’s t-test). FIG. 4G shows live cell imaging for MSC cells using the same setup as in FIG. 4E. FIG. 4H shows quantification of velocities of all cells tracked in FIG. 4G (n>1000), FIG. 4I Xenografts of RD cells expressing shAVIL1, or control (shCT). The tumors were harvested at the end of the experiment and pictured. FIGS. 4J and 4K show tumor volume (FIG. 4J) and weight (FIG. 4K) comparison between the two groups. FIG. 4L shows percent survival of the animals was plotted according to Kaplan-Meier analysis (two-sided log-rank test). Data are presented as mean values ± SD in FIGS. 4J and 4K. P value was calculated by standard two-tailed t-test. *p < 0.05, **p < 0.01, ***p < 0.001.

FIGS. 5A to 5I show AVIL is a bona fide oncogene in RMS. FIGS. 5A and 5B show overexpressing AVIL in MSC (FIG. 5A) and RH4 (FIG. 5B) cells resulted in a higher proliferation rate measured by MTT. FIGS. 5C and 5D show overexpressing AVIL in MSC (FIG. 5C) and RH4 (FIG. 5D) cells resulted in higher motility measured by wound healing. FIG. 5E show focus formation assay. Quantification of foci resulted from oncogene cooperation assays. NIH3T3 cells were transfected with RAS and PAX3-FOXO1 with or without AVIL overexpression. Two-sided Student’s t-test. Each group is compared to NIH3T3 control group unless noted by a line. n=5. FIG. 5F shows focus formation assay. MSC cells were transfected with AVIL-expressing (AVIL) or control empty plasmid (CT). The quantitative difference of the foci number between the two groups was plotted. n=5. FIG. 5G shows MSC cells expressing control plasmid (CT) or AVIL (AVIL) were injected subcutaneously into the flanks of immunodeficient mice. The same animals received CT on the left side and AVIL on the right. Representative images were shown. FIGS. 5H and 5I show comparison of tumor volume (FIG. 5H) and weight (FIG. 5I) between the two groups. FIG. 5J shows representative hematoxylin and eosin staining of the tumors harvested from the mice. Histology analysis revealed histologic features of neoplasms. Data are presented as mean values ± SD in FIGS. 5A-5F, 5H, and 5I. P value was calculated by standard two-tailed t-test. *p < 0.05, **p < 0.01, ***p < 0.001.

FIGS. 6A to 6E show AVIL regulates PAX3-FOXO1 and RAS pathways. FIG. 6A shows MSC cells expressing empty vector (CT), or AVIL were harvested. RNAs were extracted and sequenced at three different passages. Volcano plot of expression data rendered using the EnhancedVolcano library with log2FoldChange cutoff of abs|2| and padj < 0.01. Colors represent gene as non-significant (grey- “NS”), passing log2FoldChange cutoff only (green- “log2 FC”, padj cutoff only (light blue- “p-value”) or passing both log2FoldChange and padj cutoffs (red- “p-value and Log2 FC”). Targets of PAX3-FOXO1 and RAS are highlighted. FIG. 6B shows GSEA analyses demonstrate the enrichment of PAX-FOXO1 gene expression signature that defines ARMS and its prognosis, and a gene set found in mouse MSC cells expressing PAX-FOXO1 fusion. FIG. 6C shows gene set for RAS signaling was also enriched. FIG. 6D shows qPCR validation for targets of PAX3-FOXO1 (left), and RAS (right). The levels of various transcripts were normalized against internal control GAPDH, then further normalized to that in MSC/CT. FIG. 6E is a Western blot that measured the protein level changes of total and phosphorylated MEK1/2 and ERK1/2. Data are presented as mean values ± SD in FIGS. 6E and 6E. P value was calculated by standard two-tailed t-test. *p < 0.05, **p < 0.01, ***p < 0.001.

FIGS. 7A to 7E show AVIL is overexpressed in other sarcomas, and its expression correlates with worse clinical outcomes. FIG. 7A shows cBioPortal analysis on AVIL gene. FIG. 7B shows relative AVIL expression against GAPDH was measured by qRT-PCR and compared in RH30, A673, TTC-466, and TC32 (Ewing sarcoma), 402-91 (myxoid liposarcoma), JN-DSRCT (desmoplastic small round cell sarcoma), SUCCS-1 (clear cell sarcoma), A2243 (synovial sarcoma), and OsACL (osteosarcoma) cells. FIGS. 7C and 7D show GESA analysis showed enrichment of Ewing family tumor signature (FIG. 7C), and EWS-FLI triggered Ewing sarcoma progenitor signature (FIG. 7D). FIG. 7E shows clinical analysis using TCGA sarcoma dataset. A two-class model stratified by AVIL showed that the high AVIL group has a shorter overall survival. Data are presented as mean values ± SD in FIGS. 7B, and P value was calculated by standard the two-tailed t-test. P values in e was calculated with two-sided log-rank test.

FIG. 8 shows detection of MARS-AVIL fusion in RMS. MARS-AVIL was measured by qRT-PCR and normalized against that of GAPDH. Data are presented as mean values ± SD. P value was calculated by standard two-tailed t-test. *p < 0.05, **p < 0.01, ***p < 0.001.

FIGS. 9A and 9B show MARS-AVIL, but not AVIL, had a partial rescue of cell growth caused by siAVIL1. FIG. 9A shows effect of siRNAs targeting different parts of AVIL. siAVIL1 silenced both MARS-AVIL and wild- type AVIL, whereas siAVIL2 silenced only wild-type AVIL. MARS-AVIL and AVIL level was measured by qRT-PCR and normalized against that of GAPDH. FIG. 9B shows RH30 cells were transfected by siCT or siAVIL1, and further transfected with MARS-AVIL or AVIL expression vector. Microscopic images of the various groups were shown on the left, and cell counting on the right. Data are presented as mean values ± SD. P value was calculated by standard two-tailed t-test. *p < 0.05, **p < 0.01, ***p < 0.001.

FIGS. 10A to 10D show MARS overexpressed had no obvious effect on RD and RH18. FIGS. 10A and 10C show cell proliferation measured by MTT in RD (FIG. 10A) and RH18 (FIG. 10C) cells stably expressing MARS or control vector. FIGS. 10B and 10D show cell motility measured by wound healing in RD (FIG. 10B) and RH18 (FIG. 10D) cells stably expressing MARS or control vector. Data are presented as mean values ± SD in FIGS. 10A to 10D. P value was calculated by standard two-tailed t-test.

FIGS. 11A to 11C show RH30 cells stably express tet-inducible shAVIL. FIG. 11A show upon addition of doxycycline, GFP positive cells were detected in both shCT and shAVIL cells. FIGS. 11B and 11C show qRT-PCR measuring the level of MARS-AVIL (FIG. 11B) and AVIL (FIG. 11C) in the absence or presence of doxycycline.

FIGS. 12A and 12B show qRT-PCR measuring AVIL expression in RMS PDX models (FIG. 12A) and clinical; samples (FIG. 12B). PAX3-FOXO1 and PAX7-FOXO1 were measured by qRT-PCR, and normalized against that of GAPDH. RH30 is the positive control for PAX3-FOXO1, and RMZ-RC2 as the positive control for PAX7-FOXO1. FIG. 12A shows RMS PDX models include two PAX3-FOXO1 positives, one PAX7-FOXO1 positive, and two PAX3/7-FOXO1 negative cases. FIG. 12B shows RMS clinical samples include 18 PAX3-FOXO1 positives, three PAX7-FOXO1 positives, and eight PAX3/7-FOXO1 negative cases.

FIGS. 13A and 13B show live cell imaging tracking individual cells over 24 hours window, 24 hrs after transfection. SMS-CTR cells were transfected with siCT or siAVIL. FIG. 13A show mean velocities of all cells tracked in the experiment (n > 4000 cells quantified per condition) (box, 25-75 percentile; whisker, 5-95 percentile; bar in the middle, median) (two-sided Student’s t-test). FIG. 13B contains representative images depicting the starting timepoint of the experiment with overlaid lines tracking the movement of individual cells.

FIGS. 14A to 14D show MARS-AVIL transforms MSC. FIG. 14A shows a focus formation assay. MSC cells were transfected with MARS-AVIL-expressing (MARS-AVIL) or control empty plasmid (CT). The quantitative difference of the foci number between the two groups was plotted. n=5. FIG. 14B shows MSC cells expressing control plasmid (CT) or MARS-AVIL were injected subcutaneously into the flanks of immunodeficient mice. The same animals received CT on the left side, and MARS-AVIL on the right. Representative images were shown. n=7. FIGS. 14C and 14D show comparison of tumor volume (FIG. 13C) and weight (FIG. 14D) between the two groups. FIG. 14E shows representative hematoxylin and eosin staining of the tumors harvested from the mice. Histology analysis revealed histologic features of neoplasms. Data are presented as mean values ± SD in FIGS. 14A, 14C, and 14D. P value was calculated by standard two- tailed t-test. *p < 0.05, **p < 0.01, ***p < 0.001.

FIGS. 15A and 15B show RNA-Seq analysis on MSC overexpressing AVIL compared with MSC control. FIG. 15A shows principle component analysis (PCA) plot. PCA plot is produced from shrunken log2FoldChange values obtained from Deseq2 using Approximate Posterior Estimation for GLM (apeglm) shrinkage estimation. Each dot represents a sample replicate. PC1 separation characterizes the trend exhibited by expression profiles between the two groups. FIG. 15B shows GSEA analysis. Genes bound by PAX3-FOXO1 from ChlP experiment were enriched.

FIGS. 16A and 16C show RNA-Seq analysis on MSC cells overexpressing MARS-AVIL or AVIL. Upper part is the Venn diagram showing differential expressed genes in MARS-AVIL (FIG. 16A) and AVIL (FIG. 16B) overexpressed MSC cells. Lower part is the GO term analysis of MARS-AVIL uniquely upregulated 217 genes (FIG. 16C).

FIGS. 17A to 17D show Rhabdomyosarcoma RH30 cells infected with viral shRNA constructs, and stable cells selected by antibiotics. Cell number was counted on day 0 and day4 (FIG. 17A). MTT was conducted on a sperate experiment. OD at 570 nm was measured (FIG. 17B). These stable cells were injected into nude mice subcutaneously. Tumors were harvested. All 10 injections with RH30 cells infected with shCT developed tumors. Only ⅒ in the group of RH30 cells infected with shAVIL593 developed two small tumors. Tumor weight and volume were plotted in FIG. 17C. FIG. 17D is the Kaplan-Meier curve of the animal survival.

FIGS. 18A to 18C show Rhabdomyosarcoma RD cells transfected with control siRNA, or two siRNAs against AVIL. The expression of AVIL and LIN28B (AVIL downstream target) were measured by qRT-PCR (FIG. 18A). Stable cells of RD infected with control or shRNA targeting AVIL were established. These cells were injected into nude mice subcutaneously. Tumors were harvested. Tumor weight and volume were plotted in FIG. 18B. FIG. 18C is the Kaplan-Meier curve of the animal survival.

FIGS. 19A to 19D show GBM cells U251 were transfected with control siRNA, or two siRNAs against AVIL. Western blot was used to measure AVIL protein level (FIG. 19A). Crystal violet staining was used to show the effect of the siRNAs on cell growth (FIG. 19B). Wound healing assay was used to measure cell migration (FIG. 19C). Matrigel coated transwell assay was used to measure invasion (FIG. 19D).

FIGS. 20A to 20C show GBM cells A172 transfected with control siRNA, or two siRNAs against AVIL. Crystal violet staining was used to show the effect of the siRNAs on cell growth (FIG. 20A). Wound healing assay was used to measure cell migration (FIG. 20B). Matrigel coated transwell assay was used to measure invasion (FIG. 20C).

FIGS. 21A and 21B show in vivo effect of silencing AVIL. Stable cells of U251 infected with control or shRNA targeting AVIL were established. Representative MRI images are shown in FIG. 21A. Tumor volume were plotted in FIG. 21B. FIG. 21C is the Kaplan-Meier curve of the animal survival.

FIGS. 22A to 22C show Glioblastoma stem cells GSC11 and GSC627 were transfected with sicontrol or two different siRNAs targeting AVIL. qRT-PCR measured the knockdown of AVIL expression in GSC11 (FIG. 22A). GSC cell proliferation was measured by MTT for GSC11 (FIG. 22B), and GSC627 (FIG. 22C).

FIGS. 23A to 23D show GBM PDX short cultures, GBM10, GBM76 and GBM108 obtained from Mayo Clinic. They were transfected with sicontrol or three different siRNAs targeting AVIL. qRT-PCR measured the knockdown of AVIL expression (FIGS. 23A-23C). GBM10 cell proliferation was measured by MTT (FIG. 23D).

FIGS. 24A and 24B show syngeneic mouse line CT2A cells transfected with sicontorl or two siRNA targeting AVIL. FIG. 24A is phase picture. FIG. 24B quantifies cell number in each group.

FIG. 25A shows the expression of AVIL mRNA in MGC-803, SGC-7901, AGS and GES-1 cells measured by qRT-PCR, and normalized to GAPDH. The ratio was further normalized to that in GES-1. FIG. 25B shows the expression of AVIL protein measured by Western blot. The ratio of AVIL to beta-actin was labeled. FIG. 25C shows two separate siRNAs targeting AVIL gene transfected into AGS and MGC-803 cells. Relative expression of AVIL normalized against GAPDH was measured by qRT-PCR. The level in the control siRNA (siNC) group was set as “1”. FIG. 25D shows AVIL protein expression in siRNA transfected samples analyzed by Western blot. The ratio of AVIL to beta-actin was labeled. FIG. 25E shows CCK-8 used to evaluate cell proliferation in the siRNA transfected AGS, and MGC-803 cells. FIG. 25F shows flow cytometry analysis showing apoptosis levels in siRNA transfected cells. The apoptotic cell percentage (%) is plotted in a histogram. FIG. 25G shows a wound healing assay used to measure cell migration. FIG. 25H shows cell invasion ability measured by matrigel coated transwell assay.

FIG. 26A shows the expression levels of AVIL mRNA measured by qRT-PCR in SGC-7901 cells stably expressing one of the three lentiviral vectors: shAVIL1 (350), shAVIL2 (351), or the negative control shCT (96). FIG. 26B shows a CCK-8 assay used to measure cell proliferation in these stable cells. FIG. 26C shows a wound healing assay measuring cell migration. FIG. 26D shows quantification of the cell migration in a histogram. FIG. 26E shows harvested xenografts from mice injected with SGC-7901-shCT (96) and SGC-7901-shAVIL2 (351) cells. FIG. 26F shows tumor weight comparison between the two animal groups. FIG. 26G shows tumor volume comparison between the two animal groups. FIG. 26H shows a Tet-on shRNA system. AVIL RNA level was measured by qRT-PCR in the stable cells in the presence of absence of doxycycline. FIG. 26I shows DOX-dependent xenografts harvested from shCT (564) versus shAVIL (565).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y″’, where ‘x’ and ‘y’ are numerical values, includes “about ‘x’ to about ‘y″’.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/- 10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “advillin” and “AVIL” can be used interchangeably, and refer to a protein encoded by a gene in humans with a cytogenetic location of 12q14.1 and a molecular location of base pairs 57,797,376 to 57,818,704 on chromosome 12 (Homo sapiens Annotation Release 109, GRCh38.p12). The protein encoded by this gene is a member of the gelsolin/villin family of actin-regulatory proteins. AVIL has also been referred to as Actin-binding protein DOC6, DOC6, P92, ADVIL.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians’ Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, Tr-Tr interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof. Thus, the subject can be a human, non-human primate, horse, pig, rabbit, dog, sheep, goat, cow, cat, guinea pig or a rodent. The term does not denote a particular age or sex. Moreover, adult and newborn subjects, as well as fetuses, whether male or female, are intended to be included. A “patient” refers to a subject afflicted with a clinical condition, disease or disorder.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer, or a disease or disorder associated with increased, aberrant, or dysfunctional levels of AVIL. The effect can be therapeutic in terms of a partial or complete cure of a disease, condition, symptom or adverse effect attributed to the disease, disorder, or condition. The term “treatment” as used herein can include any treatment of cancer, or a disease or disorder associated with increased, aberrant, or dysfunctional levels of AVIL, in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay. For example, an IC₅₀ for the activity of a compound disclosed herein can be determined in an in vitro or cell-based assay system using the methods described herein. Frequently, an assay, including suitable assays for AVIL, can make use of a suitable cell-line, e.g. a cell line that either expresses endogenously a target of interest, or has been transfected with a suitable expression vector that directs expression of a recombinant form of the target such as AVIL. For example, the IC₅₀ for the compounds disclosed herein can be determined using appropriate cells lines, e.g., glioblastoma cells (U87) and astrocyte cells (non-cancer control).

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e. one atmosphere).

In various aspects, the present disclosure pertains to certain compounds and methods that are useful to regulate, limit, or inhibit the expression of AVIL (advillin) in tissue of a mammal, particularly where there are increased, aberrant, or dysfunctional levels of AVIL. It has been found that AVIL expression, or over-expression, is associated with the genesis and growth of certain forms of cancerous tumors. For example, it has been found that AVIL is overexpressed in the vast majority, if not all of human glioblastomas (GBMs). It has been found that GBM cells depend on the overexpression of AVIL for increased survival and migration. Silencing AVIL induced GBM cell death in vitro, and prevented GBM xenograft formation and growth in animal models. Silencing AVIL also dramatically changed cell morphology, and reduced cell migration/invasion ability. In contrast, normal astrocytes express very low levels of AVIL, and silencing AVIL had no obvious effect on cell growth or morphology of normal astrocytes. Clinically, higher expression of AVIL has been correlated with worse patient outcome in GBMs as well as in lower-grade gliomas. In addition to gliomas, lung cancer, bladder cancer, and renal cancer patients with high level of AVIL expression also had worse prognosis. Therefore, in various aspects of the present disclosure, certain compounds are provided, and methods useful for targeting AVIL as an oncogene and a therapeutic target. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Silencing Oligonucleotides

Disclosed herein are gene silencing oligonucleotides (e.g. siRNA and shRNA) that are capable of silencing AVIL or the MARS-AVIL fusion.

Examples of gene silencing oligonucleotides include antisense molecules, shRNA, miRNA, siRNA, and oligonucleotides used with gene editing techniques, such as CRISPR or TALON.

In some cases, the silencing oligonucleotide is an siRNA. Therefore, in various aspects the silencing oligonucleotide is an siRNA comprising the nucleic acid sequence
5′-GCUUCUGGCAAAGGAUAUU-3′ (siAVIL 593, SEQ ID NO:116),
5′-CCACACAGCUGGACGACUA-3′ (siAVIL265, SEQ ID NO:117),
5′-GCAAAGGAUAUUCGAGACA-3′ (siAVIL600, SEQ ID NO:118),
5′-GCACCAAUGUGGAGACCGU-3′ (siAVIL1000, SEQ ID NO:119),
5′-GGAAAGAAUGGUCGAUGAU-3′ (siAVIL1187, SEQ ID NO:120),
5′-GCAUUCCUUGCUUGUUAUA-3′ (siAVIL2651, SEQ ID NO:121), or
5′-GAGCUUAAGAAAAUGGAGC-3′ (siMARS-AVIL, SEQ ID NO:122). In some embodiments, the silencing oligonucleotide has a nucleic acid sequence complementary to the nucleic acid sequence disclosed herein.

Thus, in various aspects, the gene silencing oligonucleotide targets a region with the nucleic acid sequence:

ATGAGACTGTTCGTGAGTGATGGCGTCCCGGGTTGCTTGCCGGTGCTGGC
CGCCGCCGGGAGAGCCCGGGGCAGAGCAGAGGTGCTCATCAGCACTGTAG
GCCCGGAAGATTGTGTGGTCCCGTTCCTGACCCGGCCTAAGGTCCCTGTC
TTGCAGCTGGATAGCGGCAACTACCTCTTCTCCACTAGTGCAATCTGCCG
ATATTTTTTTTTGTTATCTGGCTGGGAGCAAGATGACCTCACTAACCAGT
GGCTGGAATGGGAAGCGACAGAGCTGCAGCCAGCTTTGTCTGCTGCCCTG
TACTATTTAGTGGTCCAAGGCAAGAAGGGGGAAGATGTTCTTGGTTCAGT
GCGGAGAGCCCTGACTCACATTGACCACAGCTTGAGTCGTCAGAACTGTC
CTTTCCTGGCTGGGGAGACAGAATCTCTAGCCGACATTGTTTTGTGGGGA
GCCCTATACCCATTACTGCAAGATCCCGCCTACCTCCCTGAGGAGCTGAG
TGCCCTGCACAGCTGGTTCCAGACACTGAGTACCCAGGAACCATGTCAGC
GAGCTGCAGAGACTGTACTGAAACAGCAAGGTGTCCTGGCTCTCCGGCCT
TACCTCCAAAAGCAGCCCCAGCCCAGCCCCGCTGAGGGAAGGGCTGTCAC
CAATGAGCCTGAGGAGGAGGAGCTGGCTACCCTATCTGAGGAGGAGATTG
CTATGGCTGTTACTGCTTGGGAGAAGGGCCTAGAAAGTTTGCCCCCGCTG
CGGCCCCAGCAGAATCCAGTGTTGCCTGTGGCTGGAGAAAGGAATGTGCT
CATCACCAGTGCCCTCCCTTACGTCAACAATGTCCCCCACCTTGGGAACA
TCATTGGTTGTGTGCTCAGTGCCGATGTCTTTGCCAGGTACTCTCGCCTC
CGCCAGTGGAACACCCTCTATCTGTGTGGGACAGATGAGTATGGTACAGC
AACAGAGACCAAGGCTCTGGAGGAGGGACTAACCCCCCAGGAGATCTGCG
ACAAGTACCACATCATCCATGCTGACATCTACCGCTGGTTTAACATTTCG
TTTGATATTTTTGGTCGCACCACCACTCCACAGCAGACCAAAATCACCCA
GGACATTTTCCAGCAGTTGCTGAAACGAGGTTTTGTGCTGCAAGATACTG
TGGAGCAACTGCGATGTGAGCACTGTGCTCGCTTCCTGGCTGACCGCTTC
GTGGAGGGCGTGTGTCCCTTCTGTGGCTATGAGGAGGCTCGGGGTGACCA
GTGTGACAAGTGTGGCAAGCTCATCAATGCTGTCGAGCTTAAGAAAATGG
AGCTGGCGCTGGTGCCTGTGAGCGCCCACGGCAACTTCTATGAGGGGGAC
TGCTACGTCATCCTCTCGACCCGGAGAGTGGCCAGTCTCCTATCCCAGGA
CATCCACTTCTGGATCGGGAAGGACTCCTCCCAGGATGAGCAAAGCTGCG
CAGCCATATATACCACACAGCTGGACGACTACCTGGGAGGCAGCCCTGTG
CAGCACCGAGAGGTCCAGTACCATGAGTCAGACACTTTCCGTGGCTACTT
CAAGCAGGGCATCATCTACAAGCAGGGGGGTGTCGCCTCTGGGATGAAGC
ACGTGGAGACCAATACCTACGACGTGAAGCGGCTGCTACATGTGAAAGGG
AAAAGAAACATCAGGGCTACCGAGGTGGAAATGAGCTGGGACAGTTTCAA
CCGAGGTGATGTCTTCTTGCTGGACCTTGGGAAAGTCATCATCCAATGGA
ATGGCCCAGAGAGCAACAGTGGGGAGCGCCTGAAGGCTATGCTTCTGGCA
AAGGATATTCGAGACAGGGAGCGAGGGGGCCGTGCTAAAATAGGAGTGAT
CGAGGGAGACAAGGAGGCAGCCAGCCCAGAGCTGATGAAGGTCCTTCAGG
ACACCCTTGGCCGACGCTCCATTATCAAGCCTACAGTCCCTGATGAGATC
ATAGATCAGAAGCAGAAATCAACTATCATGTTGTATCATATCTCAGATTC
AGCTGGGCAGCTGGCAGTCACAGAGGTAGCAACAAGGCCTCTGGTCCAGG
ACTTACTGAACCATGATGACTGCTACATCCTGGACCAAAGTGGAACCAAA
ATCTACGTGTGGAAAGGAAAAGGAGCCACAAAGGCTGAAAAACAGGCAGC
CATGTCTAAAGCGCTGGGCTTCATCAAGATGAAGAGCTACCCCAGCAGCA
CCAATGTGGAGACCGTCAACGATGGTGCTGAGTCGGCCATGTTCAAGCAG
CTGTTCCAGAAGTGGTCAGTAAAGGACCAGACCATGGGCCTGGGGAAAAC
GTTCAGCATTGGTAAAATTGCTAAAGTTTTCCAGGATAAATTTGATGTGA
CTCTGCTACACACCAAGCCAGAGGTAGCTGCCCAGGAAAGAATGGTCGAT
GATGGCAACGGAAAAGTTGAGGTCTGGAGAATTGAGAACCTGGAGCTGGT
CCCTGTGGAGTATCAATGGTATGGCTTCTTTTATGGGGGAGACTGTTATC
TGGTCCTCTACACATACGAGGTAAATGGGAAGCCACATCACATCTTGTAC
ATCTGGCAGGGCCGCCACGCCTCACAGGATGAGCTGGCAGCCTCAGCATA
CCAGGCAGTGGAGGTGGATCGGCAGTTTGATGGGGCTGCTGTGCAGGTTC
GAGTCAGGATGGGAACGGAGCCACGCCACTTCATGGCCATCTTCAAAGGG
AAGCTAGTTATCTTTGAGGGTGGGACTTCCAGGAAGGGAAATGCCGAGCC
TGACCCTCCAGTAAGACTCTTCCAAATTCATGGAAATGACAAATCTAACA
CCAAAGCAGTGGAAGTTCCAGCCTTTGCCTCCTCCCTAAACTCCAATGAT
GTCTTTCTGCTGCGAACTCAGGCAGAGCACTACCTGTGGTATGGCAAGGG
GTCTAGTGGGGATGAGCGGGCAATGGCTAAGGAGCTGGCCAGCCTTCTCT
GTGATGGCAGCGAGAACACTGTGGCCGAGGGCCAGGAGCCAGCCGAGTTC
TGGGACCTACTGGGAGGGAAAACTCCCTATGCCAATGATAAAAGACTTCA
GCAGGAAATCCTAGATGTCCAGTCTCGTCTCTTTGAATGTTCCAATAAGA
CCGGCCAATTCGTTGTCACTGAGATCACAGACTTCACCCAGGATGACCTG
AACCCTACTGACGTGATGCTCCTAGATACCTGGGACCAGGTGTTCTTGTG
GATTGGGGCTGAGGCCAATGCCACGGAGAAGGAGAGTGCCCTTGCCACAG
CACAGCAGTACCTGCACACTCACCCCAGCGGCCGAGATCCCGACACACCA
ATCCTGATCATTAAGCAGGGGTTTGAGCCTCCCATCTTCACAGGCTGGTT
CCTAGCCTGGGACCCTAACATTTGGAGTGCAGGAAAAACATATGAACAAT
TAAAAGAAGAGCTGGGAGATGCTGCTGCTATCATGCGAATCACTGCTGAC
ATGAAGAATGCAACCCTCTCCCTGAATTCTAATGACAGTGAGCCAAAATA
TTACCCTATAGCAGTTCTGTTGAAAAACCAGAATCAGGAGCTGCCTGAGG
ATGTAAACCCTGCCAAAAAGGAGAATTACCTCTCTGAACAGGACTTTGTG
TCTGTGTTTGGCATCACAAGAGGGCAATTTGCAGCTCTGCCTGGCTGGAA
ACAGCTCCAAATGAAGAAAGAAAAGGGGCTTTTCTAA (MARS-AVIL,
SEQ ID NO:8).

Thus, in various aspects, the gene silencing oligonucleotide targets a region with the nucleic acid sequence:

ATGCCTCTGACCAGTGCCTTCAGGGCTGTGGACAACGACCCTGGGATCAT
TGTCTGGAGAATAGAGAAAATGGAGCTGGCGCTGGTGCCTGTGAGCGCCC
ACGGCAACTTCTATGAGGGGGACTGCTACGTCATCCTCTCGACCCGGAGA
GTGGCCAGTCTCCTATCCCAGGACATCCACTTCTGGATCGGGAAGGACTC
CTCCCAGGATGAGCAAAGCTGCGCAGCCATATATACCACACAGCTGGACG
ACTACCTGGGAGGCAGCCCTGTGCAGCACCGAGAGGTCCAGTACCATGAG
TCAGACACTTTCCGTGGCTACTTCAAGCAGGGCATCATCTACAAGCAGGG
GGGTGTCGCCTCTGGGATGAAGCACGTGGAGACCAATACCTACGACGTGA
AGCGGCTGCTACATGTGAAAGGGAAAAGAAACATCAGGGCTACCGAGGTG
GAAATGAGCTGGGACAGTTTCAACCGAGGTGATGTCTTCTTGCTGGACCT
TGGGAAAGTCATCATCCAATGGAATGGCCCAGAGAGCAACAGTGGGGAGC
GCCTGAAGGCTATGCTTCTGGCAAAGGATATTCGAGACAGGGAGCGAGGG
GGCCGTGCTAAAATAGGAGTGATCGAGGGAGACAAGGAGGCAGCCAGCCC
AGAGCTGATGAAGGTCCTTCAGGACACCCTTGGCCGACGCTCCATTATCA
AGCCTACAGTCCCTGATGAGATCATAGATCAGAAGCAGAAATCAACTATC
ATGTTGTATCATATCTCAGATTCAGCTGGGCAGCTGGCAGTCACAGAGGT
AGCAACAAGGCCTCTGGTCCAGGACTTACTGAACCATGATGACTGCTACA
TCCTGGACCAAAGTGGAACCAAAATCTACGTGTGGAAAGGAAAAGGAGCC
ACAAAGGCTGAAAAACAGGCAGCCATGTCTAAAGCGCTGGGCTTCATCAA
GATGAAGAGCTACCCCAGCAGCACCAATGTGGAGACCGTCAACGATGGTG
CTGAGTCGGCCATGTTCAAGCAGCTGTTCCAGAAGTGGTCAGTAAAGGAC
CAGACCATGGGCCTGGGGAAAACGTTCAGCATTGGTAAAATTGCTAAAGT
TTTCCAGGATAAATTTGATGTGACTCTGCTACACACCAAGCCAGAGGTAG
CTGCCCAGGAAAGAATGGTCGATGATGGCAACGGAAAAGTTGAGGTCTGG
AGAATTGAGAACCTGGAGCTGGTCCCTGTGGAGTATCAATGGTATGGCTT
CTTTTATGGGGGAGACTGTTATCTGGTCCTCTACACATACGAGGTAAATG
GGAAGCCACATCACATCTTGTACATCTGGCAGGGCCGCCACGCCTCACAG
GATGAGCTGGCAGCCTCAGCATACCAGGCAGTGGAGGTGGATCGGCAGTT
TGATGGGGCTGCTGTGCAGGTTCGAGTCAGGATGGGAACGGAGCCACGCC
ACTTCATGGCCATCTTCAAAGGGAAGCTAGTTATCTTTGAGGGTGGGACT
TCCAGGAAGGGAAATGCCGAGCCTGACCCTCCAGTAAGACTCTTCCAAAT
TCATGGAAATGACAAATCTAACACCAAAGCAGTGGAAGTTCCAGCCTTTG
CCTCCTCCCTAAACTCCAATGATGTCTTTCTGCTGCGAACTCAGGCAGAG
CACTACCTGTGGTATGGCAAGGGGTCTAGTGGGGATGAGCGGGCAATGGC
TAAGGAGCTGGCCAGCCTTCTCTGTGATGGCAGCGAGAACACTGTGGCCG
AGGGCCAGGAGCCAGCCGAGTTCTGGGACCTACTGGGAGGGAAAACTCCC
TATGCCAATGATAAAAGACTTCAGCAGGAAATCCTAGATGTCCAGTCTCG
TCTCTTTGAATGTTCCAATAAGACCGGCCAATTCGTTGTCACTGAGATCA
CAGACTTCACCCAGGATGACCTGAACCCTACTGACGTGATGCTCCTAGAT
ACCTGGGACCAGGTGTTCTTGTGGATTGGGGCTGAGGCCAATGCCACGGA
GAAGGAGAGTGCCCTTGCCACAGCACAGCAGTACCTGCACACTCACCCCA
GCGGCCGAGATCCCGACACACCAATCCTGATCATTAAGCAGGGGTTTGAG
CCTCCCATCTTCACAGGCTGGTTCCTAGCCTGGGACCCTAACATTTGGAG
TGCAGGAAAAACATATGAACAATTAAAAGAAGAGCTGGGAGATGCTGCTG
CTATCATGCGAATCACTGCTGACATGAAGAATGCAACCCTCTCCCTGAAT
TCTAATGACAGTGAGCCAAAATATTACCCTATAGCAGTTCTGTTGAAAAA
CCAGAATCAGGAGCTGCCTGAGGATGTAAACCCTGCCAAAAAGGAGAATT
ACCTCTCTGAACAGGACTTTGTGTCTGTGTTTGGCATCACAAGAGGGCAA
TTTGCAGCTCTGCCTGGCTGGAAACAGCTCCAAATGAAGAAAGAAAAGGG
GCTTTTCTAA (AVIL, SEQ ID NO:9).

Thus, in various aspects, the gene silencing oligonucleotide targets a region with the nucleic acid sequence:

CCCTTCTGTGGCTATGAGGAGGCTCGGGGTGACCAGTGTGACAAGTGTGG
CAAGCTCATCAATGCTGTCGAGCTTAAGGTAAGAGGAGGGTCTCCATGGG
AGCCCGGAAGGAGACAGTCCTTATTCTTAAGGGACGCCCTTCCTGTCCCA
TTTAGGATTTTTATTTTAGCATTCTAGACCTTTAACCAGTGGCCCTTTCT
GCCCCATCTCAGCAACTCGTCATTTAGTTCATTTGAAAAATACTCCCTGA
GCACTTACTATGTGCTGAGCACTGTGCTAAGAAACAGACAAGTTGAGTTT
ATATTTCCCAACCTTTGGTCTTGCATCACACCACCTGATCTACATTTGTT
TAGATCCTTCTCTCTTCTTTAAATATCTTCTCTTCTAGCCATTTGTTAAT
TTATTTCTTCATTCATTTATTCAACATACATTTTTTGAATCCTTAGAATA
GGCCAATTACAATTCTGCGTGCTGGGGGTATGGCAATGAATAAGACAGAT
TTTATTTAGTATGGTCAGGAGACCAACACTAACTAAGGAAATGATATAGA
ATAAACTGCCAGGAAAATGTGACAGAGAGTGACTTCTGGGGGAAGATCTA
ACTTAGGTAGATAGGGAAGGCCTTTCAGAGGCAGTAACATTTGAACTGAG
AGTTTAACCAAAGAATTAGGAGCAAGCCAGGCAATAAGAGGTTAAGAATG
TTCTAAGCGGAAGGACTAGGAAATGCAAAGGCCATGAGGCAGCTAAGAGC
TGAAAAGGCAGAATTGGCAAGGCCAGTATAGCACTAGCACAGTGAGTGAG
GAGGAGGGTAGACTGAGATGAGGTGAGAGAGATGGCAGGGGCCAGATCAC
CAGGGCCTGTGAAGCCATTGTGGGAATTTAGATTTTATTCTGGTGAATGA
CAAGCTGCTCTGTGGAAAATGGATGGCAGGGAAGCAAGAAATAAAGTATG
TTGAAGGGGCTGGGCATTGGTGGCTCATACCTATAATCCCAGCACTTTGG
GAGGCCAAGCTGGGTGGATCACTTGAAGCCACGAGTTTGAGACCAGCCTG
GCCTGTAACATGGCAACCCTGTCTCTACTGAAAATACAGAAATGGCTAGG
TGCGGTGGCTCACGCCTGTAATCCCAGCACTTTGGGAGGCTGAGGTGGGC
AGATCACTTGAGGTCAGGAGTTTGAGACCAGCCTGGCCAACATGGTGAAA
CCCTGTCTCTACTAAAAAAATACAAAAATTAGCTGGGTGTGGTGGCGGAC
CCCTGTAATCCCCGCTTCTTGGGAGGCTGAAGCTTGAGAATCACTTGAAC
CTGGGAGGCAGAGGTTGCAGTGAGCTGAGATTGCGCCACTGCATTCCAGC
CTGGGTTACAAAGTAAGACTCTGTCTAAAAAAAAAAAAAAACCACAAAAA
TTATCCAGGCCGTGGTGGCGCACACCTGTAATCCCAGCTACTTGGGAGGC
TGAGGCAGAAGAATTTCTTGAACCTGGGAAGCGGAGGTTGCAGTGAGCCA
AGATTGCCACTGCATTCCAGCCTGGGTGACAGAGCAAGACTGTTCCCCCT
CCACCAAAAAAAAAAGTATGGAGAAGGATTTGGAGGCAACAAGGCTACAA
CCCAGAAATGGGATAATGGTGGCTTGCACTAGGTTAGTAGCACTAGAAAT
GGAAAAGAAAAAATAGACTTGAGGTATATTTTTGGAGGTAAACTCCACGG
AGTTGGATAGACTGGAATTTGGGATGGTCAGGAAAAAGATGCCTTGTAAG
TTTTTGGCCTGAGTAGATGGTAGTACCATCTGTGAACGTGAGTGAGGAGA
ACAGGCTTTCAGGTGGGATGCAGCCATACTGTTTGGGTTTGTTAAGCTTG
AGATGCCTGTTAGATAGCCAGGCAGAGAAGTCAGGTAGACAGTTGGATAT
TAGGCATATGGAGTTCAGGAGAGAGGTCCTGAGGAATTATAATTTCTCAC
CTTCAAAAACTCAGATCAAAACTCACTTGGGGCCAGGTGTGGTGGCTCAC
GCTTGTAATCCCAGGACTTTGGGAAGCTGAGGGACCAAGGTTCAGATCCG
GGGTGATGCAAGCAGAGCTCACAGAAGCCAGAGGGATGGCCACGTTAACC
CTGTGGCTGGCAGCCTCACATCCTGTAAGGTCATTGAGAACATCTCCCCT
CCTTCTCCCCACCACCTGCACCGAATGACAGAGAGGAGAGGGAGGCTCAT
GGGGAAGGGAGGTGGGAGGGGATGTAGCCTATACCCACCCCTTGTCCTTC
CAGAAAATGGAGCTGGCGCTGGTGCCTGTGAGCGCCCACGGCAACTTCTA
TGAGGGGGACTGCTACGTCATCCTCTCGGTGAGCACCCTCCTCCCCACTC
CTGTGAGCCTCTCCCCTGGGGCCAGTTCTTGGGCTGTACTGGGAGATGTG
CTGGTGAGGGAAGGTAGTCAGTCCCTGGTATCGGTCTCAATGGTTCCTCA
GTGCTATGCAGAGCAGACCTCCAGTCTGACTATCAAAGGCCATCATTAAG
CCTGGGTTTTTAATGCTGCATAAGAGGATGGGGGTAGGACCCTCAGTCTG
AACCTCAGTGAGCCACCCACAAGCAGCACTTCTCCCGGGCCTAGGCCCCA
CATTAAAGTGGTGTGGAAGAAGGCCTGATAAATGCAAGGCCTGTCTCTAG
CCTCAAAGTGCTATTCTCATGGAGGAGACTCACATGAACGGTGCTGACAG
GGTCGTGCAGGTGTACAGATGAGCACTGTGCCAGCTGTGTGAGTGCAAGG
GACCAAGGGGACCTGTGAGGTAAGCCTGCAACCCGGCTGGGAAAAGATCC
CCGTTGGTATCTATTTATTTGGGGAACAACAAGCTGTCAACTTTCCCCAA
ATGCGGGGAGGAATTTGGGCTGCCAAAGTTTATGCCATGATCGCAGGTCT
GCTCGGGGGCCAGGGCAGCTCCCACATCCACTGCAGGAGAGAGGGCCCTC
AGTCTGCCAAGGGCATGCACATGTGCTGCATTCTCCCCATAACAGGGGCC
AGCAGCAAAGGGGAGGTGAGCTGGCCTCTGACCTGATCTCTCTGACTACC
TCCCAGACCCGGAGAGTGGCCAGTCTCCTATCCCAGGACATCCA (MARS
-AVIL DNA, SEQ ID NO:10).

Thus, in various aspects, the gene silencing oligonucleotide targets a region with the nucleic acid sequence:

CCCTTCTGTGGCTATGAGGAGGCTCGGGGTGACCAGTGTGACAAGTGTGG
CAAGCTCATCAATGCTGTCGAGCTTAAGAAAATGGAGCTGGCGCTGGTGC
CTGTGAGCGCCCACGGCAACTTCTATGAGGGGGACTGCTACGTCATCCTC
TCGACCCGGAGAGTGGCCAGTCTCCTATCCCAGGACATCCA (MARS-AV
IL RNA, SEQ ID NO:11).

In some embodiments, the siRNA or shRNA target the nucleic acid sequence:

5′-GCTTCTGGCAAAGGATATT-3′ (SEQ ID NO:1), 5′-CTTCAG
GGCTGTGGACAAC-3′(SEQ ID NO:2), 5′-CCACACAGCTGGACGA
CTA-3′ (SEQ ID NO:3),5′-TCCGTGGCTACTTCAAGCA-3′ (SE
Q ID NO:4), 5′-AAGGCTATGCTTCTGGCAA-3′(SEQ ID NO:5)
, 5′-GCACCAATGTGGAGACCGT-3′ (SEQ ID NO:6), or5′-TA
TAACAAGCAAGGAATGC-3′ (SEQ ID NO:7).

In some cases, the silencing oligonucleotide is an siRNA. Therefore, in various aspects the silencing oligonucleotide is an siRNA comprising the nucleic acid sequence:

5′-CUGGAGAAUAGAGAAAAUGGAGC-3′ (siAVIL 179, SEQ ID
NO:12),

5′-UACAUGUGAAAGGGAAAAGAAAC-3′ (siAVIL535, SEQ ID N
O:13),

5′-UAGAUCAGAAGCAGAAAUCAACU-3′ (siAVIL850, SEQ ID N
O:14),

5′-AGCAGAAAUCAACUAUCAUGUU-3′ (siAVIL859, SEQ ID NO
:15),

5′-AUCAACUAUCAUGUUGUAUCAUA-3′ (siAVIL866, SEQ ID N
O:16),

5′-AACUAUCAUGUUGUAUCAUAUCU-3′ (siAVIL869, SEQ ID N
O:17),

5′-AUCAUGUUGUAUCAUAUCUCAGA-3′ (siAVIL873, SEQ ID N
O:18),

5′-UUGUAUCAUAUCUCAGAUUCAGC-3′ (siAVIL879, SEQ ID N
O:19),

5′-AGCCACAAAGGCUGAAAAACAGG-3′ (siAVIL1022, SEQ ID
NO:20),

5′-AGCAUUGGUAAAAUUGCUAAAGU-3′ (siAVIL1203, SEQ ID
NO:21),

5′-UUGGUAAAAUUGCUAAAGUUUUC-3′ (siAVIL1207, SEQ ID
NO:22),

5′-UGGUAAAAUUGCUAAAGUUUUCC-3′ (siAVIL1208, SEQ ID
NO:23),

5′-AUCAAUGGUAUGGCUUCUUUUAU-3′ (siAVIL1360, SEQ ID
NO:24),

5′-CUCUACACAUACGAGGUAAAUGG-3′ (siAVIL1404, SEQ ID
NO:25),

5′-GCCACUUCAUGGCCAUCUUCAAA-3′ (siAVIL1573, SEQ ID
NO:26),

5′-UCCAGUAAGACUCUUCCAAAUUC-3′ (siAVIL1655, SEQ ID
NO:27),

5′-CUCUUCCAAAUUCAUGGAAAUGA-3′ (siAVIL1665, SEQ ID
NO:28),

5′-UCCAAAUUCAUGGAAAUGACAA-3′ (siAVIL1669, SEQ ID N
O:29),

5′-UUCAUGGAAAUGACAAAUCUAAC-3′ (siAVIL1675, SEQ ID
NO:30),

5′-AUGGAAAUGACAAAUCUAACACC-3′ (siAVIL1678, SEQ ID
NO:31),

5′-CUCCCUAAACUCCAAUGAUGUCU-3′ (siAVIL1730, SEQ ID
NO:32),

5′-GUCUCUUUGAAUGUUCCAAUAAG-3′ (siAVIL1975, SEQ ID
NO:33),

5′-UGCAGGAAAAACAUAUGAACAAU-3′ (siAVIL2276, SEQ ID
NO:34),

5′-CAGGAAAAACAUAUGAACAAUUA-3′ (siAVIL2278, SEQ ID
NO:35),

5′-AGGAAAAACAUAUGAACAAUUAA-3′ (siAVIL2279, SEQ ID
NO:36),

5′-AAGCAAGAAGGCCUAUACCUAUU-3′ (siAVIL2285, SEQ ID
NO:37),

5′-UGCCAAAAAGGAGAAUUACCUCU-3′ (siAVIL2459, SEQ ID
NO:38),

5′-CUGGAAACAGCUCCAAAUGAAGA-3′ (siAVIL2543, SEQ ID
NO:39),

5′-AGCUCCAAAUGAAGAAAGAAAAG-3′ (siAVIL2551, SEQ ID
NO:40),

5′-AAGCAAGAAGGCCUAUACCUAUU-3′ (siAVIL2585, SEQ ID
NO:41),

5′-GAGCAGAUAGUGCCAAUAUCAGG-3′ (siAVIL2624, SEQ ID
NO:42),

5′-GGCAUGUUCUCCAUUUUUUCUCA-3′ (siAVIL2719, SEQ ID
NO:43),

5′-UGCAUUCCUUGCUUGUUAUAUAC-3′ (siAVIL2746, SEQ ID
NO:44),

5′-GUGCCAAUAUCAGGAAAUAAUUU-3′ (siAVIL2633, SEQ ID
NO:45),

5′-UGCCAAUAUCAGGAAAUAAUUUA-3′ (siAVIL2634, SEQ ID
NO:46),

5′-GCCAAUAUCAGGAAAUAAUUUAU-3′ (siAVIL2635, SEQ ID
NO:47),

5′-GCCUGACAUUCAGCUACUUAAUU-3′ (siAVIL2670, SEQ ID
NO:48),

5′-CUGACAUUCAGCUACUUAAUUUA-3′ (siAVIL2672, SEQ ID
NO:49),

5′-CAGCUACUUAAUUUAGAUAUAAU-3′ (siAVIL2680, SEQ ID
NO:50),

5′-AGCUACUUAAUUUAGAUAUAAUA-3′ (siAVIL2681, SEQ ID
NO:51),

5′-CUGCAAAUCACGGCAUGUUCUCC-3′ (siAVIL2708, SEQ ID
NO:52),

5′-UUGCUUGUUAUAUACCUAAAAUG-3′ (siAVIL2754, SEQ ID
NO:53),

5′-UGCUUGUUAUAUACCUAAAAUGU-3′ (siAVIL2755, SEQ ID
NO:54),

5′-UUGUUAUAUACCUAAAAUGUUAA-3′ (siAVIL2758, SEQ ID
NO:55),

5′-UACCUAAAAUGUUAACCAUAUAG-3′ (siAVIL2766, SEQ ID
NO:56),

5′-ACCUAAAAUGUUAACCAUAUAGU-3′ (siAVIL2767, SEQ ID
NO:57),

5′-AAGCACUAAAACUGCAUAAAUCU-3′ (siAVIL2829, SEQ ID
NO:58),

5′-UGCAUAAAUCUGGAGAAAUCAAA-3′ (siAVIL2841, SEQ ID
NO:59),

5′-CUGGAGAAAUCAAAAGAAAGAGA-3′ (siAVIL2850, SEQ ID
NO:60),

5′-GAGAAAUCAAAAGAAAGAGAACC-3′ (siAVIL2853, SEQ ID
NO:61),

5′-AAGAAAGAGAACCAAAAAACAAU-3′ (siAVIL2863, SEQ ID
NO:62),

5′-AACCAAAAAACAAUGCUUAAAAU-3′ (siAVIL2872, SEQ ID
NO:63),

5′-ACCAAAAAACAAUGCUUAAAAUG-3′ (siAVIL2873, SEQ ID
NO:64),

5′-AACAAUGCUUAAAAUGUUUAAUA-3′ (siAVIL2880, SEQ ID
NO:65),

5′-UGCUUAAAAUGUUUAAUAACUUU-3′ (siAVIL2885, SEQ ID
NO:66),

5′-AUGUUUAAUAACUUUAUGUUUAA-3′ (siAVIL2893, SEQ ID
NO:67),

5′-AACUUUAUGUUUAAUAUUAUACC-3′ (siAVIL2902, SEQ ID
NO:68),

5′-ACCUACCUUUGUUUUCAAUUUUA-3′ (siAVIL2928, SEQ ID
NO:69)

5′-UACCUUUGUUUUCAAUUUUAAGA-3′ (siAVIL2931, SEQ ID
NO:70),

5′-UUGUUUUCAAUUUUAAGAUGAUU-3′ (siAVIL2936, SEQ ID
NO:71),

5′-UUCAAUUUUAAGAUGAUUAUUUC-3′ (siAVIL2941, SEQ ID
NO:72),

5′-AUGAUUAUUUCUAAAAUCUAUUU-3′ (siAVIL2953, SEQ ID
NO:73),

5′-AUCUAUUUAGCCUGUAAAUCAUU-3′ (siAVIL2968, SEQ ID
NO:74),

5′-GCCUGUAAAUCAUUGAAAUCAUA-3′ (siAVIL2977, SEQ ID
NO:75),

5′-CUGUAAAUCAUUGAAAUCAUAUA-3′ (siAVIL2979, SEQ ID
NO:76),

5′-AUCAUUGAAAUCAUAUAUGCACU-3′ (siAVIL2985, SEQ ID
NO:77),

5′-UCCAAAUACCCAGAUCUGUAAUG-3′ (siAVIL3020, SEQ ID
NO:78),

5′-CUGUAAUGUGUCAAAGCAUUUUU-3′ (siAVIL3035, SEQ ID
NO:79),

5′-GUCAAAGCAUUUUUCACUUUUCA-3′ (siAVIL3044, SEQ ID
NO:80),

5′-AAGCAUUUUUCACUUUUCAAAUA-3′ (siAVIL3048, SEQ ID
NO:81),

5′-AGCAUUUUUCACUUUUCAAAUAA-3′ (siAVIL3049, SEQ ID
NO:82),

5′-UUCACUUUUCAAAUAAAGAUACC-3′ (siAVIL3056, SEQ ID
NO:83),

5′-UUCAAAUAAAGAUACCUAUAAUG-3′ (siAVIL3063, SEQ ID
NO:84),

5′-CCACACAGCUGGACGACUA-3′ (siAVIL265, SEQ ID NO:85
),

5′-GCUUCUGGCAAAGGAUAUU-3′ (siAVIL593, SEQ ID NO:86
),

5′-GCAAAGGAUAUUCGAGACA-3′ (siAVIL600, SEQ ID NO:87
),

5′-AUCAACUAUCAUGUUGUAUCAUA-3′ (siAVIL866, SEQ ID N
O:88),

5′-GCACCAAUGUGGAGACCGU-3′ (siAVIL1000, SEQ ID NO:8
9),

5′-GGAAAGAAUGGUCGAUGAU-3′ (siAVIL1187, SEQ ID NO:9
0),

5′-AAUUCAUGGAAAUGACAAAUCUA-3′ (siAVIL1673, SEQ ID
NO:91),

5′-UUACCUCUCUGAACAGGAC-3′ (siAVIL2474, SEQ ID NO:9
2),

5′-AACAGGACUUUGUGUCUGUGUU-3′ (siAVIL2485, SEQ ID N
O:93),

5′-GCAAGAAGGCCUAUACCUA-3′ (siAVIL2587, SEQ ID NO:9
4),

5′-GCAUUCCUUGCUUGUUAUA-3′ (siAVIL2651, SEQ ID NO:9
5),

5′-UAUUAUACCAGGACCUACC-3′ (siAVIL2916, SEQ ID NO:9
6), or

5′-GAGCUUAAGAAAAUGGAGC-3′ (siMARS-AVIL, SEQ ID NO:
97).

In some embodiments the silencing oligonucleotide is an shRNA comprising the nucleic acid sequence

5′-GATCCGCTTCTGGCAAAGGATATTTTCAAGAGAAATATCCTTTGCCA
GAAGCTTTTTG-3′ (shAVIL-593-S, SEQ ID NO:98),

5′-AATTCAAAAAGCTTCTGGCAAAGGATATTTCTCTTGAAAATATCCTT
TGCCAGAAGCG-3′ (shAVIL-593-AS, SEQ ID NO:99),

5′-GATCCGCATTCCTTGCTTGTTATATTCAAGAGATATAACAAGCAAGG
AATGCTTTTTG-3′ (shAVIL-2651-S, SEQ ID NO:100),

5′-AATTCAAAAAGCATTCCTTGCTTGTTATATCTCTTGAATATAACAAG
CAAGGAATGCG-3′ (shAVIL-2651-AS, SEQ ID NO:101),

5′-GCGCATCAACTATCATGTTGTATCATAGTGAAGCCACAGATGTATGA
TACAACATGATAGTTGATTTGC-3′ (Tet-induce shAVIL-866-S
, SEQ ID NO:102),

5′-GCAAATCAACTATCATGTTGTATCATACATCTGTGGCTTCACTATGA
TACAACATGATAGTTGATGCGC-3′ (Tet-induce shAVIL-866 A
S, SEQ ID NO:103),

5′-GCGCTTCATGGAAATGACAAATCTATAGTGAAGCCACAGATGTATAG
ATTTGTCATTTCCATGAATTGC-3′ (Tet-induce shAVIL-1673-
S, SEQ ID NO:104),

5′-GCAATTCATGGAAATGACAAATCTATACATCTGTGGCTTCACTATAG
ATTTGTCATTTCCATGAAGCGC-3′ (Tet-induce shAVIL-1673-
AS, SEQ ID NO:105),

5′-GCGCCAGGACTTTGTGTCTGTGTTATAGTGAAGCCACAGATGTATAA
CACAGACACAAAGTCCTGTTGC-3′ (Tet-induce shAVIL-2485-
S, SEQ ID NO:106), or

5′-GCAACAGGACTTTGTGTCTGTGTTATACATCTGTGGCTTCACTATAA
CACAGACACAAAGTCCTGGCGC-3′ (Tet-induce shAVIL-2485-
AS, SEQ ID NO:107).

In some embodiments the silencing oligonucleotide is an sgRNA comprising the nucleic acid sequence: 5′-CACCGTAGCAGCCGCTTCACGTCGT-3′ (AVILsgRNA1-A-F, SEQ ID NO:108), 5′-AAACACGACGTGAAGCGGCTGCTAC-3′ (AVILsgRNA1-A-R, SEQ ID NO:109), 5′-CACCGAGCTGGGACAGTTTCAACCG-3′ (AVILsgRNA1-B-F, SEQ ID NO:110), 5′-AAACCGGTTGAAACTGTCCCAGCTC-3′ (AVILsgRNA1-B-R, SEQ ID NO:111), 5′-CACCGAAGAAACATCAGGGCTACCG-3′ (AVILsgRNA2-A-F, SEQ ID NO:112), 5′-AAACCGGTAGCCCTGATGTTTCTTC-3′ (AVILsgRNA2-A-R, SEQ ID NO:113), 5′-CACCGCAACAGTGGGGAGCGCCTGA-3′ (AVILsgRNA2-B-F, SEQ ID NO:114), or 5′-AAACTCAGGCGCTCCCCACTGTTGC-3′ (AVILsgRNA2-B-R, SEQ ID NO:115).

In some embodiments the silencing oligonucleotide is an siRNA targeting the mRNA sequence AAAGCUGCGCAGCCAUAUAUACC (SEQ ID NO:145) and comprising the nucleic acid sequence: AGCUGCGCAGCCAUAUAUA (SEQ ID NO:123). In some embodiments the silencing oligonucleotide is an siRNA targeting the mRNA sequence AAUCACUGCUGACAUGAAGAAUG (SEQ ID NO:146) and comprising the nucleic acid sequence: UCACUGCUGACAUGAAGAA (SEQ ID NO:124). In some embodiments the silencing oligonucleotide is an siRNA targeting the mRNA sequence AAAACAGGCAGCCAUGUCUAAAG (SEQ ID NO:147) and comprising the nucleic acid sequence: AACAGGCAGCCAUGUCUAA (SEQ ID NO:125). In some embodiments the silencing oligonucleotide is an siRNA targeting the mRNA sequence AAGUUGAGGUCUGGAGAAUUGAG (SEQ ID NO:148) and comprising the nucleic acid sequence: GUUGAGGUCUGGAGAAUUG (SEQ ID NO:126). In some embodiments the silencing oligonucleotide is an siRNA targeting the mRNA sequence AAGGAGAAUUACCUCUCUGAACA (SEQ ID NO:149) and comprising the nucleic acid sequence: GGAGAAUUACCUCUCUGAA (SEQ ID NO:127). In some embodiments the silencing oligonucleotide is an siRNA targeting the mRNA sequence AAGCAAGAAGGCCUAUACCUAUU (SEQ ID NO:150) and comprising the nucleic acid sequence: GCAAGAAGGCCUAUACCUA (SEQ ID NO:128). In some embodiments the silencing oligonucleotide is an siRNA targeting the mRNA sequence AAGAAGGCCUAUACCUAUUGCAA (SEQ ID NO:151) and comprising the nucleic acid sequence: GAAGGCCUAUACCUAUUGC (SEQ ID NO:129). In some embodiments the silencing oligonucleotide is an siRNA targeting the mRNA sequence AAAGAGCAGAUAGUGCCAAUAUC (SEQ ID NO:152) and comprising the nucleic acid sequence: AGAGCAGAUAGUGCCAAUA (SEQ ID NO:130). In some embodiments the silencing oligonucleotide is an siRNA targeting the mRNA sequence AAGCUGCGCAGCCAUAUAUACCA (SEQ ID NO:153) and comprising the nucleic acid sequence: GCUGCGCAGCCAUAUAUAC (SEQ ID NO:131). In some embodiments the silencing oligonucleotide is an siRNA targeting the mRNA sequence GAAGCACGUGGAGACCAAUACCU (SEQ ID NO:154) and comprising the nucleic acid sequence: AGCACGUGGAGACCAAUAC (SEQ ID NO:132).

In some embodiments the silencing oligonucleotide is an siRNA targeting the mRNA sequence GCAGAAATCAACTATCATG (SEQ ID NO:155) and comprising the nucleic acid sequence: GCAGAAAUCAACUAUCAUG (sense, SEQ ID NO:133) or CGUCUUUAGUUGAUAGUAC (antisense, SEQ ID NO:134). In some embodiments the silencing oligonucleotide is an siRNA targeting the mRNA sequence CAGGACTTTGTGTCTGTGT (SEQ ID NO:156) and comprising the nucleic acid sequence: CAGGACUUUGUGUCUGUGU (sense, SEQ ID NO:135) or GUCCUGAAACACAGACACA (antisense, SEQ ID NO:136). In some embodiments the silencing oligonucleotide is an siRNA targeting the mRNA sequence GCAAGAAGGCCTATACCTA (SEQ ID NO:157) and comprising the nucleic acid sequence: GCAAGAAGGCCUAUACCUA (sense, SEQ ID NO:137) or CGUUCUUCCGGAUAUGGAU (antisense, SEQ ID NO:138). In some embodiments the silencing oligonucleotide is an siRNA targeting the mRNA sequence ATCACGGCATGTTCTCCAT (SEQ ID NO:158) and comprising the nucleic acid sequence: AUCACGGCAUGUUCUCCAU (sense, SEQ ID NO:139) or UAGUGCCGUACAAGAGGUA (antisense, SEQ ID NO:140). In some embodiments the silencing oligonucleotide is an siRNA targeting the mRNA sequence TTACCTCTCTGAACAGGAC (SEQ ID NO:159) and comprising the nucleic acid sequence: UUACCUCUCUGAACAGGAC (sense, SEQ ID NO:141) or AAUGGAGAGACUUGUCCUG (antisense, SEQ ID NO:142). In some embodiments the silencing oligonucleotide is an siRNA targeting the mRNA sequence TATTATACCAGGACCTACC (SEQ ID NO:160) and comprising the nucleic acid sequence: UAUUAUACCAGGACCUACC (sense, SEQ ID NO:143) or AUAAUAUGGUCCUGGAUGG (antisense, SEQ ID NO:144).

### Pharmaceutical Compositions

In various aspects, the present disclosure relates to pharmaceutical compositions comprising a therapeutically effective amount of at least one disclosed silencing oligonucleotide, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed silencing oligonucleotide, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a silencing oligonucleotide of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed silencing oligonucleotide, or a pharmaceutically acceptable salt thereof. In a further aspect, a disclosed silencing oligonucleotide, or pharmaceutically acceptable salt thereof, can also be included in a pharmaceutical composition in combination with one or more other therapeutically active silencing oligonucleotides. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington’s Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The silencing oligonucleotides described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable silencing oligonucleotide will be in a form suitable for oral, rectal, topical, intravenous injection or parenteral administration. Carriers include solids or liquids, and the type of carrier is chosen based on the type of administration being used. The silencing oligonucleotides may be administered as a dosage that has a known quantity of the silencing oligonucleotides.

Pharmaceutical compositions of the present disclosure suitable injection, such as parenteral administration, such as intravenous, intramuscular, or subcutaneous administration. Pharmaceutical compositions for injection can be prepared as solutions or suspensions of the silencing oligonucleotides in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer’s dextrose, dextrose and sodium chloride, lactated Ringer’s and fixed oils. Intravenous vehicles include fluid and nutrient replenishers, electrolyte replenishers such as those based on Ringer’s dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require regulation, limitation, or inhibition of AVIL activity an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The silencing oligonucleotides can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific silencing oligonucleotide employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for regulating, limiting or inhibiting AVIL activity (e.g., treatment of one or more disorders associated with AVIL dysfunction) in mammals (e.g., humans) comprising combining one or more disclosed silencing oligonucleotides, products, or compositions with a pharmaceutically acceptable carrier or diluent. Thus, in one aspect, the present disclosure further relates to a method for manufacturing a medicament comprising combining at least one disclosed silencing oligonucleotide or at least one disclosed product with a pharmaceutically acceptable carrier or diluent.

The disclosed pharmaceutical compositions can further comprise other therapeutically active silencing oligonucleotides, which are usually applied in the treatment of the above mentioned pathological or clinical conditions.

It is understood that the disclosed compositions can be prepared from the disclosed silencing oligonucleotides. It is also understood that the disclosed compositions can be employed in the disclosed methods of using.

As already mentioned, the present disclosure relates to a pharmaceutical composition comprising a therapeutically effective amount of a disclosed silencing oligonucleotide, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a silencing oligonucleotide according to the present disclosure.

### Methods of Using the Silencing Oligonucleotides

In a further aspect, the present disclosure provides methods of treatment comprising administration of a therapeutically effective amount of a disclosed pharmaceutical composition as disclosed herein above to a subject in need thereof. In particular, the disclosed silencing oligonucleotides and disclosed pharmaceutical compositions can be used in methods of treating a disease or disorder that is associated with increased, aberrant, or dysfunctional levels of advillin (AVIL) activity in a cell, tissue, or organism. That is, the disclosed silencing oligonucleotides and disclosed pharmaceutical compositions can be used to regulate, limit, or inhibit AVIL activity in a cell, tissue, or organism to provide a clinical or therapeutic benefit to a subject which has been determined to or been diagnosed to have with increased, aberrant, or dysfunctional levels of AVIL activity.

Adult cancers often have complex genomic landscapes, making it challenging to identify key cancer-driving events. As disclosed herein below, a pediatric rhabdomyosarcoma was identified as having a gene fusion, AVIL fused to a house-keeping gene, MARS. In adults, AVIL was overexpressed in all of the glioblastomas tested herein below. Tumors were addicted to AVIL dysregulation: silencing the MARS-AVIL fusion in rhabdomyosarcoma, or silencing AVIL in glioblastoma nearly eradicated the cells in culture, and dramatically inhibited in vivo xenografts in mice. Conversely, overexpressing AVIL promoted tumorigenesis. GBM and lower-grade glioma patients with increased AVIL expression had worse prognosis. The effect of AVIL was partly mediated by LIN28B, whose expression also correlated with clinical prognosis. High levels of AVIL expression were also associated with poor patient outcomes in several other cancers.

Oncogene addiction describes a phenomenon according to which tumor cells become reliant on the activity of a particular oncogene and die once this activity is inhibited. (Vivanco, 2014; Weinstein, 2002; Weinstein and Joe, 2006). Many of the targeted cancer therapies exploit this concept (Lord and Ashworth, 2013; Luo et al., 2009). It is perhaps best exemplified by the successful use of imatinib in the therapy of chronic myelogenous leukemia (CML) (Druker et al., 2001). In CML, the major driver of tumorigenesis is the BCR-ABL fusion oncogene; imatinib inhibits the constitutively active BCR-ABL protein kinase, to which leukemic cells become addicted. Other successful examples include trastuzumab targeting ERBB2 addiction (Paik et al., 2008), and vemurafenib targeting BRAF addiction (Bollag et al., 2010; Chapman et al., 2011; Davies et al., 2002). The challenge is to find such key oncogenes. Even though large sets of genome and transcriptome data are available to facilitate the identification of driver mutations in cancer, true signals are often buried in a large number of passenger events.

Glioblastoma (GBM) is the most common primary brain tumor and among the deadliest of human cancers. Despite advances in surgery, radiation and chemotherapy, survival of patients affected by GBM remains dismal (^{~}15 months after diagnosis). (Prados and Levin, 2000; Castro et al., 2003; King et al., 2005; Stupp et al., 2005). Clearly, better treatment options, and identification of novel therapeutic targets are urgently needed.

Tumor cells use multiple “tricks” to dysregulate some oncogenes, which at the same time give credence to the genes as key players in tumorigenesis and malignancy. However, this knowledge is usually accumulated over a long period of time and often involves different laboratories examining various types of cancer. Our strategy is to use this concept proactively to find key oncogenes that are dysregulated by multiple mechanisms in different types of cancer. Most adult solid tumors have a complex landscape of genetic lesions, impeding analysis. In contrast, pediatric tumors tend to have fewer point mutations and structural changes. Our study was initiated in the pediatric tumor, rhabdomyosarcoma. As disclosed herein below, a gene fusion has been described which results in the juxtaposition of a house-keeping gene next to the AVIL gene, in particular, it is disclosed herein below that a subset of GBMs retain AVIL amplification. Interestingly, at RNA and protein levels, all of the GBM cases in our collection overexpress AVIL. Loss-of-function experiments proved the dependency of tumor growth on AVIL dysregulation, yet no effect on non-cancer astrocytes was observed. Consistently, forced overexpression of AVIL resulted in enhanced tumorigenesis. Clinically, higher expression of AVIL correlates with worse patient outcome in GBMs as well as in lower-grade gliomas. The oncogenic effect is at least partly mediated by LIN28B in gliomas. In addition to gliomas, lung cancer, bladder cancer, and renal cancer patients with high level of AVIL expression also had worse prognosis.

Glioblastoma (GBM), WHO classification Grade IV Astrocytoma, is the most common, and most aggressive malignant primary brain tumor in humans (Dunn et al., 2012). Survival of patients affected by GBM has remained low, despite advances in surgery, radiation, and chemotherapy (Castro et al., 2003; King et al., 2005; Prados and Levin, 2000; Stupp et al., 2005). About 50% of patients diagnosed with GBM die within one year, and 90% die within three years (American Brain Tumor Association, 2014). The disclosure herein below shows that AVIL is overexpressed in the vast majority, if not all of human glioblastomas. Moreover, it was found that GBM cells depend on the overexpression of AVIL for increased survival and migration. Silencing AVIL induced GBM cell death in vitro, and prevented GBM xenograft formation and growth in animal models. Silencing AVIL also dramatically changed cell morphology, and reduced cell migration/invasion ability. In contrast, normal astrocytes express very low levels of AVIL, and silencing AVIL had no obvious effect on cell growth, or morphology. Taken together, this demonstrates that AVIL is a new and promising selective therapeutic target, inhibition of which may effectively suppress GBM growth and invasion, yet spare normal brain cells.

In some aspects, the method includes diagnosis of the subject’s need for treatment prior to administration of the silencing oligonucleotide or pharmaceutical composition. In some aspects, the subject has been diagnosed with a disorder treatable by regulation, limitation, or inhibition of AVIL prior to administering. In some aspects, the subject has been diagnosed with a cancer. In some aspects, the method includes identifying a subject’s need for treatment prior to the administering step.

The disclosed silencing oligonucleotides can be used as single agents or in combination with one or more other drugs in the treatment, prevention, control, amelioration or reduction of risk of the aforementioned diseases, disorders and conditions for which silencing oligonucleotides have utility, where the combination of drugs together are safer or more effective than either drug alone. The other drug(s) can be administered by a route and in an amount commonly used therefore, contemporaneously or sequentially with a disclosed silencing oligonucleotide. When a disclosed silencing oligonucleotide is used contemporaneously with one or more other drugs, a pharmaceutical composition in unit dosage form containing such drugs and the disclosed silencing oligonucleotide is preferred. However, the combination therapy can also be administered on overlapping schedules. It is also envisioned that the combination of one or more active ingredients and a disclosed silencing oligonucleotide will be more efficacious than either as a single agent.

AVIL is known as a member of the vilin/gelsolin family, which regulates actin filament reorganization (Marks et al., 1998). In encodes a protein also called advillin, which is known to affect cell movement and has been reported to be involved in the formation of filopedia-like structures in fibroblasts, as well as a role in ciliogenesis (Morin et al., 2010). AVIL is overexpressed in in many, if not all, glioblastomas. GBM cells depend on the overexpression of AVIL for increased survival and migration. Silencing AVIL induced GBM cell death in vitro, and can prevent GBM xenograft formation and growth in animal models. Silencing AVIL can also change cell morphology in GBM cells, and reduce cell migration/invasion ability. In contrast, normal astrocytes express very low levels of AVIL, and silencing AVIL had no obvious effect on cell growth, or morphology.

Therefore, silencing oligonucleotide that regulate, limit, or inhibit AVIL expression show improved prognosis in the treatment of cancers in which there is an inverse correlation between AVIL expression and patient prognosis, such as, but not limited to, brain cancer and cancerous tumors such as glioblastomas, rhabdosarcomas, gliomas, lung cancer, bladder cancer including bladder urothelial carcinoma, and renal cancer including kidney clear cell carcinoma.

Accordingly, in various aspects, the present disclosure pertains to methods of targeting AVIL with the disclosed silencing oligonucleotides. The disclosed silencing oligonucleotides or disclosed pharmaceutical compositions can act as regulators of AVIL expression in cells having increased, aberrant, or dysfunctional levels of AVIL, and accordingly can be useful in the treatment of cancer (including but not limited to those types mentioned herein).

The compositions can be administered alone or combination with a chemotherapeutic drug. Combination therapy can present advantages over single-agent therapies: lower treatment failure rate, lower case-fatality ratios, slower development of resistance and consequently, less money needed for the development of new drugs. Chemotherapeutic drugs include conventional chemotherapeutic reagents such as alkylating agents, anti-metabolites, anti-mitototics, plant alkaloids, antibiotics, and miscellaneous compounds. Examples of these drugs include CDDP, methotrexate, vincristine, adriamycin, bleomycin, carmustine, hydroxyurea, hydrazine, nitrosoureas, triazenes such as dacarabzine and temozolomide, nitrogen mustards such as chlorambucil, cyclophosphamide, isofamide, mechlorethamine, melphalan, uracil mustard; aziridine such as thiotepa; methanesulphonate esters such as busulfan; platinum complexes such as cisplatin, carboplatin; bioreductive alkylators, such as mitomycin and and altretemine. Chemotherapeutic drugs also include proteasome inhibitors such as salinosporamides, bortezomib, PS-519, and omuralide. The disclosed silencing oligonucleotides can also be administered in combination with surgery. For example, the disclosed silencing oligonucleotides can be administered prior to, during or after surgery or radiotherapy. Adminstration during surgery can be as a bathing solution for the operation site. The resected tumor can also be bathed in the disclosed silencing oligonucleotides.

In further aspects, the disclosed silencing oligonucleotides can be utilized in combination with one or more chemotherapeutic agents. For example, the one or more chemotherapeutic agent can be a chemotherapeutic agent selected from alkylating agents, antimetabolites, platinating agents, toxoids, EGFR inhibitors, anti-hormonal agents, topoisomerase inhibitors, tubulin agents, signaling inhibitors (e.g., kinase inhibitors), and other chemotherapeutic agents.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1l and calicheamicin omegal1 (see, e.g., Agnew, Chem Intl. Ed. Engl., 33:183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; dacarbazine; mannomustine; mitobronitol; mitolactol; pipobroman; gacytosine; arabinoside (“Ara-C″); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin, and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovovin; novantrone; edatrexate; daunomycin; aminopterin; ibandronate; topoisomerase inhibitor RFS 2000; difluorometlhylornithine (DMFO); retinoids such as retinoic acid, including bexarotene (TARGRETIN®); bisphosphonates such as clodronate (for example, BONEFOSO® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN®) combined with 5-FU and leucovovin.

Herein, chemotherapeutic agents include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

Herein, a “taxoid” is a chemotherapeutic agent that functions to inhibit microtubule depolymerization. Examples include paclitaxel (TAXOL®), albumin-engineered nanoparticle formulation of paclitaxel (ABRAXANE™), and docetaxel (TAXOTERE®). The preferred taxoid is paclitaxel.

As used herein, the term “EGFR inhibitor” refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225(C225 or Cetuximab; ERBUTlX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al., Eur. J. Cancer, 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem., 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos: 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N44-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA J) 4-(3′-Chloro-4′-fluoroanilino)-7-methoxy-6-(3-morpholino propoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)- pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Sugen); AG1571 (SU 5271; Sugen); dual EGFR/HER2 tyrosine kinase inhibitors such as lapatinib (GW 572016 or N-[3-chloro-4-[(3fluorophenyl)methoxy]phenyl]6[5[[[2methylsulfonyl) ethyl]amino]methyl]-2-furanyl]-4-quinazolinamine; Glaxo-SmithKline).

A “tyrosine kinase inhibitor” is a molecule which inhibits tyrosine kinase activity of a tyrosine kinase such as a HER receptor. Examples of such inhibitors include the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GW572016; available from Glaxo-SmithKline) an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibits Raf-1 signaling; non-HER targeted TK inhibitors such as Imatinib mesylate (GLEEVAC J) available from Glaxo; MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); Imatinib mesylate (Gleevac; Novartis); PKI 166 (Novartis); GW2016 (Glaxo SmithKline); CI-1033 (Pfizer); EKB-569 (Wyeth); Semaxinib (Sugen); ZD6474 (AstraZeneca); PTK-787 (Novartis/Schering AG); INC-1C11 (Imclone); or as described in any of the following patent publications: U.S. Pat. No. 5,804,396; WO99/09016 (American Cyanamid); WO98/43960 (American Cyanamid); WO97/38983 (Warner Lambert); WO99/06378 (Warner Lambert); WO99/06396 (Warner Lambert); WO96/30347 (Pfizer, Inc); WO96/33978 (Zeneca); WO96/3397 (Zeneca); and WO96/33980 (Zeneca).

Thus in various further aspects, the disclosed silencing oligonucleotides can be administered to subjects in combination with one or more chemotherapeutic drugs. For example, treating a subject with a glioma can be affected by a method comprising administering to the subject a disclosed silencing oligonucleotide or a pharmaceutically acceptable salt or hydrate thereof in combination with one or more chemotherapeutic drugs. For example, inhibiting intracranial metastasis of gliomal cancer cells in a subject can be effected by a method comprising administering to the subject a disclosed silencing oligonucleotide or a pharmaceutically acceptable salt or hydrate thereof in combination with one or more chemotherapeutic drugs. For example, preventing relapse of glioma in a subject can be effected by a method comprising administering to the subject a disclosed silencing oligonucleotide or a pharmaceutically acceptable salt or hydrate thereof in combination with one or more chemotherapeutic drugs.

It is contemplated that the disclosed silencing oligonucleotides can be administered before, simultaneously, or after the adminstration of one or more chemotherapeutic drugs. While not wishing to be bound by theory, it is believed that the disclosed silencing oligonucleotides, in combination with one or more chemotherapeutic drugs, can have an augmented or synergystic effect on the subject. Further, disclosed silencing oligonucleotides, in combination with one or more chemotherapeutic drugs, can be individually given in dosages lower than the one or more chemotherapeutic drugs would be typically administered as single-agent therapies.

In further aspects, the invention relates to adminstration of the disclosed silencing oligonucleotides to subjects in combination with temozolomide (4-methyl-5-oxo-2,3,4,6,8-pentazabicyclo [4.3.0]nona-2,7,9-triene-9-carboxamide). For example, treating a subject with a glioma can be affected by a method comprising administering to the subject a disclosed silencing oligonucleotide or a pharmaceutically acceptable salt or hydrate thereof in combination with temozolomide. For example, inhibiting intracranial metastasis of gliomal cancer cells in a subject can be affected by a method comprising administering to the subject a disclosed silencing oligonucleotide or a pharmaceutically acceptable salt or hydrate thereof in combination with temozolomide. For example, preventing relapse of glioma in a subject can be affected by a method comprising administering to the subject a disclosed silencing oligonucleotide in combination with Temozolomide.

It is also understood that the disclosed silencing oligonucleotides, when administered to subjects in combination with one or more chemotherapeutic drugs, can also be employed in connection with radiation therapy and/or surgical therapy.

Radiation therapy (Radiotherapy), including brachytherapy, can be used to treat gliomas. In one aspect, the invention relates to the adminstration of the disclosed silencing oligonucleotides to subjects in connection with radiation therapy. It is contemplated that the disclosed silencing oligonucleotides can be administered before, during, or after the radiation therapy. For example, treating a subject with a glioma can be affected by a method comprising administering to the subject a disclosed silencing oligonucleotide or a pharmaceutically acceptable salt or hydrate thereof in connection with radiation therapy. For example, inhibiting intracranial metastasis of gliomal cancer cells in a subject can be affected by a method comprising administering to the subject a disclosed silencing oligonucleotide in connection with radiation therapy. For example, preventing relapse of glioma in a subject can be affected by a method comprising administering to the subject a disclosed silencing oligonucleotide in connection with radiation therapy.

While not wishing to be bound by theory, it is believed that the disclosed silencing oligonucleotides, in combination with radiotherapy, can have an augmented or synergystic effect in a subject. Further, the disclosed silencing oligonucleotides, when used in combination with radiotherapy, can lower a subject’s need for radiotherapy (e.g., less radiation need be used) and/or can lower a subject’s need for disclosed silencing oligonucleotides (e.g., disclosed silencing oligonucleotides can be given in dosages lower than would be typically administered as single-agent therapies).

It is also understood that the disclosed silencing oligonucleotides, when administered to subjects in connection with radiation therapy, can also be employed in combination with one or more chemotherapeutic drugs and/or in connection surgical therapy.

Surgery can be used to treat gliomas. In one aspect, the invention relates to the adminstration of the disclosed silencing oligonucleotides to subjects in connection with surgical treatment. For example, treating a subject with a glioma can be effected by a method comprising administering to the subject a disclosed silencing oligonucleotide in connection with surgery. For example, inhibiting intracranial metastasis of gliomal cancer cells in a subject can be effected by a method comprising administering to the subject a disclosed silencing oligonucleotide in connection with surgery. For example, preventing relapse of glioma in a subject can be effected by a method comprising administering to the subject a disclosed silencing oligonucleotide in connection with surgery.

It is contemplated that the disclosed silencing oligonucleotides can be administered before, during, or after surgical treatment. While not wishing to be bound by theory, it is believed that the disclosed silencing oligonucleotides, in combination with surgery, can have an augmented or synergystic effect on the subject. Further, disclosed silencing oligonucleotides, when used in combination with surgery, can lower a subject’s need for surgery (e.g., less tissue need be removed) and/or can lower a subject’s need for disclosed silencing oligonucleotides (e.g., disclosed silencing oligonucleotides can be given in dosages lower than would be typically administered as single-agent therapies).

It is also understood that the disclosed silencing oligonucleotides, when administered to subjects in connection with surgical therapy, can also be employed in connection with radiation therapy and/or surgical therapy.

The disclosed compositions can also be employed to prevent relapse in a subject previously treated for a glioma. In one aspect, such a method comprises administering to the subject a prophylactically effective amount of a disclosed silencing oligonucleotide. It is understood that the dosage needed to prevent relapse (i.e. maintenance dose) may be less (e.g., half) of the dosage needed to effect treatment of a glioma.

It is also understood that when using the disclosed silencing oligonucleotides for preventing of relapse of glioma, in either single agent therapy or in combination therapy, can be also administered to subjects in connection with surgical therapy and/or surgical therapy.

In a further aspect, the cancer is a cancer of the brain. In a still further aspect, the cancer is selected from acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor cell.

In a further aspect, the cancer is a glioma. In a still further aspect, the glioma is glioblastoma multiforme. In a yet further aspect, the glioma is selected from is selected from a ependymoma, astrocytoma, oligodendroglioma, and oligoastrocytoma. In a yet further aspect, the glioma is selected from a juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, paraganglioma, and ganglioglioma cell.

Cancers that may be treated include, but are not limited to: cardiac; sarcoma (angiosarcoma, fibrosarcoma, rhabdomyosarcoma, liposarcoma); myxoma; rhabdomyoma; fibroma; lipoma and teratoma; lung, e.g., non-small cell lung, bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal, e.g., esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi’s sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; genitourinary tract, e.g., kidney (adenocarcinoma, Wilm’s tumor [nephroblastoma], lymphoma, leukemia), bladder and urethra (squamous cell carcinoma, transitional cell carcinoma, adenocarcinoma), prostate (adenocarcinoma, sarcoma), testis (sem inoma, teratoma, embryonal carcinoma, teratocarcinoma, choriocarcinoma, sarcoma, interstitial cell carcinoma, fibroma, fibroadenoma, adenomatoid tumors, lipoma); liver, e.g., hepatoma (hepatocellular carcinoma), cholangiocarcinoma, hepatoblastoma, angiosarcoma, hepatocellular adenoma, hemangioma; bone, e.g., osteogenic sarcoma (osteosarcoma), fibrosarcoma, malignant fibrous histiocytoma, chondrosarcoma, Ewing’s sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, e.g., skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); gynecological, e.g., uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); hematologic, e.g., blood (myeloid leukemia [acute and chronic], acute lymphoblastic leukemia, chronic lymphocytic leukemia, myeloproliferative diseases, multiple myeloma, myelodysplastic syndrome), Hodgkin’s disease, non-Hodgkin’s lymphoma [malignant lymphoma]; skin, e.g., malignant melanoma, basal cell carcinoma, squamous cell carcinoma, Karposi’s sarcoma, moles dysplastic nevi, lipoma, angioma, dermatofibroma, keloids, psoriasis; and adrenal glands, e.g., a neuroblastoma. Thus, the term “cancerous cell” as provided herein, includes a cell afflicted by any one of the above-identified conditions.

Cancers that may be treated include, but are not limited to, any of the following cancers in which it has been determined that the cancer is associated with aberrant expression of AVIL: cardiac; sarcoma (angiosarcoma, fibrosarcoma, rhabdomyosarcoma, liposarcoma); myxoma; rhabdomyoma; fibroma; lipoma and teratoma; lung, e.g., non-small cell lung, bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal, e.g., esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi’s sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; genitourinary tract, e.g., kidney (adenocarcinoma, Wilm’s tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, e.g., hepatoma (hepatocellular carcinoma), cholangiocarcinoma, hepatoblastoma, angiosarcoma, hepatocellular adenoma, hemangioma; bone, e.g., osteogenic sarcoma (osteosarcoma), fibrosarcoma, malignant fibrous histiocytoma, chondrosarcoma, Ewing’s sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, e.g., skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); gynecological, e.g., uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); hematologic, e.g., blood (myeloid leukemia [acute and chronic], acute lymphoblastic leukemia, chronic lymphocytic leukemia, myeloproliferative diseases, multiple myeloma, myelodysplastic syndrome), Hodgkin’s disease, non-Hodgkin’s lymphoma [malignant lymphoma]; skin, e.g., malignant melanoma, basal cell carcinoma, squamous cell carcinoma, Karposi’s sarcoma, moles dysplastic nevi, lipoma, angioma, dermatofibroma, keloids, psoriasis; and adrenal glands, e.g., a neuroblastoma. Thus, the term “cancerous cell” as provided herein, includes a cell afflicted by any one of the above-identified conditions.

### Kits

In a further aspect, the present disclosure relates to kits comprising at least one disclosed silencing oligonucleotides, and one or more of: (a) at least one agent known to treat a cancer; (b) instructions for treating a cancer and/or instructions for administering the silencing oligonucleotide in connection with the treatment of cancer. The disclosed silencing oligonucleotide can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual silencing oligonucleotides may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise silencing oligonucleotides and/or products co-packaged, co-formulated, and/or co-delivered with other components.

It is contemplated that the disclosed kits can be used in connection with the disclosed methods of making, the disclosed methods of using or treating, and/or the disclosed compositions.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Results

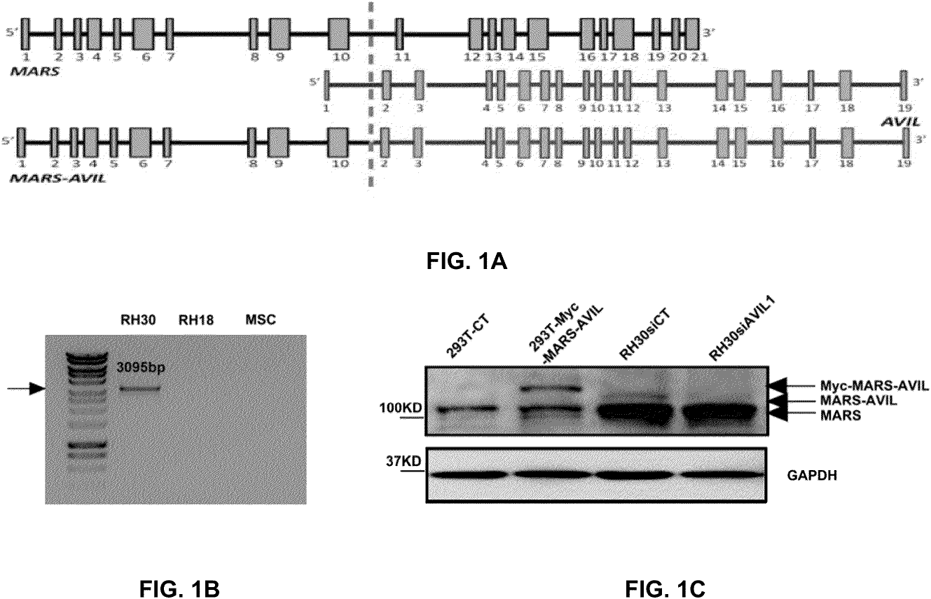

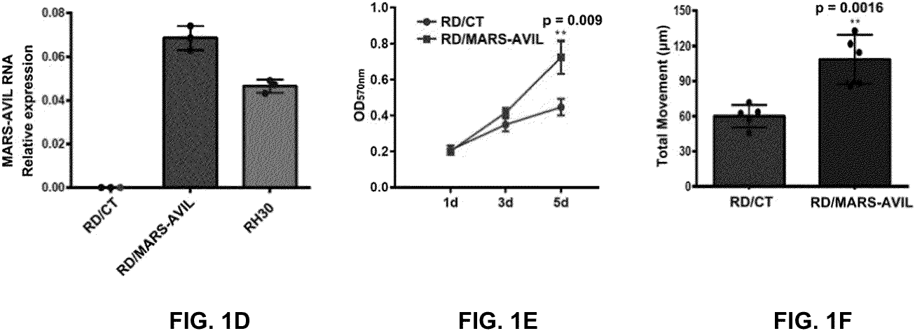

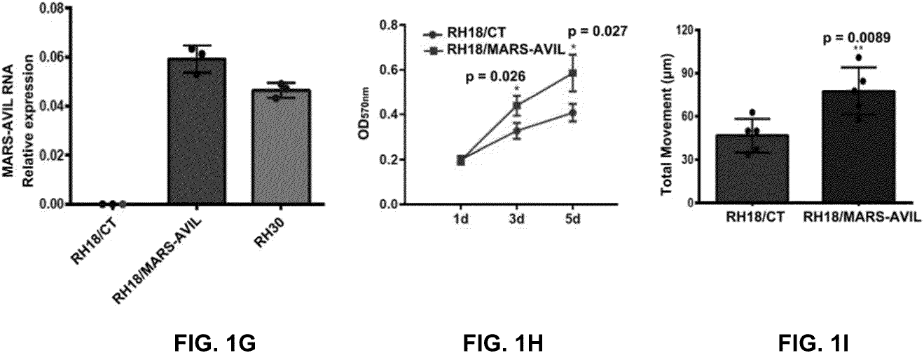

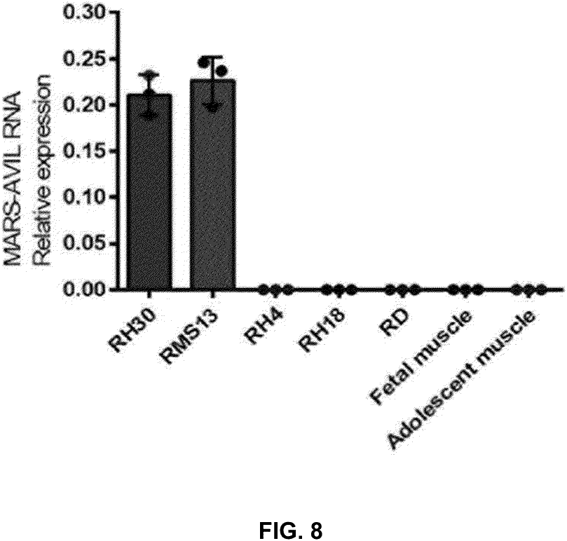

MARS-AVIL fusion encodes a fusion protein in RMS. Through paired-end RNA-sequencing, a fusion transcript joining the first ten exons of MARS (methionyl-tRNA synthetase) to the last 15 exons of AVIL (advillin) was identified in RH30 (RMS cell line), (FIG. 1A). Surprisingly, even though PAX3-FOXO1 is the most well-known fusion in the rhabdomyosarcomas of this type (ARMS), MARS-AVIL has the highest number of reads. The same MARS-AVIL fusion was also found in RMS-13, which is thought to be derived from the same donor (Hinson, A. R. et al. Front Oncol 2013 3:183), although it has a different fusion RNA profile from RH30. Other RMS lines, RH4, RH18, RD, and an Ewing sarcoma line A673, as well as fetal and adolescence muscles, do not harbor the same fusion (FIGS. 1A and 8). The MARS and AVIL genes are located in the same chromosomal region, 12q14, with 300kb and about 15 genes separating them. They transcribe in a head-to-head configuration. Using long-range PCR, it was determined that the fusion transcript results from an inversion of a fragment covering exon10 of MARS and exon1 of AVIL (FIG. 1A), resulting in a head-to-tail configuration. With Sanger sequencing, the break site was pinpointed, which is located in intron9 of MARS and intron1 of AVIL. Long-range PCR amplified the rearranged fragment in RH30, but not in another rhabdomyosarcoma cell line, RH18, or a mesenchymal stem cell culture (MSC) (FIG. 1B). Interestingly, an Image clone BC004134 (Strausberg, R. L. et al. Proc Natl Acad Sci U S A 2002 99:16899-16903), with a 100% match to the fusion junction, has been deposited in the human non-reference RNA database. It was labeled as an “mRNA similar to advillin”, and the tissue source was “rhabdomyosarcoma”, indicating that the fusion transcript was found independently before, just not recognized as a fusion from a chromosomal rearrangement.

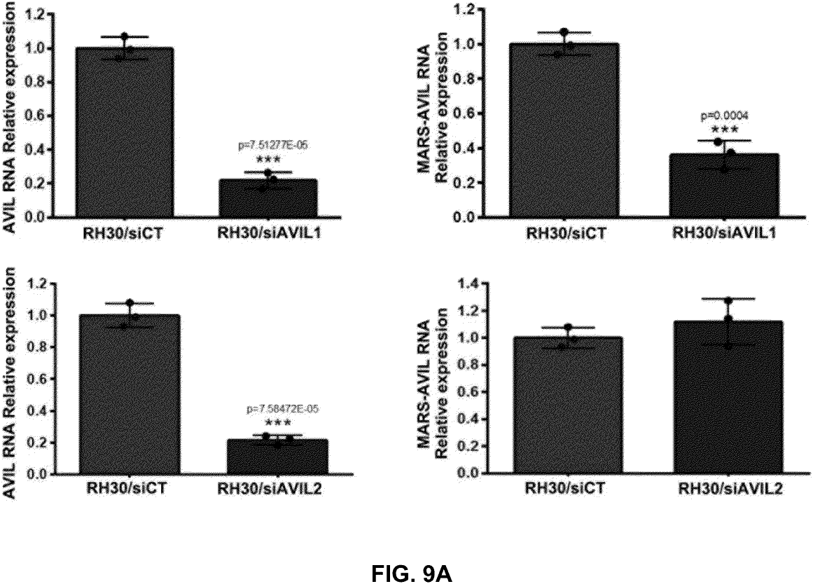

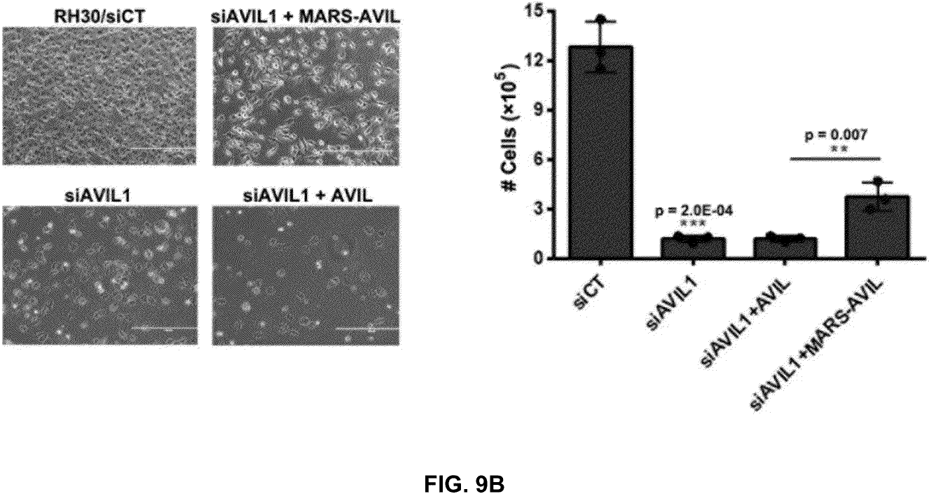

The reading frame of the AVIL portion is the same as the MARS portion, predicting that the fusion transcript will translate into an in-frame chimeric protein. Using a MARS antibody, a correctly-sized protein was detected as the possible fusion protein. To prove the identity of the band, a siRNA targeting the 3′ of AVIL was used, which silenced both AVIL and MARS-AVIL (FIG. 9A) and the reduction of the MARS-AVIL protein signal with the MARS antibody was detected. The same antibody also detected a Myc-tagged MARS-AVIL fusion in a 293T system (FIG. 1C).

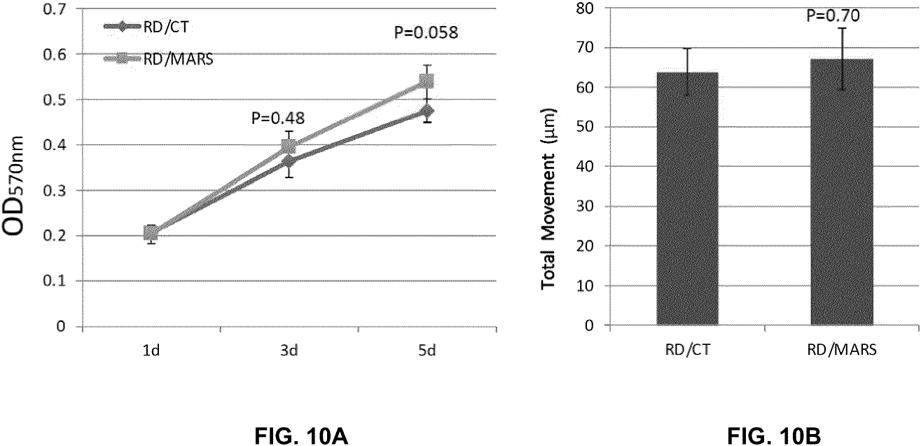

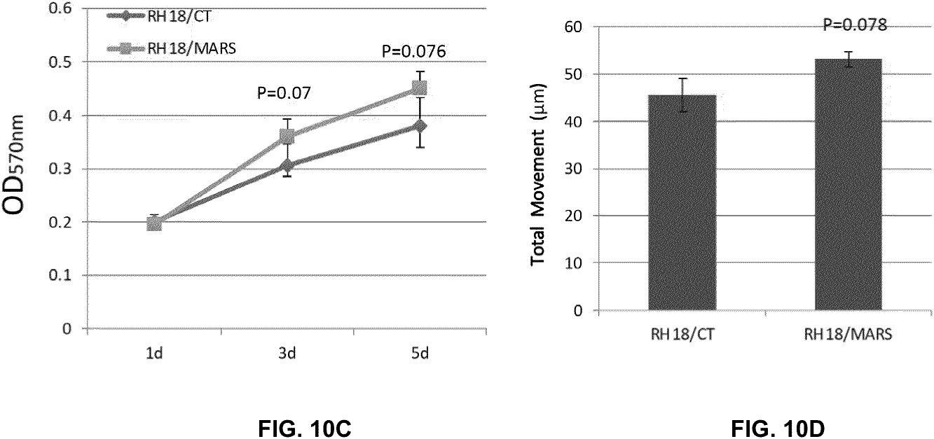

To investigate the role of MARS-AVIL fusion in RMS, RD and RH18 cells (both negative for MARS-AVIL) were generated that stably express the fusion to a level similar to that of RH30 (FIGS. 1D and 1G). In both cell types, the fusion-transfected cells had significantly higher growth rates (FIGS. 1E and 1H) and motility measured by wound healing assays (FIGS. 1F and 1I). In contrast, wild type MARS was overexpressed in the same two lines, and observed no statistically significant difference in cell growth and migration (FIG. 10).

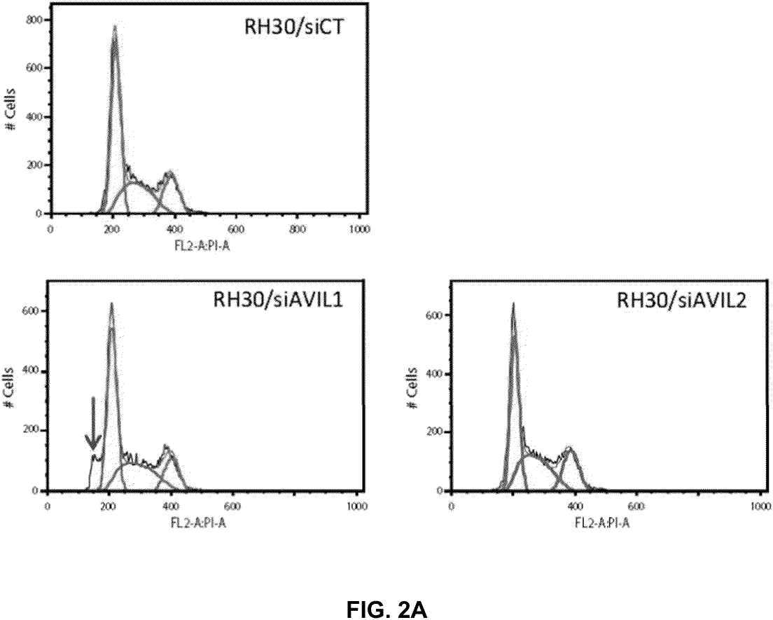

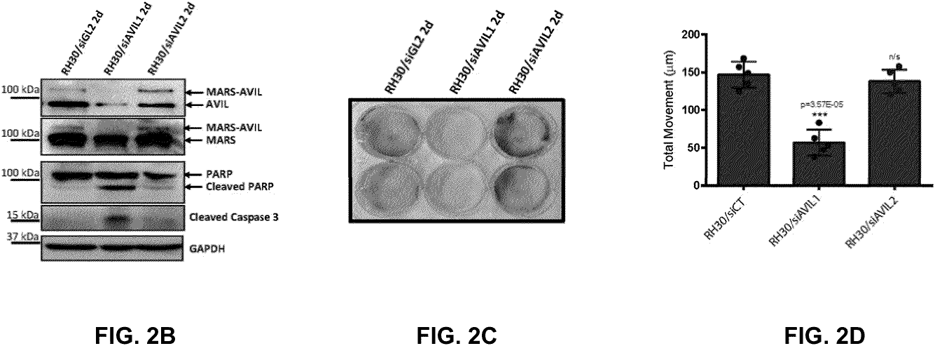

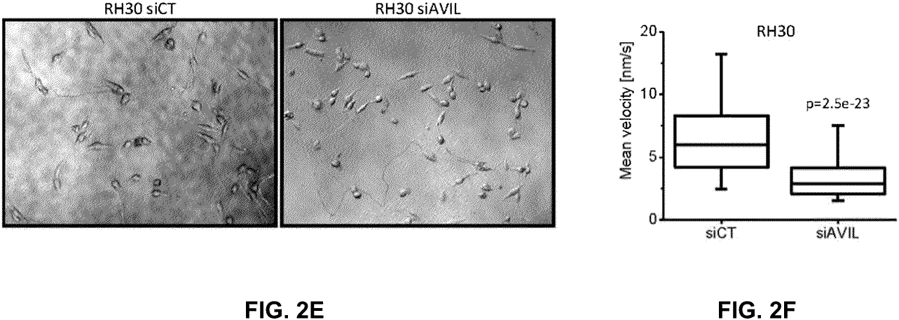

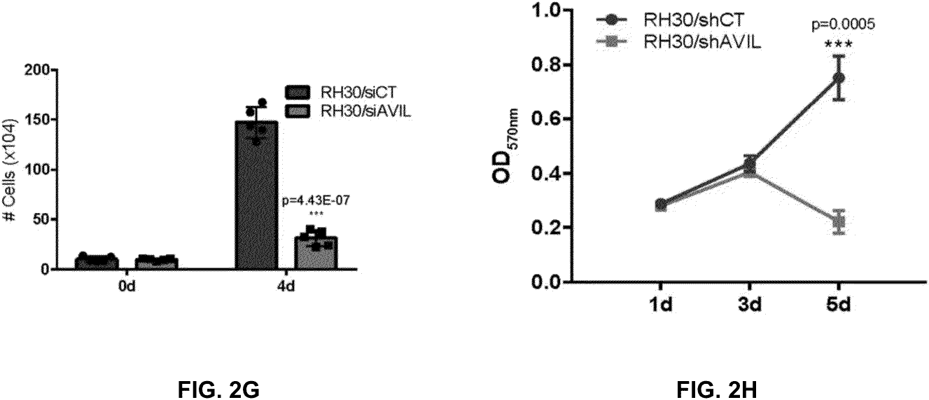

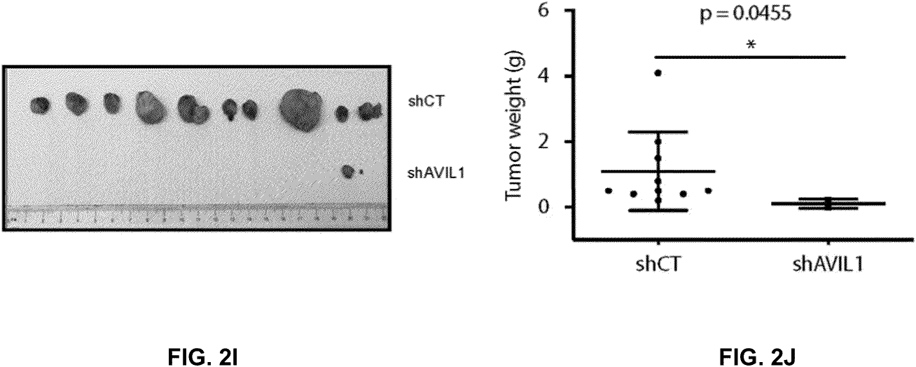

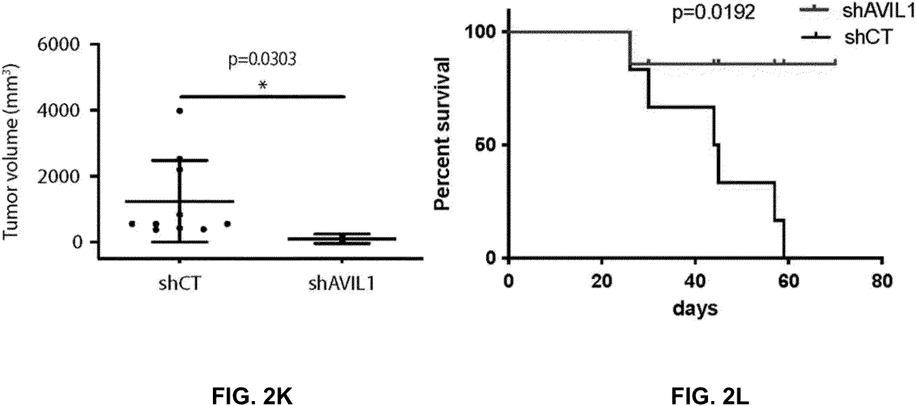

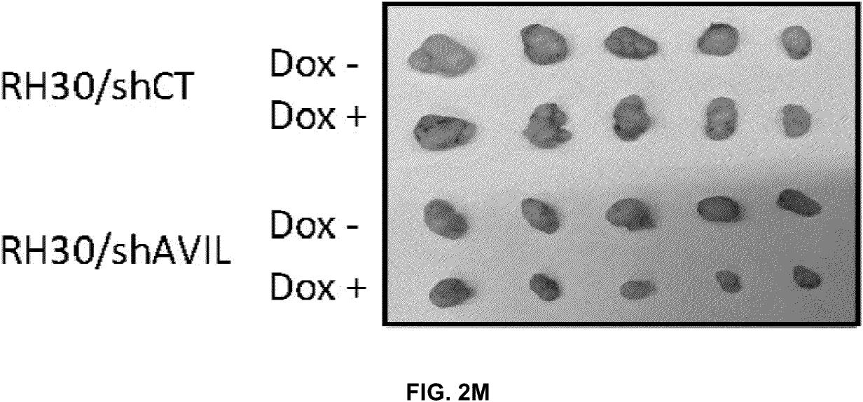

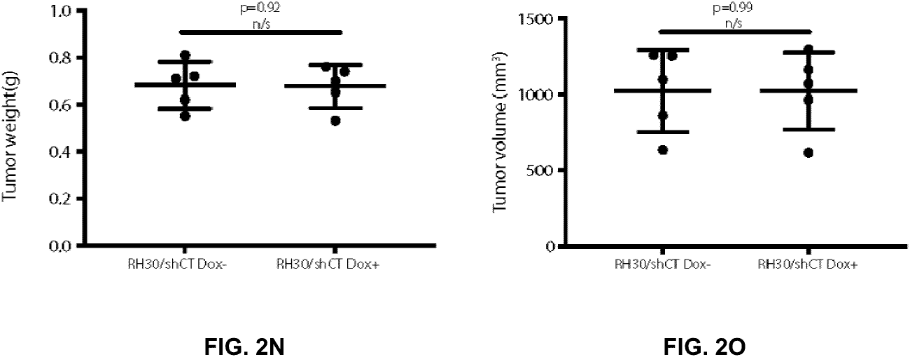

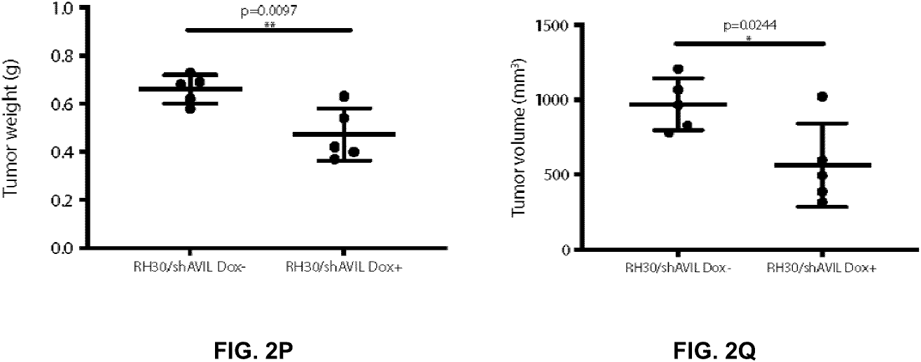

MARS-AVIL is necessary for RH30 tumorigenesis in vitro and in vivo. To further investigate the implications of the MARS-AVIL fusion and complement the above gain-of-function study, loss-of-of-function experiments were conducted. Due to the junction sequence constrain, it was not possible to design a fusion-specific siRNA. To overcome the hurdle, two siRNAs were designed, one targeting both the fusion and wild type AVIL (siAVIL1), the other only targeting wild type AVIL (siAVIL2) (FIG. 9A). When RH30 cells were transfected with siAVIL1, but not siAVIL2, there was a dramatic reduction in cell number, which is reflected by a significant increase in the subG1 peak in cell cycle analysis (FIG. 2A). Consistently, increased cleaved PARP and cleaved Caspase3 signals was observed in cells transfected with siAVIL1, but not with siAVIL2 (FIG. 2B). Most RH30 cells died when transfected with siAVIL1 reflected by crystal violet staining (FIG. 2C). In addition to cell death, early time point after siAVIL1 transfection resulted in slower migration, as demonstrated by a wound-healing assay (FIG. 2D) and tracking of individual cells by live-cell imaging (FIGS. 2E, and 2F). The same effect on cell number was also seen with an shRNA targeting AVIL (FIGS. 2G and 2H). To further rule out off-target effects of siAVIL1 and confirm that the phenotype was due to the silencing of the fusion, but not the wild type AVIL, rescue experiments were performed. Even though not all the cells got transfected, MARS-AVIL, but not the AVIL expression vector, had a partial rescue of cell growth caused by siAVIL1 (FIG. 9B). In an in vivo system, subcutaneous xenografts of RH30 cells either infected with viruses expressing shAVIL1 or control shCT were performed. Injections with shCT infected RH30 cells resulted in tumor formation every time (n=10), whereas injections with shAVIL1 yielded the appearance of only two comparatively smaller tumors (FIG. 2I). Consistently, tumor weight and volume comparisons between the two groups showed dramatic differences (FIGS. 2J and 2K). All of the mice in the control group died or reached the limit for tumor burden and had to be euthanized before 60 days. None of the mice in the shAVIL1 group died of tumor or had a tumor reached the size limit (one mouse was purposely euthanized as a control when the first shCT mice were terminated) (FIG. 2L).

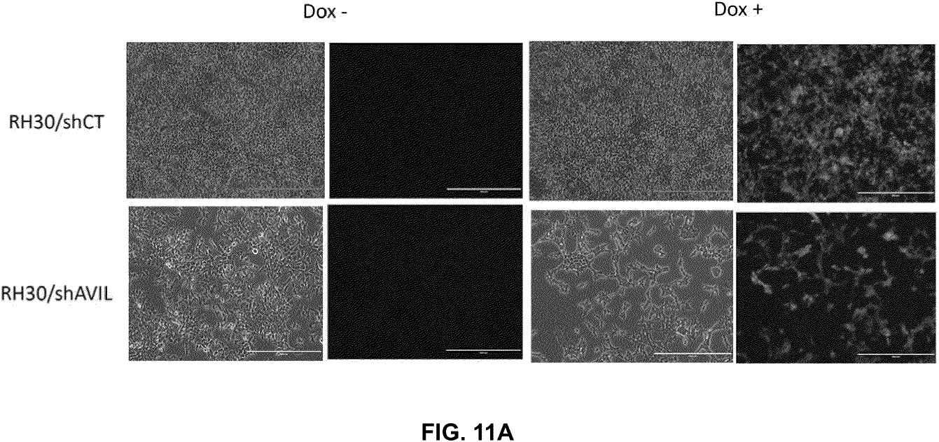

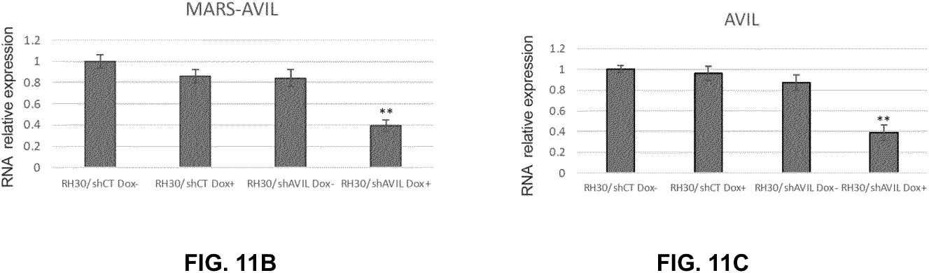

Considering that the shAVIL stably infected cells already proliferated slower than the control cells before injection, tetracycline inducible system was constructed. Three tet-inducible shRNAs against AVIL from transOMIC were acquired. They are in a pZIP-TRE3G backbone, which contains a ZsGreen reporter, and a puromycin resistant gene. Stable cell lines expressing all three shRNAs were then established demonstrating that the GFP expression can be successfully induced by doxycycline (FIG. 11A). Consistently, AVIL was silenced with doxycycline (FIG. 11B). RH30 cells stably expressing one such shAVIL or shCT were then injected subcutaneously into nude mice. The animals were fed with doxycycline-containing or control water. There was significant reduction of tumor weight and volume in shAVIL group only in the presence of doxycycline, but not in the shCT group (FIGS. 2M-2Q).

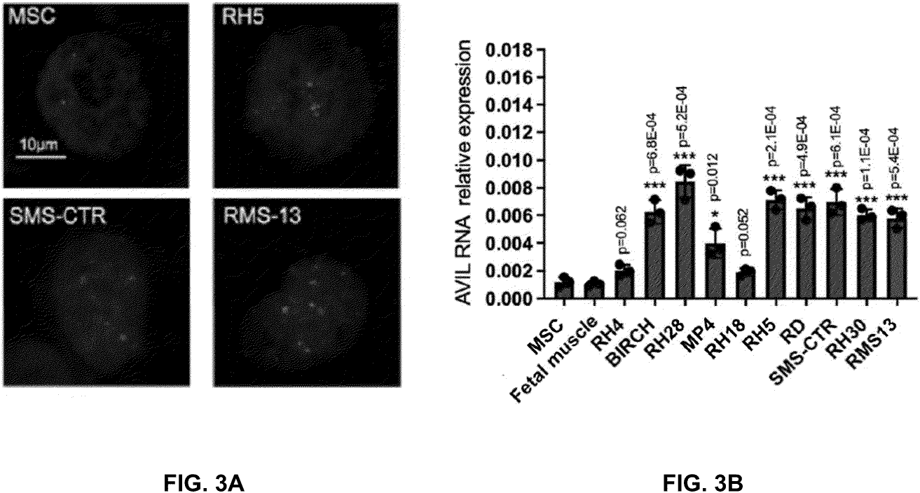

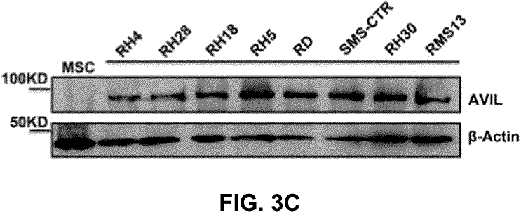

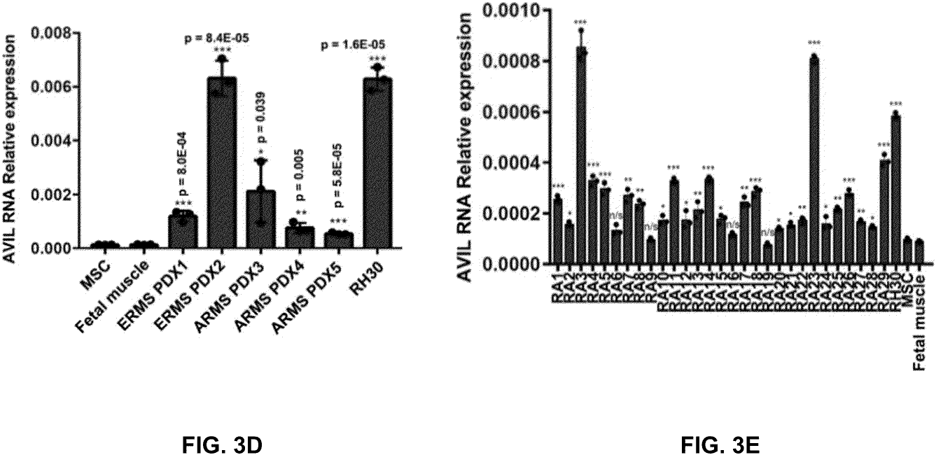

AVIL is overexpressed in other RMS cells. It was speculated that forming the gene fusion with a 5′ house-keeping gene is one way to upregulate AVIL, and AVIL may be dysregulated by other mechanisms. It is known that the 12q13-15 locus is frequently amplified in multiple tumor types, including RMS (Reifenberger, G. et al. Cancer Res 1996 56:5141-5145). Consistently, copy number gain was observed in several RMS lines using AVIL probe in fluorescence in situ hybridization (FISH) (FIG. 3A). Numerous dots in RMS-13 cells indicates that either the fusion or the wild type AVIL, or both are amplified. In addition to the copy number gain, AVIL is overexpressed transcriptionally. AVIL was overexpressed in the majority of RMS cell lines of both embryonal and alveolar histology by quantitative RT-PCR (FIG. 3B). At the protein level, RMS lines regardless of ERMS or ARMS, express a higher level of AVIL than MSC (FIG. 3C).

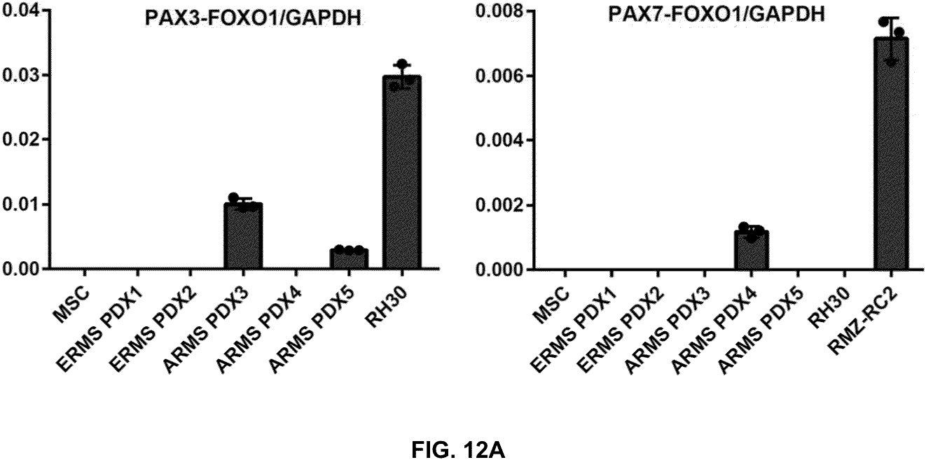

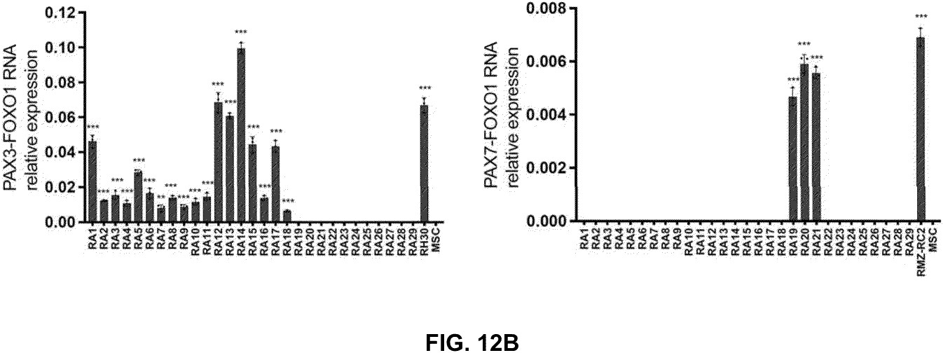

A variety of patient-derived xenograft (PDX) cultures were also obtained from St. Jude Children’s Research Hospital (Stewart, E. et al. Dev Biol 2016 411:287-293), including two PAX3-FOXO1 positives, one PAX7-FOXO1 positive, and two PAX3/7 fusion negative samples (FIG. 12A). All the PDX cells had higher expression of AVIL compared to MSC and a fetal muscle biopsy, measured by qRT-PCR (FIG. 3D). Finally, AVIL overexpression was confirmed in the collection of a clinical cohort, regardless of their PAX3/7 fusion status (FIGS. 3E and 12B).

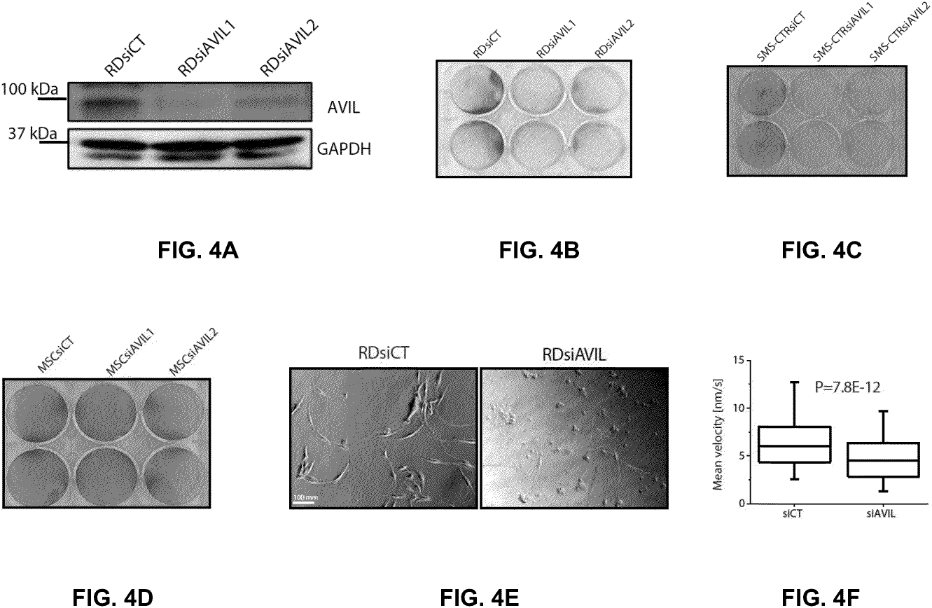

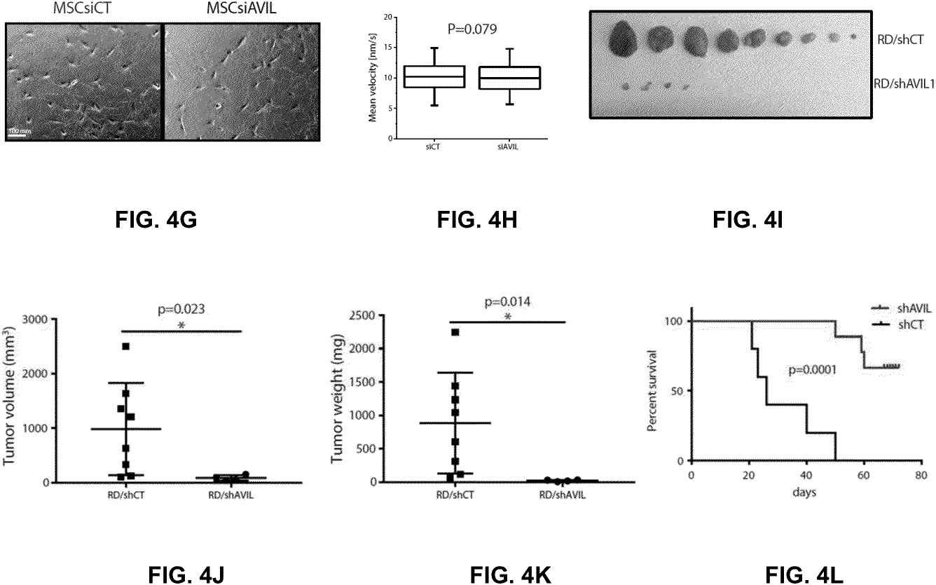

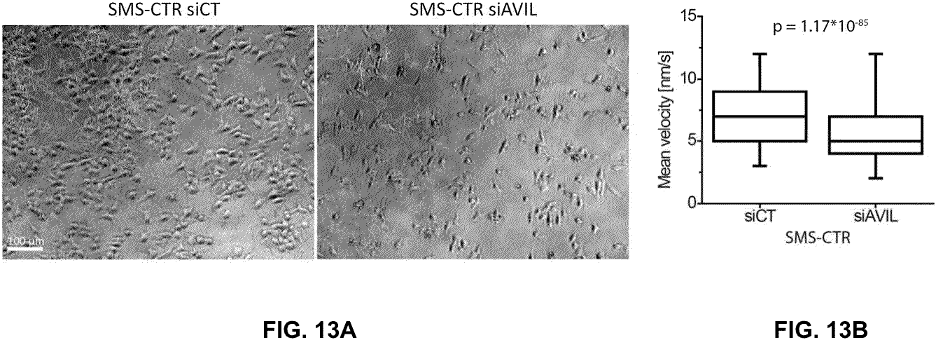

AVIL overexpression is necessary for the tumorigenesis of RMS. Next, we directly tested whether the overexpression of AVIL is required for the tumorigenesis of RMS. In cell lines, RD and SMS-CTR, which express a relatively higher level of AVIL, silencing AVIL with two different siRNAs wiped out the culture (FIGS. 4A-4C). In contrast, no detrimental effect was observed with either siRNAs in MSC cells with low AVIL expression (FIG. 4D). Using live-cell imaging, individual cells were tracked at the early time point after transfection and observed significant reduction with siAVIL in cell motility in RD and SMS-CTR cells (FIGS. 4E, 4F, and 13). In contrast, no significant difference was observed in MSC cells (FIGS. 4G and 4H). These findings support that a high level of AVIL expression is necessary for RMS cell growth and migration. The effect of AVIL silencing in vivo was then tested. Subcutaneous injection of RD cells infected with control shCT produced tumor in nine out of nine experiments. In contrast, injection of RD cells infected with shAVIL only yielded four tiny tumors in nine experiments (FIG. 4I). Significant differences were observed in tumor volume, weight, and animal survival (FIGS. 4J-4L).

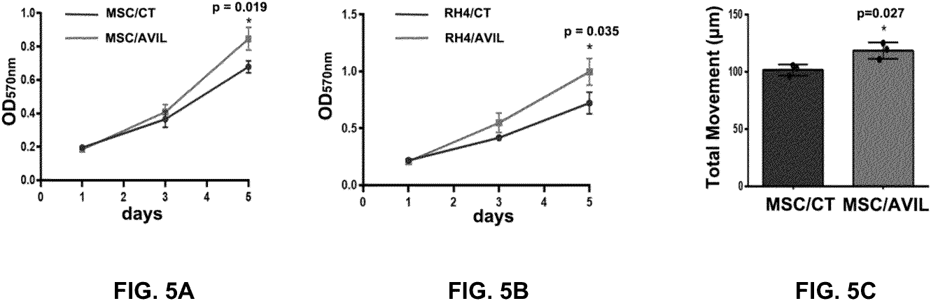

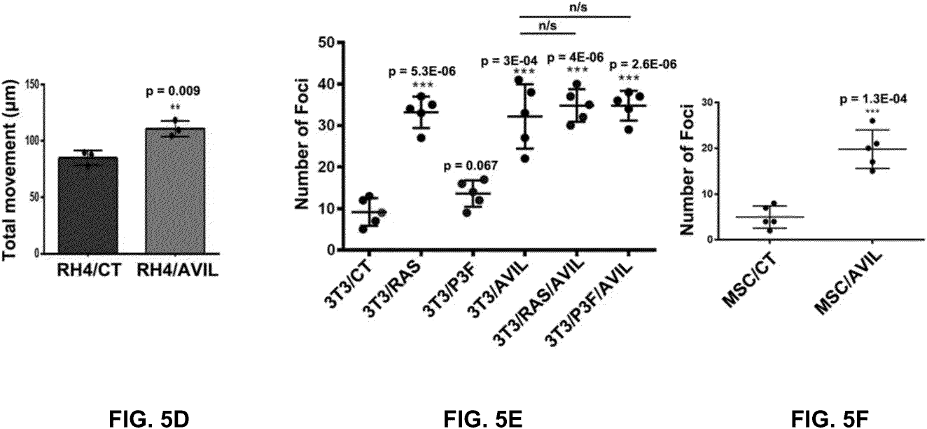

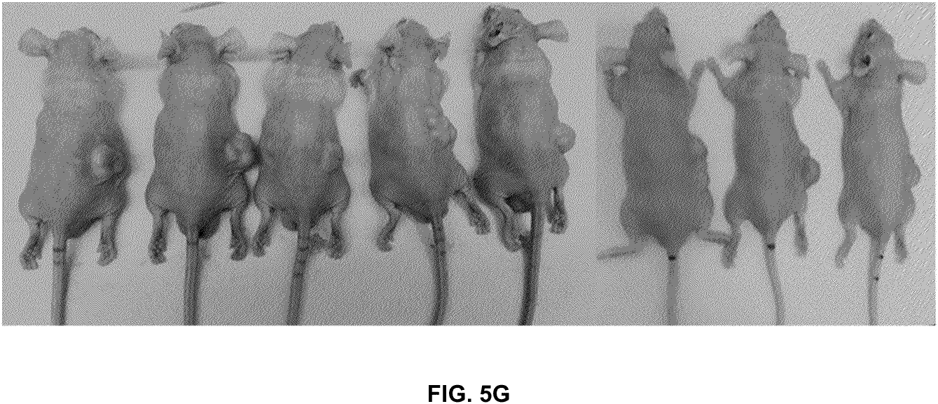

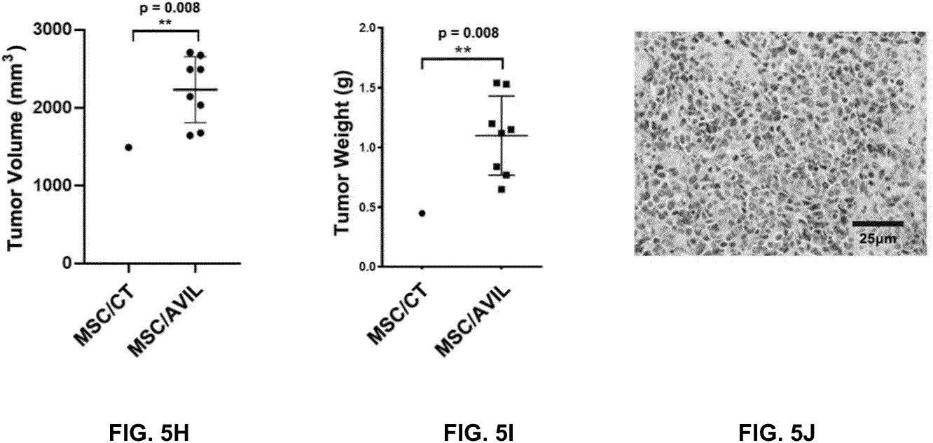

Conversely, in gain-of-function systems, overexpressing AVIL in cell lines including MSC and RH4 resulted in a higher proliferation rate and migration (FIGS. 5A-5D), supporting its role in promoting cell proliferation and migration.

AVIL is a bona fide oncogene in RMS. Finally, to test whether AVIL functions as a bona fide oncogene, the classic oncogene test was performed, the focus assay on NIH3T3 cells (Raptis, L. et al. Methods Mol Biol 2001 165:151-164). There were significantly higher numbers of foci in cells transfected with AVIL than with an empty vector control (FIG. 5E). To examine the potential collaborative effect of AVIL with two major oncogenic pathways (P3F fusion and RAS) in RMS, we performed an oncogene cooperativity assay, where we introduced P3F or RAS alone, or in combination with AVIL overexpression. As shown in FIG. 5E, NIH3T3 cells with RAS transfection resulted in many foci formation, while P3F overexpression did not produce a significantly higher number of foci than empty vector control. Impressively, overexpressing AVIL alone yielded a similar number of foci as RAS. In addition, combining AVIL with the two factors did not result in more foci than AVIL alone, but much more than P3F. These results support that AVIL is sufficient to trigger focus formation and is at least as powerful, if not stronger, than the known oncogenic factors at triggering the escaping of contact inhibition. Consistently, overexpressing AVIL in MSC cells also resulted in a significantly higher number of foci (FIG. 5F).

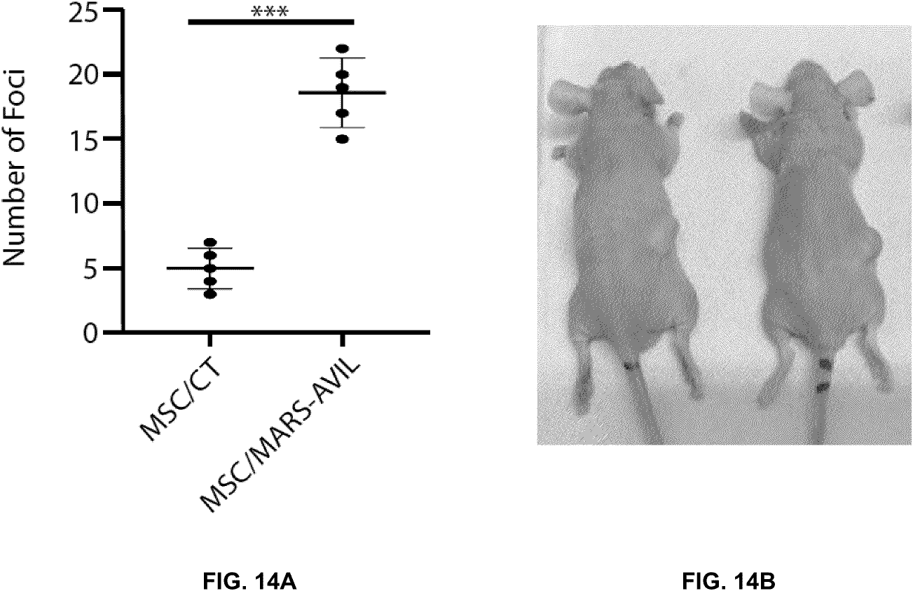

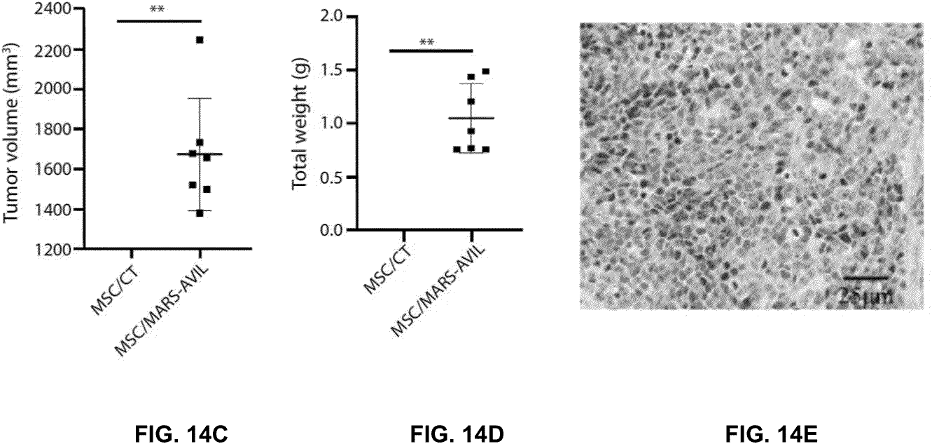

Ultimately, it comes down to in vivo tumorigenesis to test whether a gene is a bona fide oncogene. Here, it was found that overexpressing AVIL alone in MSC cells is sufficient to transform the cells in a subcutaneous xenograft model (FIG. 5G). In the same animals, the left flank was injected with MSC cells transfected with empty vector control (MSC/CT). In contrast, the right side was injected with MSC cells transfected with an AVIL overexpression vector (MSC/AVIL). Eight out of nine animals developed a large mass on the right side, whereas only one animal had a small mass on the left side. Consistently, the tumor volume and weight are significantly different between the MSC/AVIL and MSC/CT (FIGS. 5H and 5I). The masses being actual tumors by histology was then confirmed (FIG. 5J). Similar to AVIL, MARS-AVIL overexpression resulted in more foci formation (FIG. 14A) and can transform MSC cells in vivo (FIGS. 14B-14E).

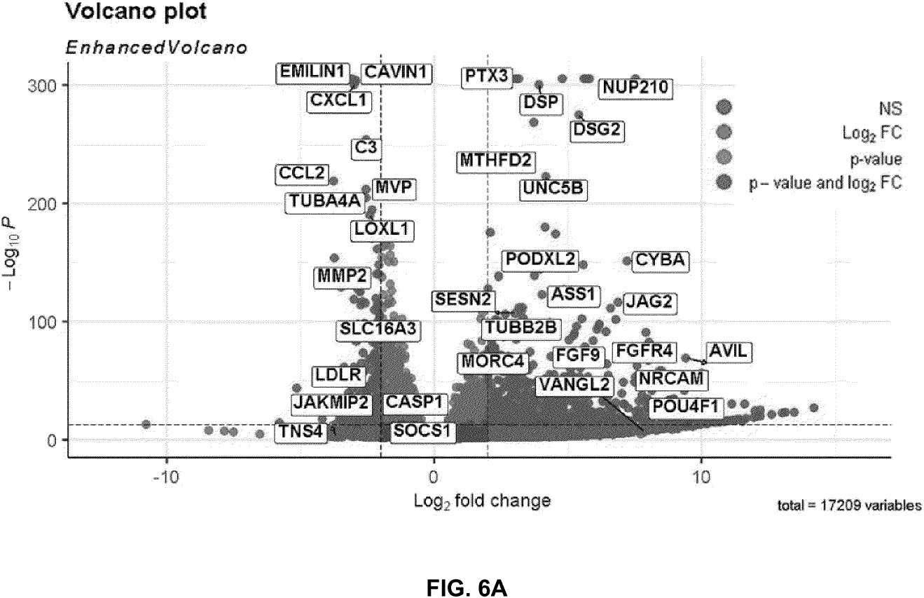

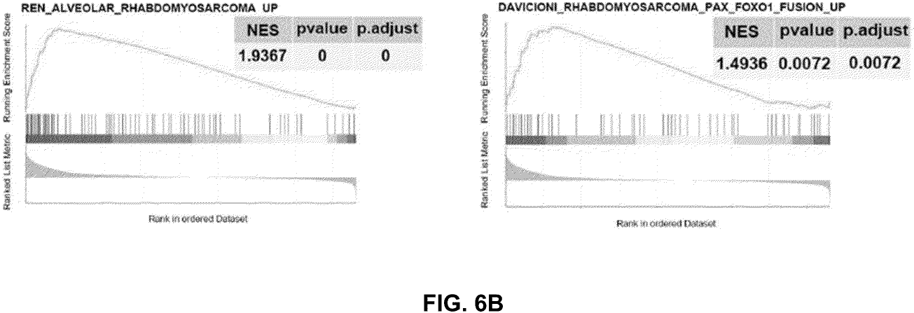

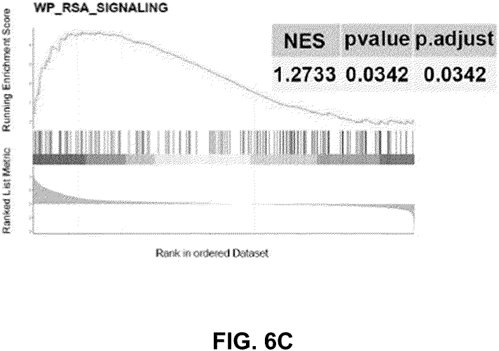

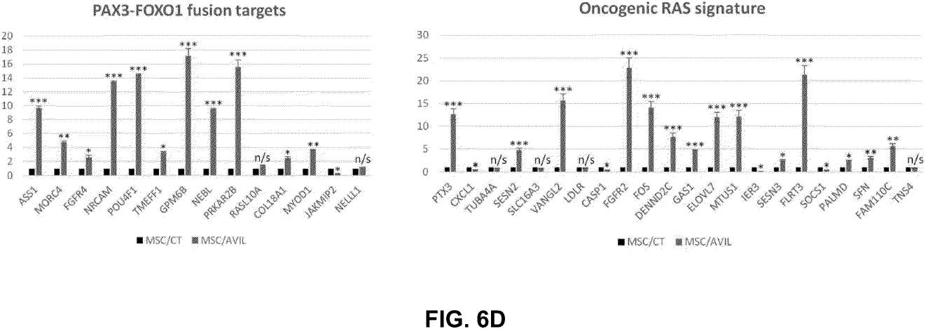

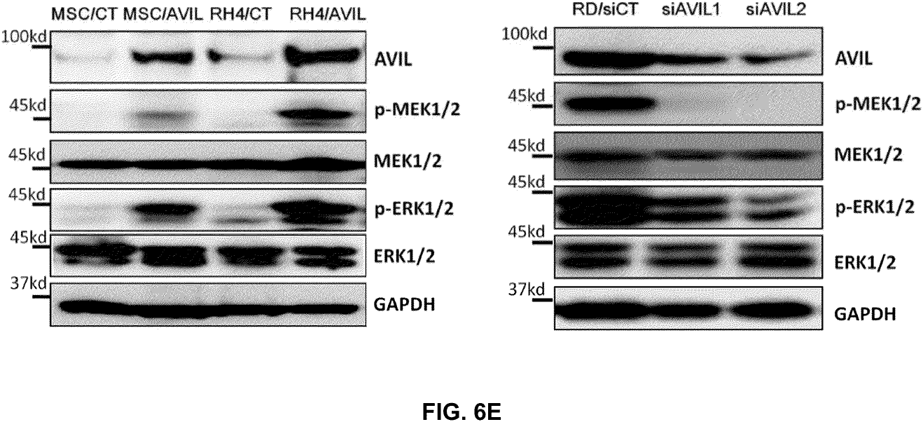

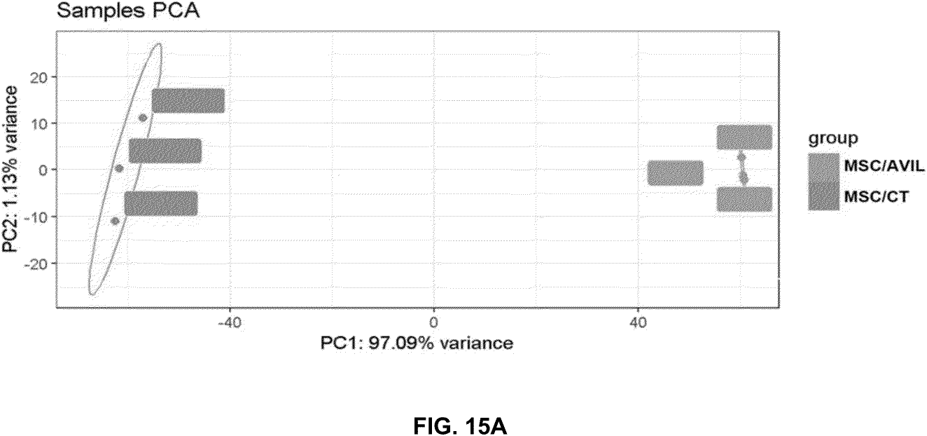

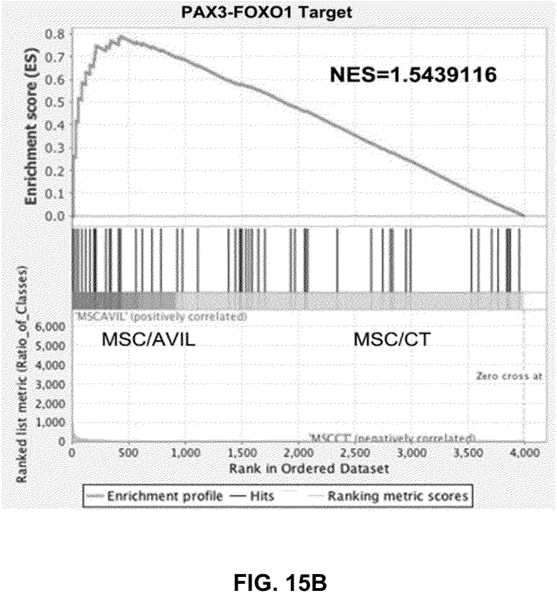

AVIL may be the converging node of two oncogenic pathways. The fact that AVIL is overexpressed in both ARMS and ERMS cell lines as well as clinical samples made us wonder whether AVIL may function as a common node for both types of RMS, merging the two tumor driving pathways. Consistently, the cooperative foci formation assays revealed that addition of P3F or RAS did not yield more foci than AVIL alone. We conducted triplicate RNA-Sequencing experiment comparing the gene expression in AVIL overexpressed MSC cells vs. control MSC cells (FIG. 15A). Many targets of both P3F and RAS were differentially expressed (FIG. 6A). Indeed, GSEA analyses revealed the enrichment of PAX-FOXO1 gene expression signature that defines molecular classes and determines the prognosis of alveolar rhabdomyosarcomas (Davicioni, E. et al. Cancer Res 2006 66:6936-6946), as well as a gene set found in mouse MSC cells expressing PAX-FOXO1 fusion (Ren Y. X. et al, et al. Cancer Res 2008 68:6587-6597) (FIG. 6B). Consistently, these differentially expressed genes were also over-represented in the 76 genes recently reported as PAX3-FOXO1 targets using ChlP-seq (Cao L. et al. Cancer Res 2010 70:6497-6508) (FIG. 15B). Interestingly, a curated RAS gene signature was also enriched (FIG. 6C). qPCR was performed to measure 14 known downstream targets of PAX3 and PAX3-FOXO1 including MYOD1 and FGFR4. 12 were found to be significantly changed upon AVIL overexpression (FIG. 6D). Similarly, 22 RAS downstream target genes were selected for validation by qRT-PCR, and 18 confirmed being induced or suppressed with AVIL significantly. To further conform RAS pathway activation, we measured protein level changes of phosphorylated MEK1/2, and ERK1/2. In both MSC and RH4 systems, overexpressing AVIL enhanced the level of phosphorylated MEK1/2, and ERK1/2 (FIG. 6E). Conversely, silencing AVIL in RD cells resulted reduced signal of both phosphorylated MEK1/2, and ERK1/2.

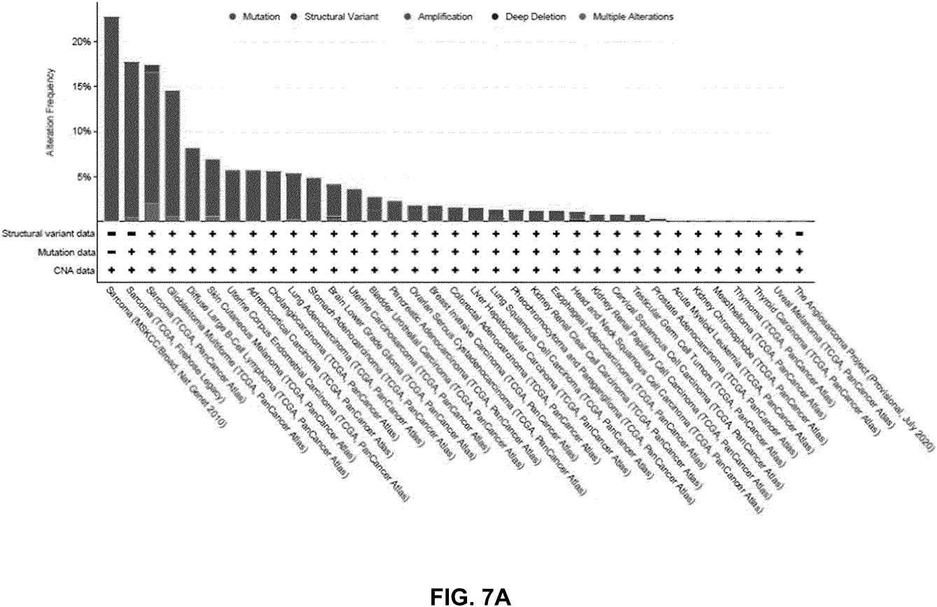

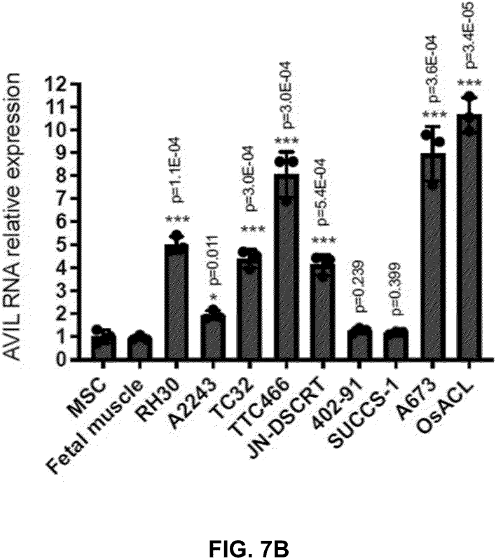

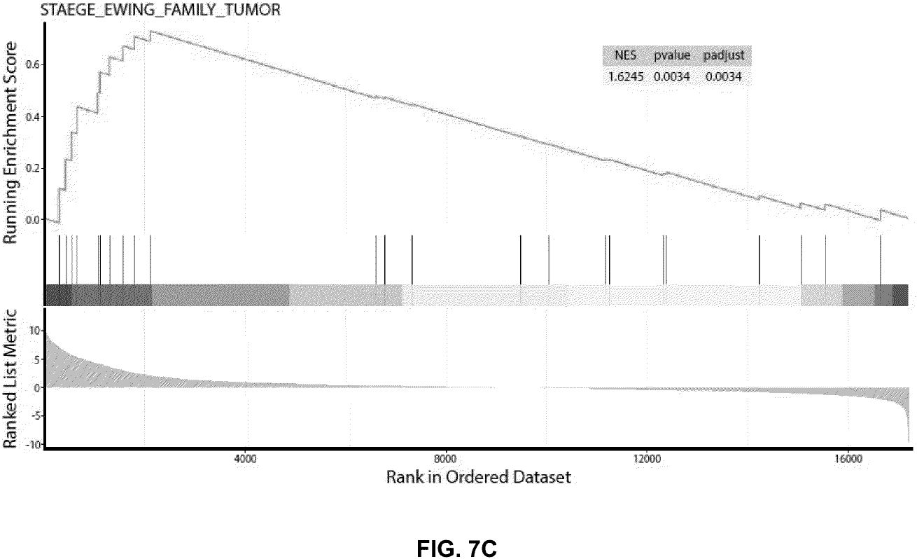

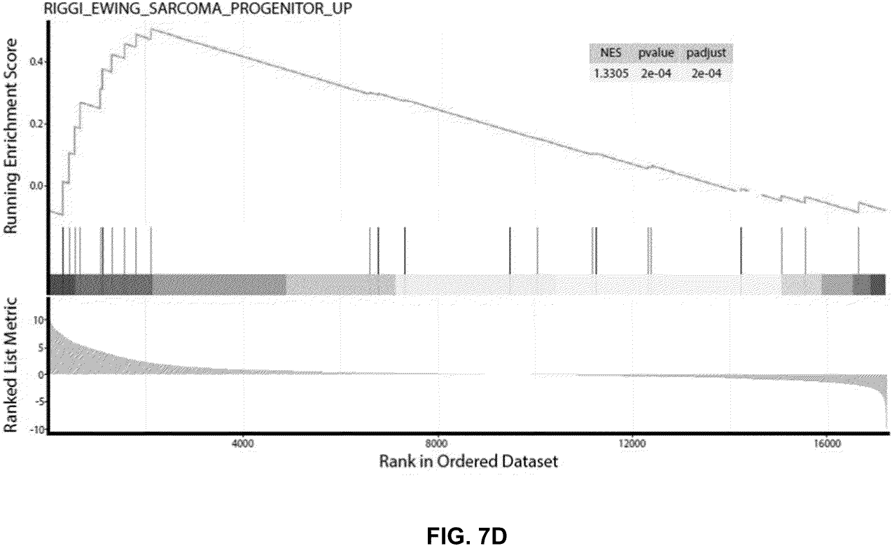

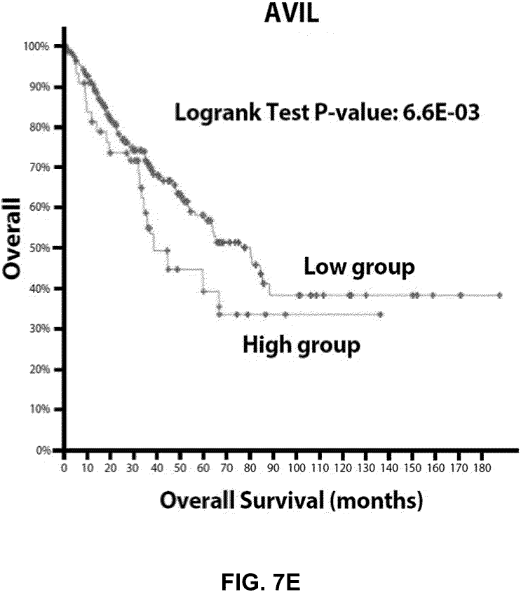

AVIL activation may be a general oncogenic pathway in sarcomas. The AVIL locus was frequently amplified in sarcoma (FIG. 7A). By qPCR, it was found that A VIL RNA was also overexpressed in many other sarcoma cell lines, including Ewing sarcoma, myxoid liposarcoma, desmoplastic small round cell sarcoma, clear cell sarcoma, osteosarcoma, and synovial sarcoma cell lines (FIG. 7B). Consistently, there was enrichment of Ewing family tumor signature (Staege, M. S. et al. Cancer Res 2004 64:8213-8221) as well as EWS-FLI triggered Ewing sarcoma progenitor signature (Riggi N. et al. Cancer Res 2008 68:2176-2185) (FIGS. 7C and 7D). These findings stimulated us to examine the expression of AVIL in the TCGA sarcoma database for patient survival. Indeed, a higher level of AVIL expression is correlated with significantly worse clinical outcomes than the AVIL low group (FIG. 7E).

### Discussion

A potent oncogene might use multiple mechanisms to misregulate its activity, which at the same time give credence to the gene being a critical player in tumorigenesis and malignancy. AVIL forms a fusion with a housekeeping gene MARS in the patient where RH30 and RMS13 were established. Its locus is amplified by copy number in another subset of RMSs. The rest majority likely use transcriptional and/or post-transcriptional mechanisms to upregulate its expression at RNA and protein level. In addition to RMS, AVIL was overexpressed in many other sarcoma types including, Ewing, synovial, osteosarcoma and liposarcoma. These and our previous finding that AVIL plays a critical role in glioblastoma (GBM) (Xie Z. et al. Nat Commun 2020 11:3457; Xie, Z. et al. Mol Cell Oncol 2020 7:1804309; Cornelison, R. et al. Int J Mol Sci 2021 22) suggest that AVIL may be a general oncogene, not limited to a particular cancer type.

AVIL is overexpressed in RMS cell lines, PDX, and clinical samples at RNA and protein levels regardless of their molecular categories of PAX3/7-FOXO1 fusion-positive and fusion-negative. Intriguingly, in the NIH3T3 oncogene cooperativity assay, the combination of RAS or PAX3-FOXO1 with AVIL did not result in more foci than AVIL alone, suggesting that AVIL may be a node connecting oncogenic pathways for both categories of RMS. Supporting this hypothesis, the RAS pathway and PAX3-FOXO1 targets are both enriched with Gene Set Enrichment Analysis in MSC cells overexpressing AVIL. Both pathways are also confirmed to be activated upon AVIL overexpression based on qPCR and Western analyses, indicating that AVIL may lie in some common signaling axis in both types of RMS. Besides AVIL, sets of genes altered in fusion negative RMS including MYOD and FGRF4 have been noticed to be also targets of PAX3, and PAX3-FOXO1 (Shern J. F. et al. Cancer Discov 2014 4:216-231), connecting the two pathways.

Alteration of receptor tyrosine kinase/RAS/phosphoinositide 3-kinase (Pl3K) axis affected 93% of RMS cases and that appeared to hinge on the FGF and IGF receptor pathways (Shern J. F. et al. Cancer Discov 2014 4:216-231; Zhu, B. et al. Br J Cancer 2015 112:227-231). It is known that activation of Ras and Rac plays a critical role in mediating downstream motility events, such as membrane ruffling and cell protrusion (Malliri A. et al. J Cell Biol 1998 143:1087-1099). Advillin encoded by AVIL regulates F-actin dynamics. Even though it is less studied, the same family member gelsolin has been shown to be a downstream effector of Rac, but as the same time affect Rac expression (Azuma, T. et al. EMBO J 1998 17:1362-1370). Also gelsolin is evidently a downstream effector of the Ras-PI3K signaling pathway in cellular invasion (De Corte, V. et al. EMBO J 2002 21:6781-6790). Likely, AVIL may reside on the feedback loop of Ras/Rac-PI3K pathways affecting both categories of RMS.

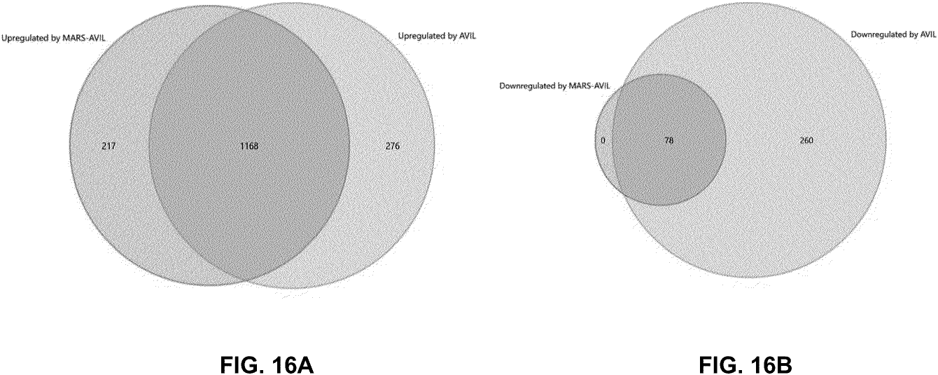

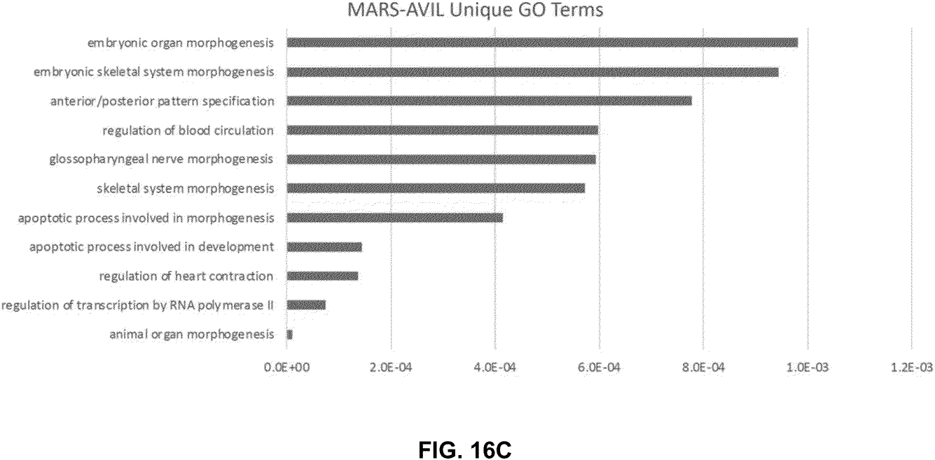

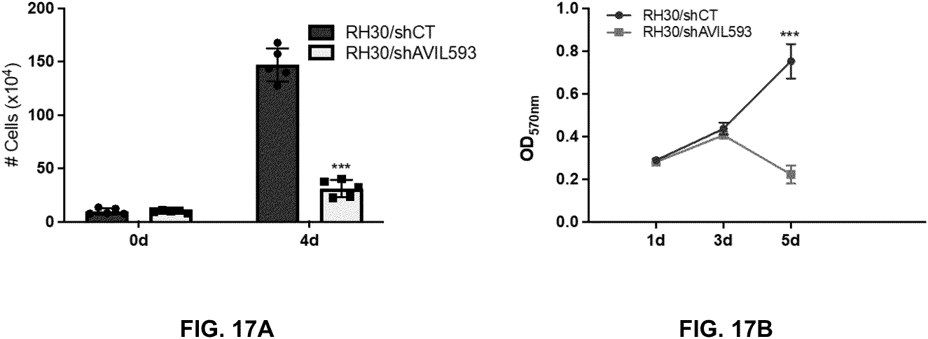

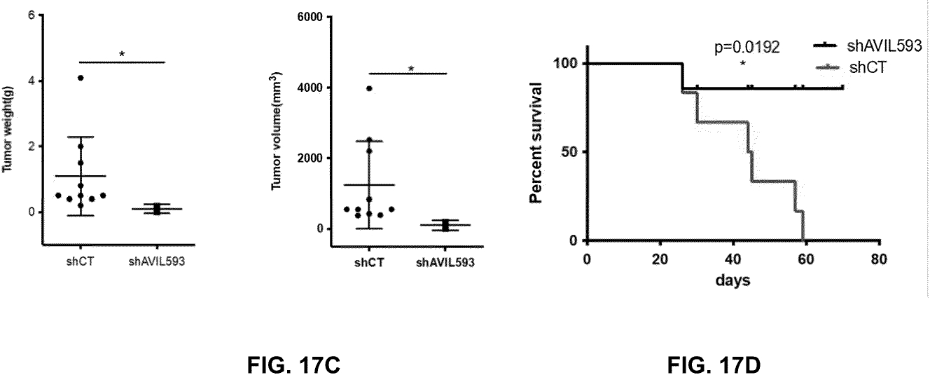

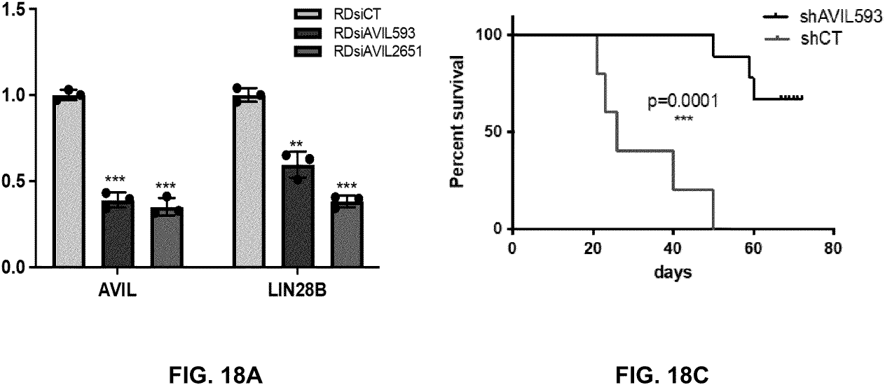

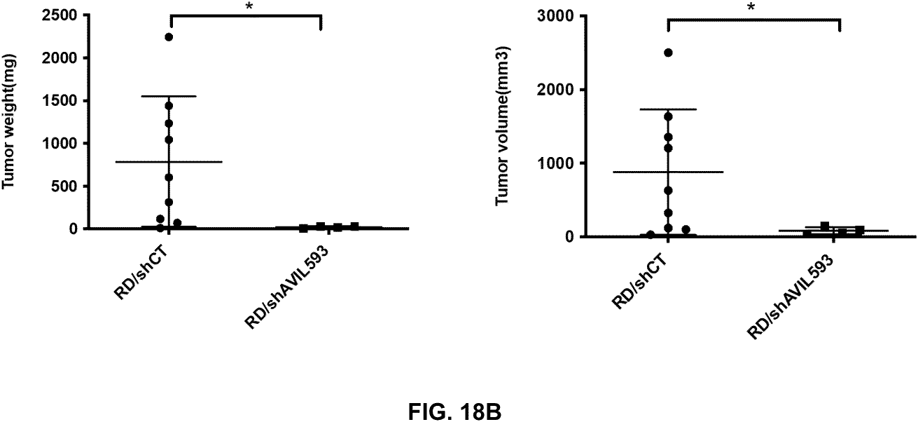

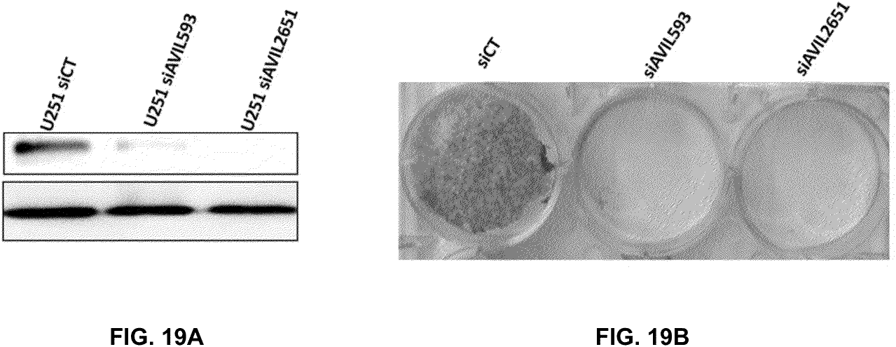

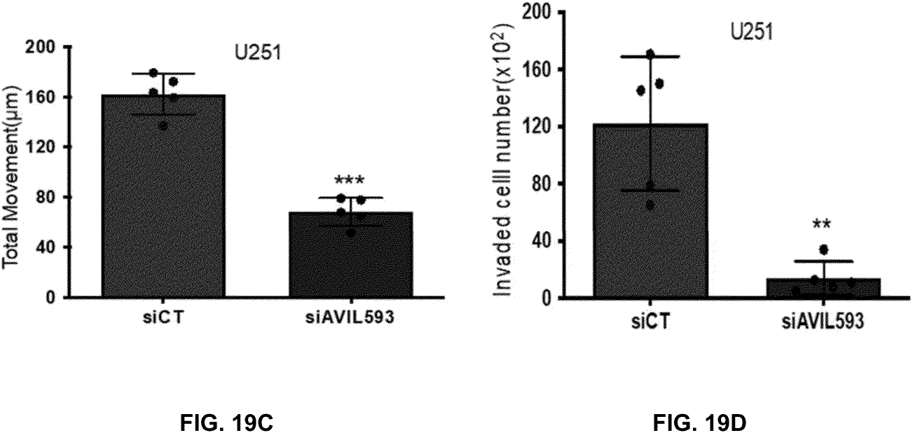

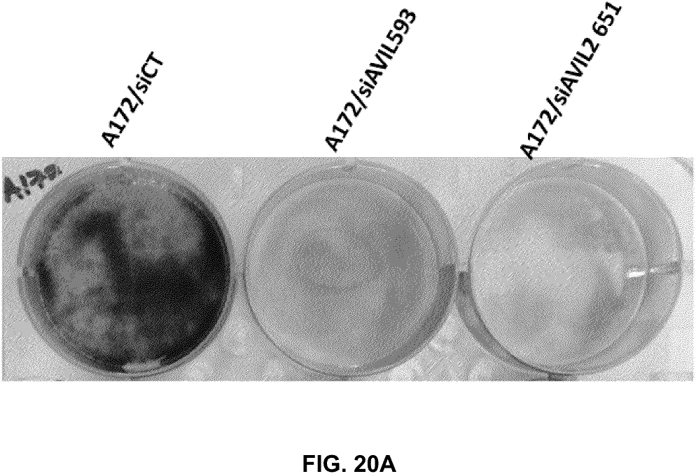

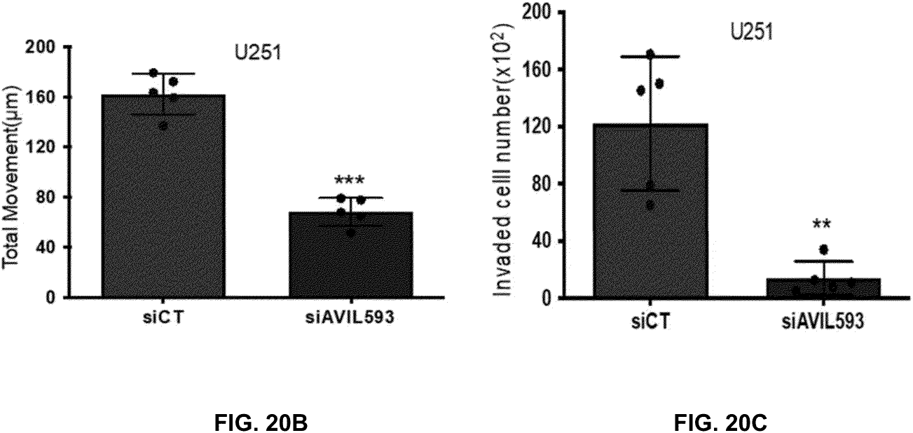

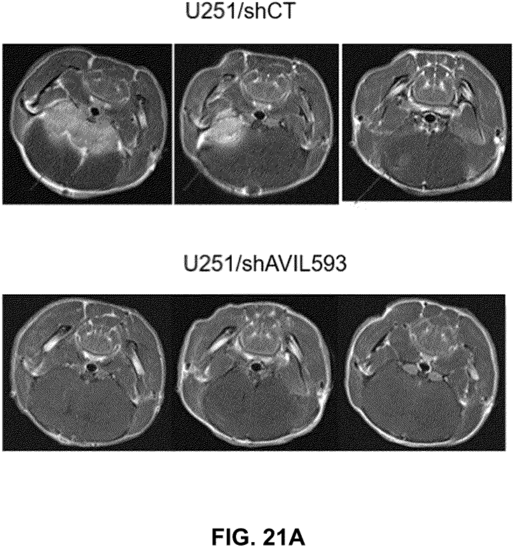

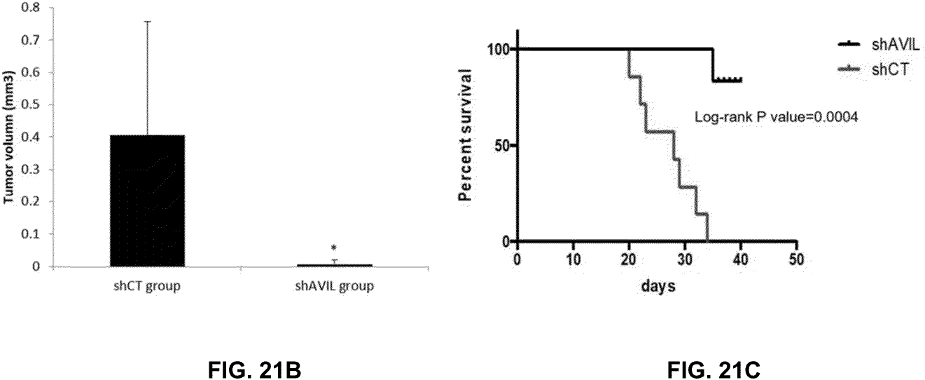

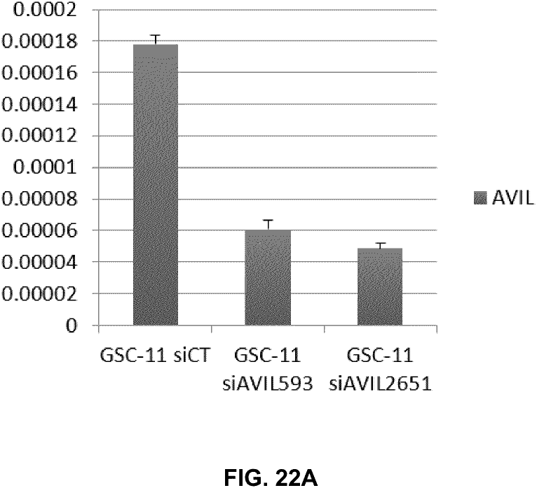

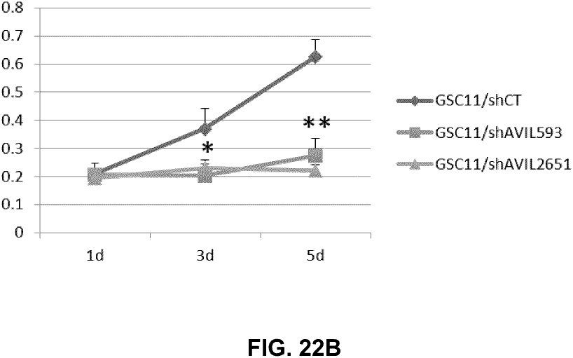

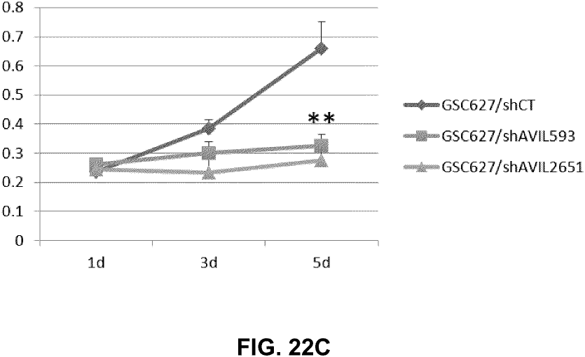

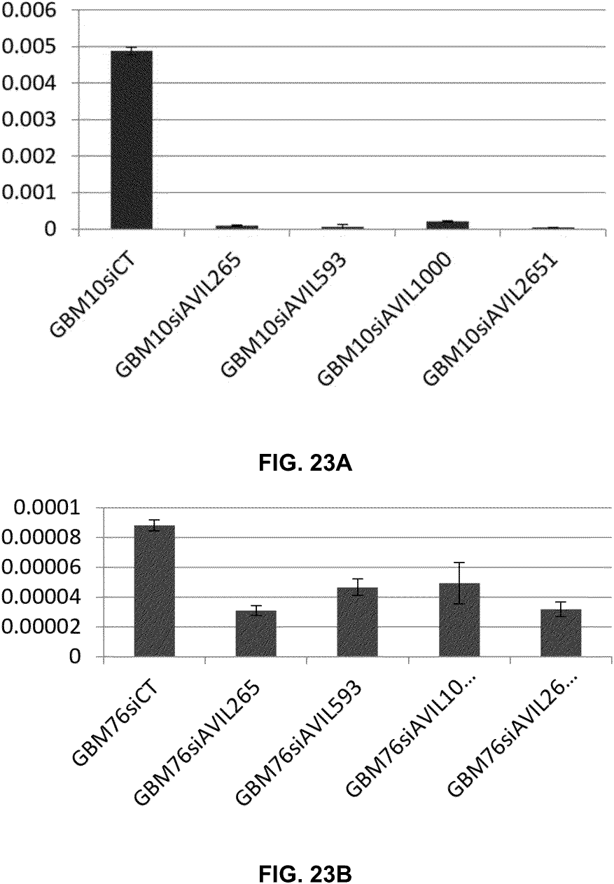

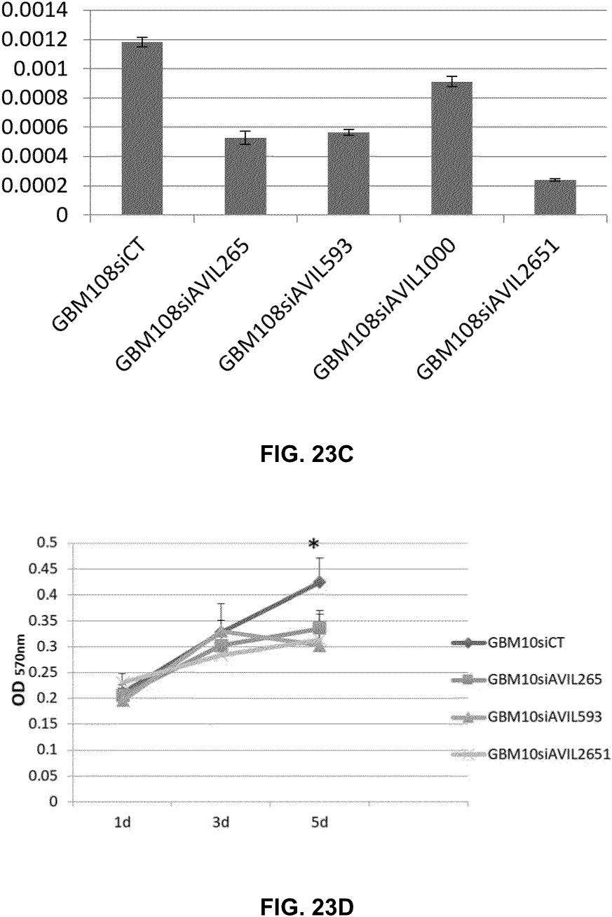

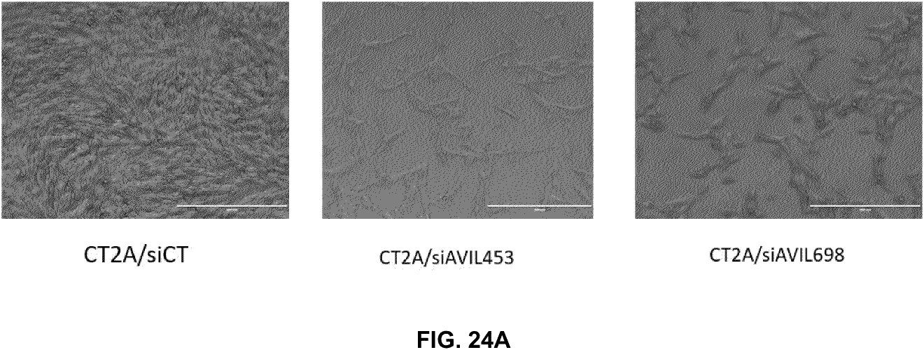

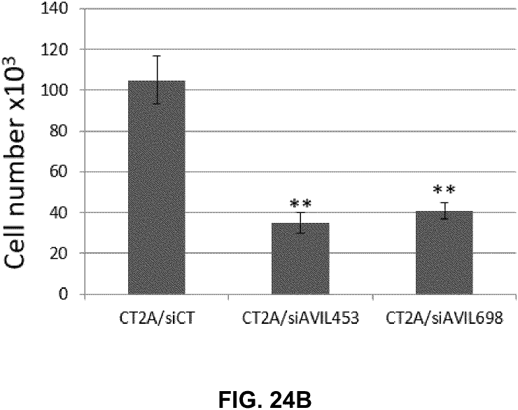

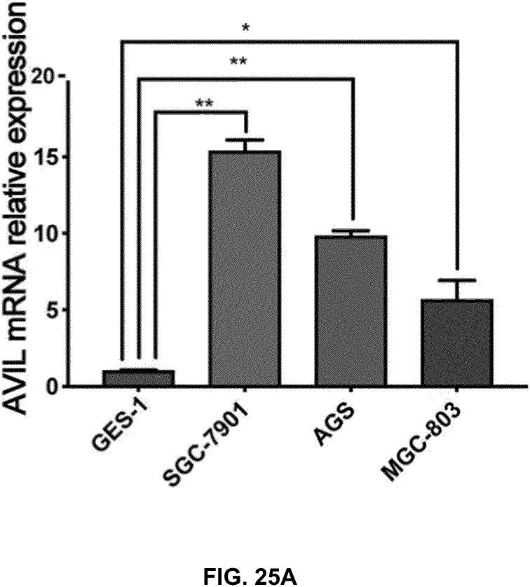

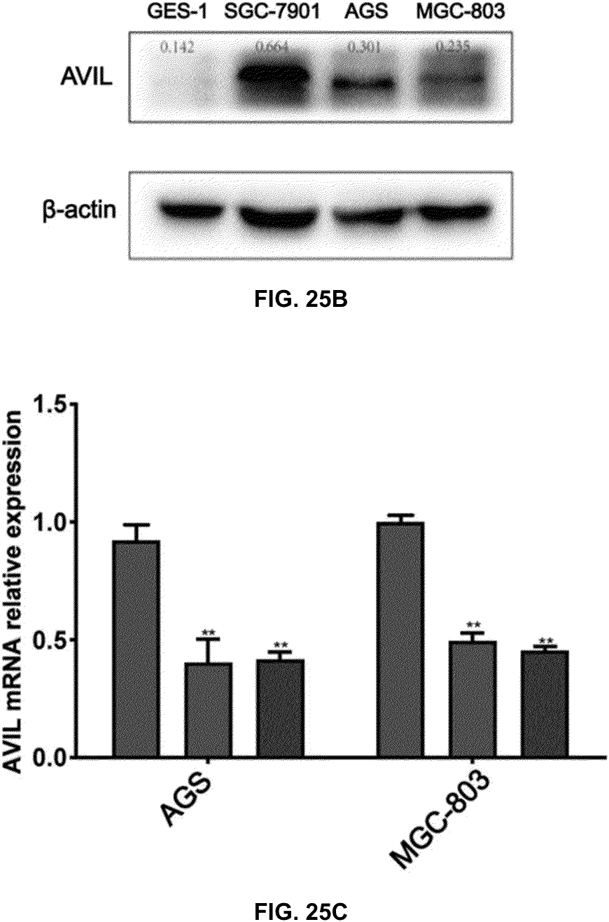

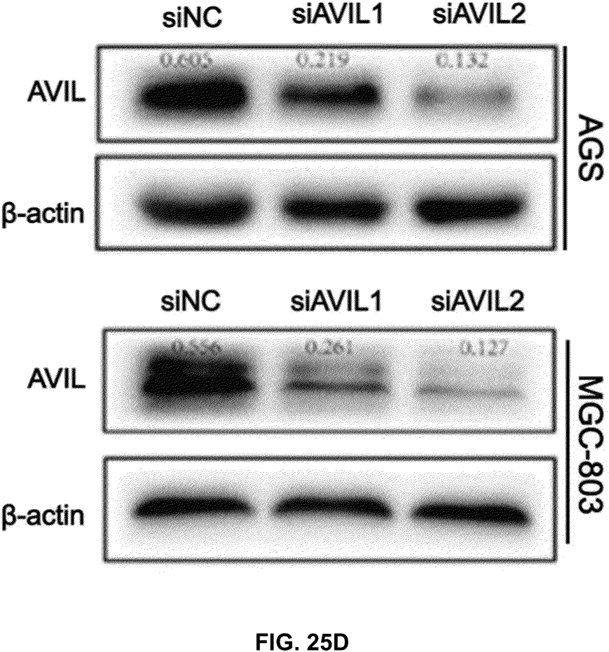

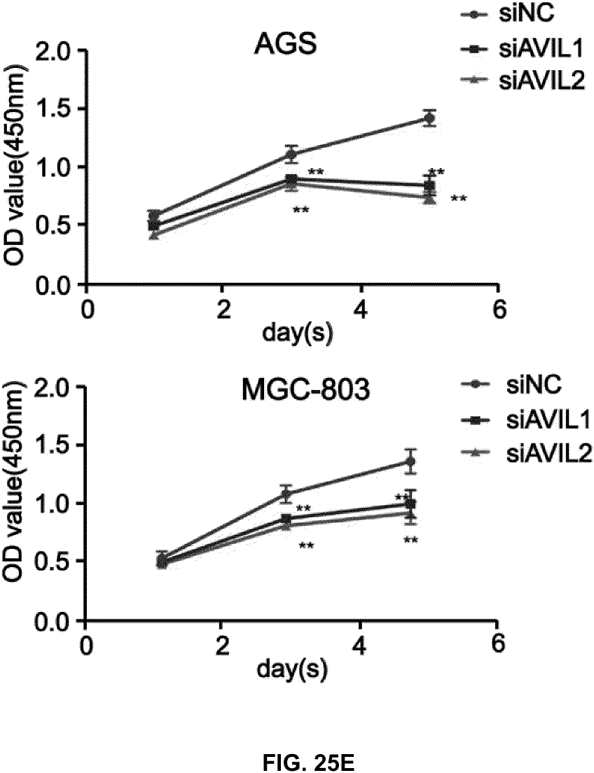

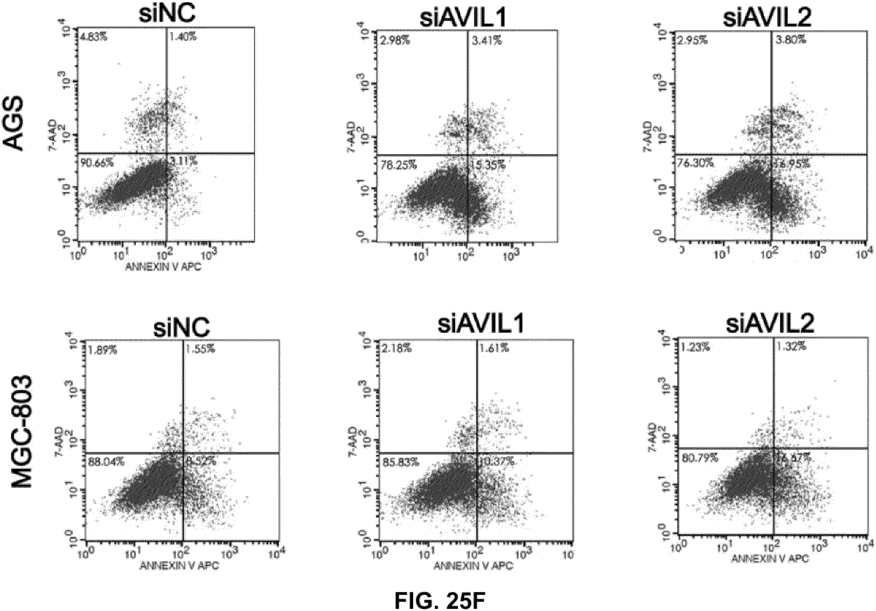

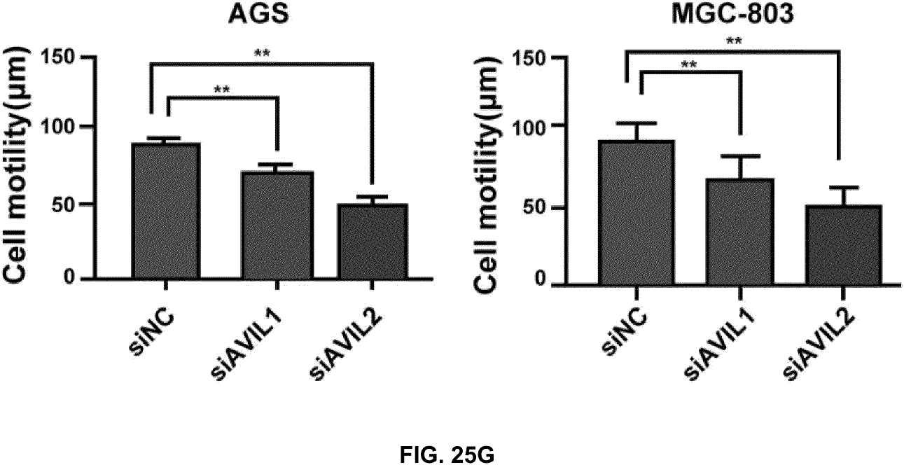

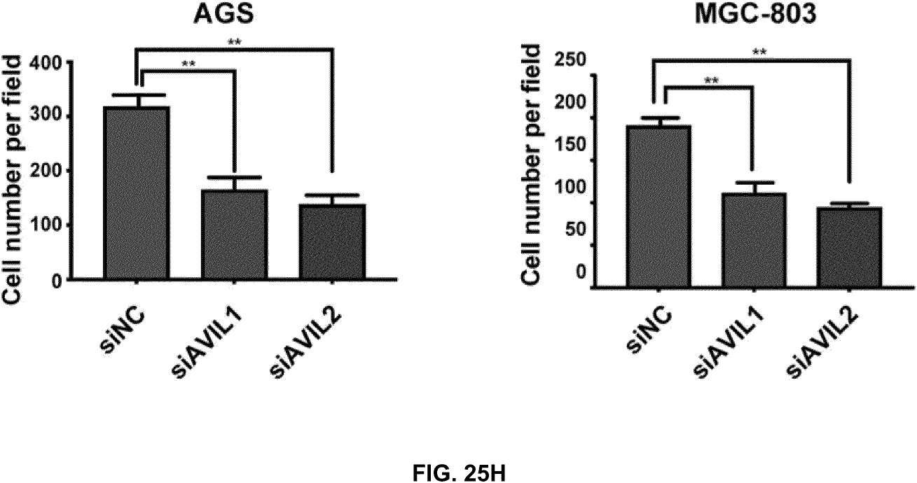

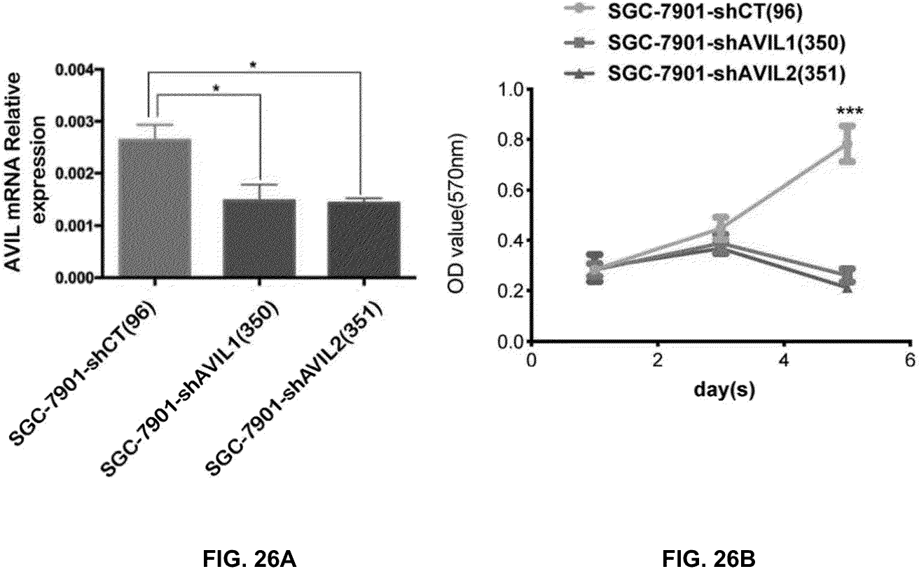

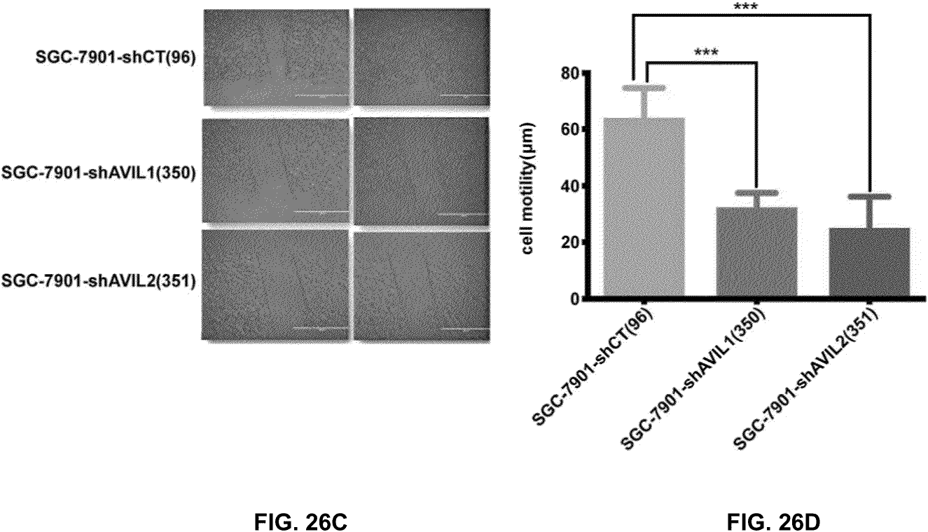

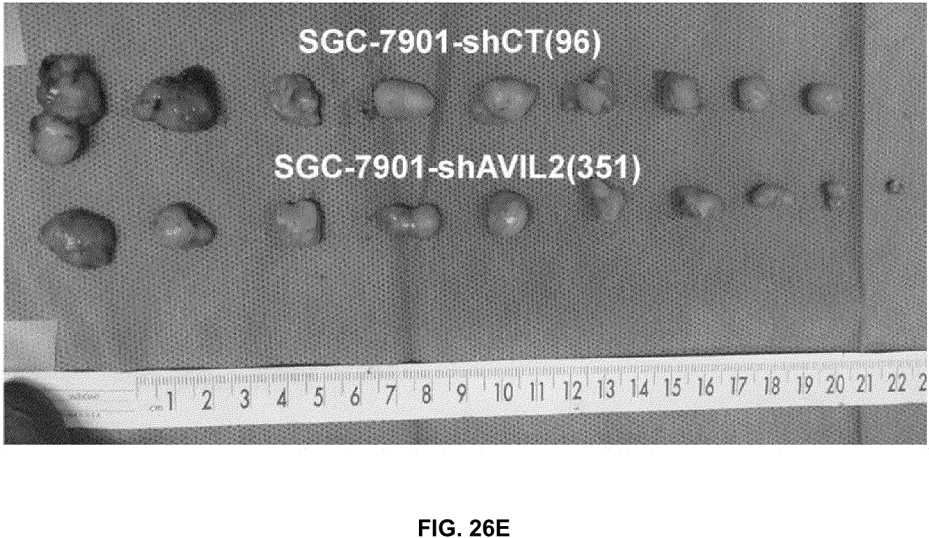

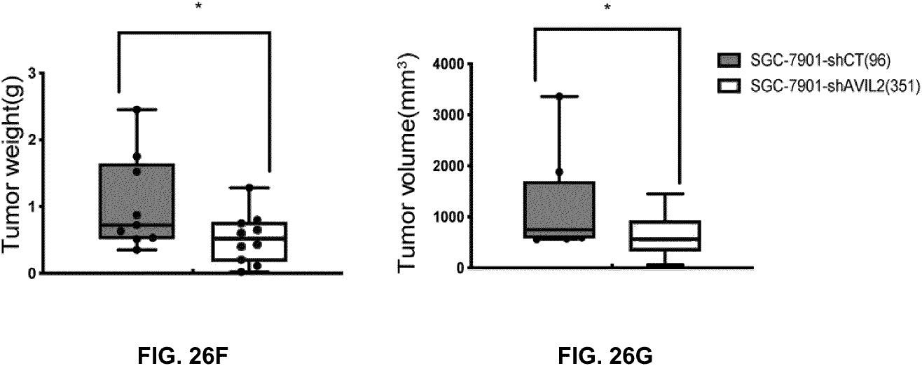

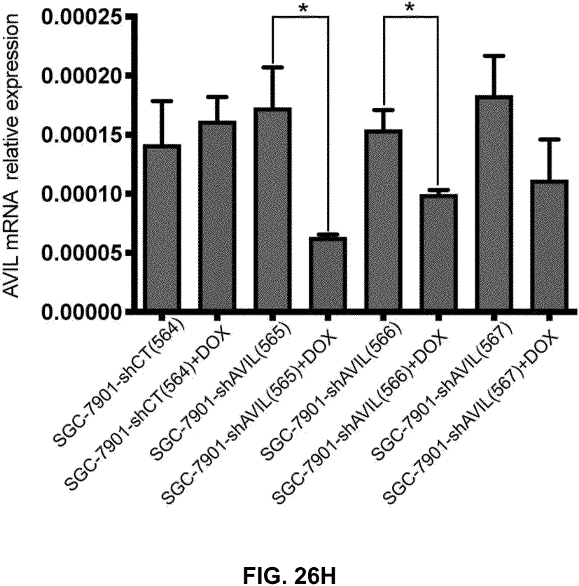

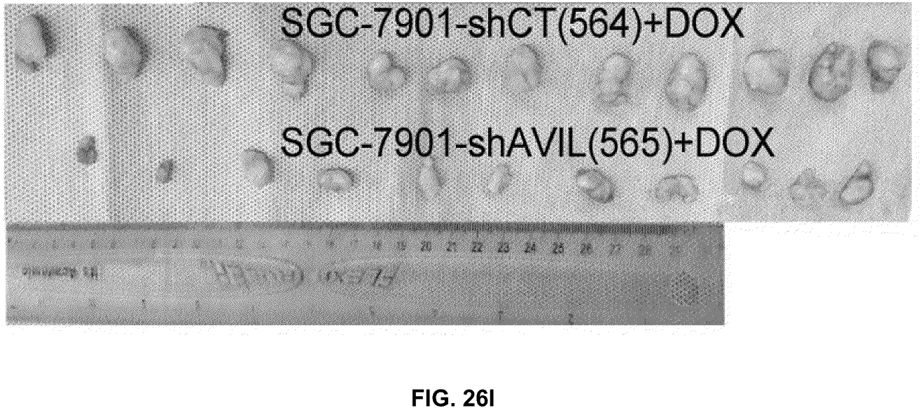

MARS-AVIL fusion is formed by chromosomal inversion and encodes an in-frame fusion protein. Together with PAX3-FOXO1, they are the few shared fusions in RH30 and RMS13, believed to be established from the same donor patient (Hinson, A. R. et al. Front Oncol 2013 3:183). The identical fusion junction sequence is seen in an Image clone deposited in the human non-reference RNA database, with the source of “rhabdomyosarcoma”, suggesting that the fusion is likely to be present in a subset of RMS. However, in collection of 29 RMS clinical samples, we did not detect this fusion, suggesting that it being a rare event. Phenotypically, like wild-type AVIL, MARS-AVIL transfection enhanced cell proliferation, migration, foci formation and enabled MSC transformation in vivo. However, it likely has its unique function as wild-type AVIL cannot rescue the effect caused by MARS-AVIL silencing. Additionally, RNA-Seq of MSC cells expressing MARS-AVIL revealed distinctive profiles from that of MSC cells overexpressing AVIL, in that MARS-AVIL uniquely upregulated about 217 genes, whereas AVIL induced the expression of 276 unique genes. A number of gene ontology terms such as embryonic organ and skeletal system morphogenesis are enriched in MARS-AVIL regulated genes (FIG. 16).

### Methods

### Cell Lines and Culture Conditions

MSCs were purchased from ATCC and were maintained in MEM alpha medium with 20% FBS. The ARMS cell line RH30 was obtained from the laboratory of Anindya Dutta, University of Virginia, Charlottesville, VA. The cells were cultured in RPMI 1640 medium with 10% (vol/vol) FBS. 402-91, A2243, JN-DSRCT, RH4, BIRCH, OsACL, RH28, MP4, RH18, RD, RMS-13, SUCCS-1, TC32, TTC-466 cells were obtained; and A673 cells were purchased from ATCC.

### Clinical and PDX Samples

RNA of Rhabdosarcoma patients were obtained from Dr. Barr. Cryo-preserved cells for implantation and flash frozen PDX samples were gifts from St. Jude Children’s Research Hospital (Stewart, E. et al. Dev Biol 2016 411:287-293).

### AVIL Overexpression and Silencing

MARS-AVIL coding region was RT-PCR amplified from RH30. AVIL cDNA clone was purchased from GeneCopoeia (GC-OG11537), and cloned into the Retrovirus vector pQCXIN. Stable cells that overexpress AVIL were selected via G418. For siRNA treatments, siAVIL1 (targeting 5′-GCTTCTGGCAAAGGATATT-3′ (SEQ ID NO:1)), siAVIL2 (targeting 5′-CTTCAGGGCTGTGGACAAC-3′ (SEQ ID NO:2)), and control siRNA (siGL2) were purchased from Life Technologies. RNAiMAX (Invitrogen) was used for siRNA transfection, which was performed according to the manufacturer’s instructions and previous publication (Wu, P., et al. EBioMedicine 2018 37:158-167). Tet-inducible shAVIL constructs were purchased from transOMIC. Stable cells were selected with puromycin. Cells are treated with doxycycline hydrochloride (final concentration 10 µg/ml).

### PCR and Real-Time PCR

RNA was extracted using RNeasy Mini Kit (QIAGEN) and quantified with Nanodrop (Thermo). cDNA was generated by Verso cDNA Synthesis Kit (Thermofisher), and a random hexamer primer (Xie, Z., et al. RNA Biol 2019 16:144-153). Real-time qPCR was carried out on the StepOne Plus system from Applied Biosystems using SYBR mix (Thermo) according to previous publications (Qin, F., et al. PLoS One 2016 11:e0150382; Zhu, D., et al. Int J Biochem Cell Biol 2019 110:50-58).

### Western Blotting

Cell lysates were resolved by denaturing gel electrophoresis, before performing Trans-Blot Turbo Transfer System (BIO-RAD). The membrane was subjected to western blot analysis with antibodies against the proteins of interest. The following antibodies and dilutions were used: rabbit anti-MARS (1:1000 Sigma SAB2101437), rabbit anti-AVIL (1:1000; Abcam ab72210), rabbit anti-PARP (1:1000; Cell Signaling Technology 9542), rabbit anti-Cleaved Caspase-3 (1:1000; Cell Signaling Technology 9664), MEK1/2 (L38C12) Mouse mAb (1:1000; Cell Signaling Technology 4694S), Phospho-MEK1/2(Ser217/221) Antibody (1:1000; Cell Signaling Technology 9121S), p44/42 MAPK (Erk1/2) Antibody(1:1000; Cell Signaling Technology 9102S), Phospho-p44/42 MAPK(ERK1/2) Antibody (1:1000; Cell Signaling Technology 9101S), rabbit anti-Cleaved Caspase-3 (1:1000; Cell Signaling Technology 9664), and mouse anti-GAPDH (1:10,000; Ambion Am4300).

### Fish

DNA probes for fluorescence in situ hybridization (FISH) were labeled by nick translation with Spectrum Green or Spectrum Red 2′-deoxyuridine-5′-triphospahte (Abbott). Cells were grown in 8-chamber slides and fixed with methanol: acetone = 1:1 fixation solution for 10 min at 4° C. BAC Fish clone, RP11-143123 was purchased from BACPAC company.

### Cell Migration Assay

The effect of AVIL on cell migration was assayed by a wound-healing assay according to previous publication (Qin, F., et al. Cancer Lett 2016 380:39-46). Briefly, cells were cultured to confluency. A wound was created by scraping the cells using a 10 ul plastic pipette tip, and the medium was replaced with fresh medium. Images were captured immediately after the scratch, and again 8h later. Cell migration was quantitively assessed by the size of the gap within the confluent monolayer culture at the end of the experiment. Eight gaps were measured. For siRNA experiment, the measurement took place around 48-72 h after transfection.

### RNA-Sequencing

RNA-Seq was performed by Axeq. Briefly, the mRNA in total RNA was converted into a library of template molecules suitable for subsequent cluster generation using the reagents provided in the Illumina TruSeq RNA Sample Preparation Kit. Paired-end sequencing was then conducted using Hiseq 2000 (Illumina). The 101-bp RNA-seq data were analyzed as before (Qin, F., et al. PLoS Genet 2015 11:e1005001). The circular image results of alignments were presented using Circos plot.

RNA sequencing data was preprocessed following FastQC output to check for quality issues. Alignment was done using Kallisto in paired-end mode, aligning to the cds GRCh38 build from Ensembl (Bray, N. L., et al. Nat Biotechnol 2016 34:525-527). Pseudoalignments were processed for differential analysis using tximport passing off to Deseq2 (Soneson, C., et al. F1000Res 2015 4:1521; Love, M. I., et al. Genome Biol 2014 15:550). After Deseq2 normalization and quantitation, volcano plots were generated with EnhancedVolcano, and pathway analysis was performed using both over-representation (ORA) and gene set enrichment analysis (GSEA) using clusterProfiler with the “DOSE” and nPerm = 10000 parameters for GSEA. Curated gene sets were taken from MsigDB with the associated reactomePA and Pathview packages (Yu, G., et al. Mol Biosyst 2016 12:477-479; Luo, W., et al. Bioinformatics 2013 29:1830-1831).

### Flow Cytometry

Trypsinized cells were spun down in 15ml conical tube and resuspend in 2.5ml PBS. 100% enthanol was added dropwise, and samples were put in -20° C. overnight. 3 uM solution of Propidium Iodide was prepared by diluting 1 mg/ml (1.5 mM) stock solution 1:500 in staining buffer (100 mM Tris, pH 7.4, 150 mM NaCl, 1 mM CaCl2,0.5 mM MgCl2, 0.1%NP-40). 1 mL was added to the cells, covered with dilute stain, for 15 minutes at room temperature and analyzed by flow cytometry in the presence of the dye.

### Live-Cell Imaging

The cellular movement was analyzed by live-cell imaging. Briefly, RH30, RD, SMS-CTR or MSC were plated to 30-40% confluency in DMEM + 10% FBS, followed by transfection with siGL2 or siAVIL. 2 h prior to start of imaging media was supplemented with 0.5 µM SiR-DNA (Cytoskeleton) dye. 24 h after siRNA transfection images were collected on a Zeiss Axio-observer-Z1 epifluorescent microscope in humidified chamber in 5% CO₂ environment, at 37° C. every 20 min over the period of 24 h. Resulting movies were processed using ImageJ, and cell movement was tracked semi-automatically based on SiR-DNA staining by TrackMate plugin for ImageJ.

### Tumor Formation in Vivo

The mouse work was performed under the study protocol approved by the University of Virginia Institutional Animal Care and Use Committee. Immunocompromised SCID/NCr BALB/c adult male mice (6-8 weeks old) were used. All animals were housed in sterilized plastic cages under specific pathogen-free conditions, at 22° C., 12/12 light/dark cycle, 55% humidity. RH30 or RD cells were transfected with control shRNA, or shAVIL. The transfected cells were then counted, and 3 × 10⁶ were injected subcutaneously. The tumors were harvested when most animals in the shCT group reached the human endpoint. The tumors were measured by caliber, and weighed. Since shAVIL group often had much smaller or no tumor, we waited longer before harvesting to show the survival differences. For the tet-inducible system, mice were fed with water containing doxycycline hydrochloride (2 mg/ml) and 5% sucrose. The doxycycline-sucrose solution was prepared fresh every three to four days, and kept in brown drinking bottle.

MSC stably expressing AVIL or MARS-AVIL or an empty vector were injected subcutaneously into the flanks of NIH-III Nude mice. On the same animal, the left side was injected with cells transfected with control vector, whereas the right with cells transfected with AVIL or MARS-AVIL expressing vector. Around two million cells were used per injection. The animals were monitored twice a week.

### Statistics and Reproducibility

All the experiments were repeated at least three times unless otherwise noted. All quantitative data were presented as the mean ± SEM (standard error of the mean) or the mean ± SD (standard deviation) as indicated of at least three independent experiments by Student’s t-test for between-group differences. Clinical prognosis was analyzed by Kaplan-Meier method and Log-rank test. The P< 0.05 was considered statistically significant.

### Data Access

RNA-Seq data for MSC cells overexpressing AVIL, and MSC control triplicates has been deposited into GEO database, under the accession: GSE180837.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for treating a cancer in a subject comprising contacting the at least one target cell with an effective amount of a silencing oligonucleotide that reduces the expression of the AVIL-encoding gene.

2. The method of claim 1, wherein the cancer is selected from brain cancer and cancerous tumors such as glioblastomas, rhabdosarcomas, gliomas, lung cancer, bladder cancer including bladder urothelial carcinoma, and renal cancer including kidney clear cell carcinoma.

3. The method of claim 1, wherein the cancer is a rhabdosarcoma.

4. The method of claim 1, wherein the silencing oligonucleotide is an siRNA.

5. The method of claim 4, wherein the siRNA comprises the nucleic acid sequence selected from the group consisting of SEQ ID NO:12 to 97 and 116 to 144.

6. The method of claim 1, wherein the silencing RNA is an shRNA.

7. The method of claim 6, wherein the shRNA comprises the nucleic acid sequence selected from the group consisting of SEQ ID NO:98 to 107.

8. The method of claim 1, wherein the silencing RNA is an sgRNA.

9. The method of claim 8, wherein the sgRNA comprises the nucleic acid sequence selected from the group consisting of SEQ ID NO:108 to 115.

10. The method of claim 1, further comprising at least one agent known to treat a cancer.

11. The method of claim 10, wherein the at least one agent known to treat a cancer is a hormone therapy agent; an alkylating agent, an antimetabolite agent, an antineoplastic antibiotic agent, a mitotic inhibitor agent, a mTor inhibitor agent, other chemotherapeutic agent, or combinations thereof.

12. The method of claim 10, wherein the at least one agent known to treat a cancer is a hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt thereof.

13. The method of claim 10, wherein the at least one agent known to treat a cancer is a antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

14. The method of claim 10, wherein the at least one agent known to treat a cancer is an antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

15. The method of claim 10, wherein the at least one agent known to treat a cancer is an alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt.

16. The method of claim 10, wherein the at least one agent known to treat a cancer is a mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt.

17. The method of claim 10, wherein the at least one agent known to treat a cancer is a mTor inhibitor agent is selected from one or more of the group consisting of everolimus, siroliumus, and temsirolimus, or a pharmaceutically acceptable salt thereof.

18. The method of claim 10, wherein the at least one agent known to treat a cancer is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α- ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin gefinitib, capecitabine, erlotinib, azacitidine, temozolomide, gemcitabine, vasostatin, and combinations thereof.
